(19) United States
(12) Patent Application Publication
(10) Pub. No.: US 2026/0347503 A1
(43) Pub. Date: Nov. 5, 2026

(54) METHODS OF TREATMENT AND PREVENTION OF POST-SURGICAL THROMBOSIS OR DISORDERS USING FACTOR XI (FXI) BINDING ANTIBODIES

(71) Applicant: Regeneron Pharmaceuticals, Inc., Tarrytown, NY (US)

(72) Inventors: Aaron Paul Kithcart, New York, NY (US); Meagan P. O'Brien, New York, NY (US); Oren Levy, New York, NY (US); Ethan Marin, Woodbridge, CT (US); Benjamin Adam Olenchock, Ridgefield, CT (US); David Ethan Gutstein, Short Hills, NJ (US); Andres Sirulnik, Irvington, NY (US); George D. Yancopoulos, Yorktown Heights, NY (US)

(21) Appl. No.: 19/422,964

(22) Filed: Dec. 17, 2025

Related U.S. Application Data
(60) Provisional application No. 63/734,901, filed on Dec. 17, 2024.
(60) Provisional application No. 63/735,599, filed on Dec. 18, 2024.
(60) Provisional application No. 63/737,297, filed on Dec. 20, 2024.
(60) Provisional application No. 63/799,042, filed on May 2, 2025.
(60) Provisional application No. 63/895,788, filed on Oct. 8, 2025.

(51) Int. Cl. C07K 16/36; A61K 31/4545; A61K 31/5377; A61K 31/616; A61K 31/727; A61K 39/00; A61P 7/02

(52) U.S. Cl. CPC C07K 16/36; A61K 31/4545; A61K 31/5377; A61K 31/616; A61K 31/727; A61P 7/02; A61K 2039/505; A61K 2039/54; A61K 2039/545; C07K 2317/565

(57) ABSTRACT

The present disclosure provides methods for using antibodies that bind to Factor XI (FXI) for the purpose of preventing or treating clot formation or thrombogenesis after a surgical procedure. According to certain embodiments, the antibodies are antagonist antibodies that inhibit blood clot formation via the intrinsic pathway without affecting hemostasis, as shown by their effect on prolonging aPTT without affecting PT. As such, these antagonist antibodies may be used to treat or prevent clot formation in a subject requiring such treatment, such as, but not limited to, post-surgery.

### RELATED APPLICATIONS

The instant application claims priority to U.S. Provisional Application No. 63/734,901, filed on Dec. 17, 2024; U.S. Provisional Application No. 63/735,599, filed on Dec. 18, 2024; U.S. Provisional Application No. 63/737,297, filed on Dec. 20, 2024; U.S. Provisional Application No. 63/799,042, filed May 2, 2025; and U.S. Provisional Application No. 63/895,788, filed on Oct. 8, 2025. The entire contents of each of the foregoing applications are expressly incorporated by reference herein.

### SEQUENCE LISTING

The instant application contains a Sequence Listing which has been submitted electronically in XML format and is hereby incorporated by reference in its entirety. Said XML copy, created on Dec. 15, 2025 is named 118003-02020.XML and is 203,683,579 bytes in size.

### FIELD

The present disclosure relates to methods of treatment, prevention, management, and/or reduction of the risk of post-surgical venous thromboembolism, and managing or reducing bleeding or bleeding risk in a subject using antibodies that bind to Factor XI (FXI) or a fragment thereof.

### BACKGROUND

Anticoagulants are medications used to treat and prevent venous and arterial thrombotic and embolic events by interfering with components of the coagulation cascade. The major complication of anticoagulants in clinical usage (such as warfarin, heparins, and direct oral coagulants) is bleeding, since these drugs not only reduce thrombus formation, but also impair hemostasis. Furthermore, the bleeding liability of current anticoagulants has been associated with their blockade of the extrinsic and common pathways of the coagulation system (Harter, 2015). A major goal in the development of novel anticoagulants is to minimize bleeding risk while maintaining efficacy; targeting the intrinsic pathway is hypothesized to preserve normal hemostatic mechanisms and, therefore, carry less bleeding risk.

Inhibition of Factor XI (FXI), a component of the intrinsic pathway, has emerged as a promising approach for effective anticoagulation (i.e., prevention of thrombus). Factor XI can be activated by FXIIa. Factor XIIa activation is the initial step in the intrinsic pathway of coagulation and can be triggered by contact with negatively charged molecules, including polyphosphates from bacteria or platelets, free nucleic acids, misfolded proteins, or nonbiological surfaces (Al-Horani, 2018). Selective inhibition of FXI activation by FXIIa represents a strategy to block thrombosis initiated by the intrinsic pathway triggers, while maintaining hemostatic mechanisms of the extrinsic pathway.

However, more studies are needed to determine the occurrence of thromboses and clotting disorders following surgical procedures using FXI inhibitors, while balancing the risk of bleeding.

### BRIEF SUMMARY

Provided herein are methods of use of exemplary isolated monoclonal antibodies, such as REGN7508 and REGN9933, and antigen-binding fragments thereof, that bind Factor XI (FXI) for treatment, prevention, management, and/or reduction of the risk of post-surgical venous thromboembolism (VTE) and/or complications associated therewith, and managing or reducing bleeding or bleeding risk in a subject. In some embodiments the subjects underwent surgery prior to any treatment with the antibodies. In some embodiments, the subject is at an increased risk of developing post-surgical venous thromboembolism (VTE), and/or complications associated therewith, as compared to a healthy individual.

REGN7508 is a monoclonal antibody which binds to FXI and prevents factor IX activation by FXIa, thereby mimicking FXI deficiency. Thus, REGN7508 may prevent pathologic thrombosis due to activation of the intrinsic pathway of coagulation, while potentially maintaining hemostatic mechanisms distal to FXIIa activation of FXI. A second antibody disclosed herein, REGN9933, is a monoclonal antibody which binds to FXI and prevents its activation by FXIIa. Thus, REGN9933 may prevent pathologic thrombosis due to activation of the intrinsic pathway of coagulation, while potentially maintaining hemostatic mechanisms of the extrinsic pathway.

In its broadest aspect, the disclosure provides post-surgical methods of use for anti-FXI antibodies and/or their antigen binding fragments, which block FXI activity or activation and reduce blood clot formation, while minimizing the risk of bleeding. These methods may be used to prevent, treat, reduce the incidence of, or reduce the negative effects of blood clot formation in the blood stream or tissue in a patient in need thereof after surgery. In some embodiments, the methods may be used to treat, prevent, manage, or reduce the occurrence of post-surgical venous thromboembolism and/or complications associated therewith, while managing or minimizing the risk of bleeding. Preferably, the methods attenuate thrombosis without perturbing hemostasis.

In certain embodiments, the methods may be useful to prevent, treat, or reduce the risk of venous blood clotting following surgery. In some embodiments, the surgery involves the use of anticoagulant therapy and where there is a risk to the patient of bleeding due to the use of anticoagulant therapy. In some embodiments, the methods may be useful to prevent, treat, or reduce the risk of post-surgical complications, e.g., venous thromboembolism (VTE).

In some embodiments, the subject is predisposed to develop post-surgical venous thromboembolism (VTE).

The antibodies for use in the disclosed methods can be full-length (for example, an IgG1 or IgG4 antibody) or may comprise only an antigen-binding portion (for example, a Fab, F(ab′)₂ or scFv fragment), and may be modified to affect functionality, e.g., to eliminate residual effector functions (Reddy et al., 2000, J. Immunol. 164:1925-1933).

Two exemplary anti-FXI antibodies for use in the present disclosure (REGN7508 and REGN9933) are listed in Table 1 herein. Table 1 sets forth the amino acid sequence identifiers and the nucleic acid sequence identifiers of exemplary heavy chain variable regions (HCVRs), light chain variable regions (LCVRs), heavy chain complementarity determining regions (HCDR1, HCDR2 and HCDR3), and light chain complementarity determining regions (LCDR1, LCDR2 and LCDR3) of the exemplary anti-FXI antibodies. Table 1 also sets forth the nucleic acid sequence identifiers of exemplary HCVRs, LCVRs, HCDR1, HCDR2 HCDR3, LCDR1, LCDR2 and LCDR3 of an exemplary anti-FXI antibody.

The present disclosure also provides antibodies or antigen-binding fragments thereof that bind FXI for use in the disclosed methods, comprising an HCVR comprising an amino acid sequence selected from any of the HCVR amino acid sequences listed in Table 1, or a substantially similar sequence thereof having at least about 90%, at least about 95%, at least about 98% or at least about 99% sequence identity thereto.

The present disclosure also provides antibodies or antigen-binding fragments thereof that bind FXI for use in the disclosed methods, comprising an LCVR comprising an amino acid sequence selected from any of the LCVR amino acid sequences listed in Table 1, or a substantially similar sequence thereof having at least about 90%, at least about 95%, at least about 98% or at least about 99% sequence identity thereto.

The present disclosure also provides antibodies or antigen-binding fragments thereof that bind FXI for use in the disclosed methods, comprising an HCVR and an LCVR amino acid sequence pair (HCVR/LCVR) comprising any of the HCVR amino acid sequences listed in Table 1 paired with any of the LCVR amino acid sequences listed in Table 1. According to certain embodiments, the present disclosure provides antibodies, or antigen-binding fragments thereof, for use in the disclosed methods, comprising an HCVR/LCVR amino acid sequence pair contained within any of the exemplary anti-FXI antibodies listed in Table 1.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In a first aspect, the disclosure provides a method of preventing, managing, or reducing the risk of a venous thromboembolism (VTE), or a complication or symptom associated with VTE following a surgical procedure in a subject, the method comprising: administering a single dose of an antibody, or antigen-binding portion thereof, that binds to Factor XI (FXI) to the subject, thereby preventing, managing, or reducing the risk of VTE, or the complication or symptom associated with VTE, following the surgical procedure in the subject. The disclosure also provides the use of an antibody, or antigen-binding portion thereof that binds to Factor XI (FXI), for the manufacture of a medicament for preventing, managing, or reducing the risk of a venous thromboembolism (VTE), or a complication or symptom associated with VTE following a surgical procedure in a subject.

In one embodiment, the administration does not substantially increase bleeding risk.

In one embodiment, the antibody, or antigen-binding portion thereof, comprises a heavy chain variable region (HCVR) comprising a heavy chain complementarity determining region (HCDR) 1, HCDR2, and HCDR3 within the HCVR, wherein the HCVR comprises the amino acid sequence of SEQ ID NO: 23; and a light chain variable region (LCVR) comprising a light chain complementarity determining region (LCDR) 1, LCDR2, and LCDR3 within the LCVR, wherein the LCVR comprises the amino acid sequence of SEQ ID NO: 31. In one embodiment, the antibody, or antigen-binding portion thereof, comprises a heavy chain variable region (HCVR) comprising a heavy chain complementarity determining region (HCDR) 1 sequence comprising SEQ ID NO: 25, a HCDR2 sequence comprising SEQ ID NO: 27, a HCDR3 sequence comprising SEQ ID NO: 29; and a light chain variable region (LCVR) comprising a light chain complementarity determining region (LCDR) 1 sequence comprising SEQ ID NO: 33, a LCDR2 sequence comprising SEQ ID NO: 35, and a LCDR3 sequence comprising SEQ ID NO: 37.

In one embodiment, the antibody, or antigen-binding portion thereof, is administered as an intravenous dose of about 5 mg to about 600 mg. In one embodiment, the antibody, or antigen-binding portion thereof, is administered as an intravenous dose of about 5 mg, about 15 mg, about 30 mg, about 60 mg, about 125 mg, about 250 mg, about 300 mg, about 350 mg, about 400 mg, about 450 mg, about 500 mg, about 550 mg, or about 600 mg. In one embodiment, the antibody, or antigen-binding portion thereof, is administered as an intravenous dose of about 250 mg. In one embodiment, the antibody, or antigen-binding portion thereof, is administered as an intravenous dose of about 300 mg. In any of the foregoing embodiments, the antibody may be administered as a single intravenous dose. In one embodiment, the antibody, or antigen-binding portion thereof, is administered intravenously as a single dose of about 250 mg within 3 hours of surgery. In one embodiment, the antibody, or antigen-binding fragment thereof, is administered 3 hours after surgery.

In one embodiment, the antibody, or antigen-binding portion thereof, is administered as a subcutaneous dose of about 100 mg to about 600 mg. In one embodiment, the antibody, or antigen-binding portion thereof, is administered as a subcutaneous dose of about 300 mg to about 600 mg. In one embodiment, the antibody, or antigen-binding portion thereof, is administered as a subcutaneous dose of about 125 mg, about 150 mg, about 200 mg, about 250 mg, about 300 mg, about 350 mg, about 400 mg, about 450 mg, about 500 mg, about 550 mg, or about 600 mg. In one embodiment, the antibody, or antigen-binding portion thereof, is administered as a subcutaneous dose of about 250 mg. In one embodiment, the antibody, or antigen-binding portion thereof, is administered as a subcutaneous dose of about 300 mg. In one embodiment, the antibody, or antigen-binding portion thereof, is administered as a subcutaneous dose of about 600 mg. In any of the foregoing embodiments, the antibody may be administered as a single subcutaneous dose. In another embodiment, the antibody, or antigen-binding portion thereof, is administered subcutaneously at a dose of about 300 mg within 2 to 4 hours of surgery. In one embodiment, the antibody, or antigen-binding fragment thereof, is administered 2 to 4 hours after surgery.

In one embodiment, the antibody, or antigen-binding portion thereof, is administered as an intravenous dose of about 3 mg to about 600 mg. In one embodiment, the antibody, or antigen-binding portion thereof, is administered as an intravenous dose of about 3 mg, about 10 mg, about 11 mg, about 30 mg, about 100 mg, about 150 mg, about 200 mg, about 250 mg, about 300 mg, about 250 mg, about 400 mg, about 450 mg, about 500 mg, about 550 mg, or about 600 mg. In one embodiment, the antibody, or antigen-binding portion thereof, is administered as an intravenous dose of about 3 mg. In one embodiment, the antibody, or antigen-binding portion thereof, is administered as an intravenous dose of about 10 mg. In one embodiment, the antibody, or antigen-binding portion thereof, is administered as an intravenous dose of about 30 mg. In one embodiment, the antibody, or antigen-binding portion thereof, is administered as an intravenous dose of about 100 mg. In one embodiment, the antibody, or antigen-binding portion thereof, is administered as an intravenous dose of about 150 mg. In one embodiment, the antibody, or antigen-binding portion thereof, is administered as an intravenous dose of about 200 mg. In one embodiment, the antibody, or antigen-binding portion thereof, is administered as an intravenous dose of about 250 mg. In one embodiment, the antibody, or antigen-binding portion thereof, is administered as an intravenous dose of about 300 mg. In one embodiment, the antibody, or antigen-binding portion thereof, is administered as an intravenous dose of about 350 mg. In one embodiment, the antibody, or antigen-binding portion thereof, is administered as an intravenous dose of about 400 mg. In one embodiment, the antibody, or antigen-binding portion thereof, is administered as an intravenous dose of about 450 mg. In one embodiment, the antibody, or antigen-binding portion thereof, is administered as an intravenous dose of about 500 mg. In one embodiment, the antibody, or antigen-binding portion thereof, is administered as an intravenous dose of about 550 mg. In one embodiment, the antibody, or antigen-binding portion thereof, is administered as an intravenous dose of about 600 mg. In any of the foregoing embodiments, the antibody may be administered as a single intravenous dose. In one embodiment, the antibody, or antigen-binding portion thereof, is administered as a subcutaneous dose of about 100 mg to about 600 mg, about 100 mg to about 1000 mg, or about 300 mg to about 1000 mg. In one embodiment, the antibody, or antigen-binding portion thereof, is administered as a subcutaneous dose of about 100 mg, about 200 mg, about 250 mg, about 300 mg, about 400 mg, about 500 mg, about 600 mg, about 700 mg, about 800 mg, about 900 mg, or about 1000 mg. In one embodiment, the antibody, or antigen-binding portion thereof, is administered as a subcutaneous dose of about 100 mg. In one embodiment, the antibody, or antigen-binding portion thereof, is administered as a subcutaneous dose of about 200 mg. In one embodiment, the antibody, or antigen-binding portion thereof, is administered as a subcutaneous dose of about 250 mg. In one embodiment, the antibody, or antigen-binding portion thereof, is administered as a subcutaneous dose of about 300 mg. In one embodiment, the antibody, or antigen-binding portion thereof, is administered as a subcutaneous dose of about 350 mg. In one embodiment, the antibody, or antigen-binding portion thereof, is administered as a subcutaneous dose of about 400 mg. In one embodiment, the antibody, or antigen-binding portion thereof, is administered as a subcutaneous dose of about 450 mg. In one embodiment, the antibody, or antigen-binding portion thereof, is administered as a subcutaneous dose of about 500 mg. In one embodiment, the antibody, or antigen-binding portion thereof, is administered as a subcutaneous dose of about 550 mg. In one embodiment, the antibody, or antigen-binding portion thereof, is administered as a subcutaneous dose of about 600 mg. In one embodiment, the antibody, or antigen-binding portion thereof, is administered as a subcutaneous dose of about 700 mg. In one embodiment, the antibody, or antigen-binding portion thereof, is administered as a subcutaneous dose of about 800 mg. In one embodiment, the antibody, or antigen-binding portion thereof, is administered as a subcutaneous dose of about 900 mg. In one embodiment, the antibody, or antigen-binding portion thereof, is administered as a subcutaneous dose of about 1000 mg. In any of the foregoing embodiments, the antibody may be administered as a single subcutaneous dose.

In one embodiment, the antibody, or antigen-binding portion thereof, is for administration at a dose of about 200 mg to about 600 mg. In one embodiment, the antibody, or antigen-binding portion thereof, is administered at a dose of about 200 mg to about 600 mg. In one embodiment, the antibody, or antigen-binding portion thereof, is for administration at a single intravenous dose of about 250 mg. In one embodiment, the antibody, or antigen-binding portion thereof, is for administration at a single subcutaneous dose of about 30 mg.

In one embodiment, the administering occurs before surgery or the surgical procedure. In one embodiment, the administering occurs about 6 hours, about 12 hours, about 18 hours, about 24 hours before surgery. In one embodiment, the administering occurs about 6 hours to about 24 hours before surgery. In another embodiment, the administering occurs about 12 to about 24 hours before surgery. In another embodiment, the administering occurs about 6 to about 18 hours before surgery. In another embodiment, the administering occurs about 1 hours, 2 hours, 3 hours, 4 hours, or 5 hours before surgery. In one embodiment, the administering occurs during surgery.

In one embodiment, the administering occurs after surgery. In one embodiment, the administering occurs within 36 hours after the surgical procedure. In one embodiment, the administering occurs between 24 hours and 36 hours after the surgical procedure. In one embodiment, the administering occurs at about 2 hours, 3 hours, 4 hours, 5 hours, 6 hours, 7 hours, 8 hours, 9 hours, 10 hours, 12 hours, 16 hours, 20 hours, 24 hours, 28 hours, 32 hours, or 36 hours after the surgical procedure. In one embodiment, the administering occurs within 24 hours after the surgical procedure. In one embodiment, the administering occurs between 1 hour and 12 hours, between 6 hours and 18 hours, or between 12 hours and 24 hours after the surgical procedure. In one embodiment, the administering occurs between about 1 hour and about 6 hours after the surgical procedure. In another embodiment, the administering occurs between about 2 hours and about 4 hours after the surgical procedure. In another embodiment, the administering occurs within 3 hours of the surgical procedure. In one embodiment, the administering occurs 3 hours after the surgical procedure. In one embodiment, the administering occurs at about 1 hour, about 2 hours, about 3 hours, about 4 hours, about 8 hours, about 12 hours, about 16 hours, about 20 hours, about 24 hours after the surgical procedure.

In one embodiment, the antibody, or antigen-binding portion thereof, comprises a HCVR sequence comprising at least 90% identity to SEQ ID NO: 23 and a LCVR sequence comprising at least 90% identity to SEQ ID NO: 31. In one embodiment, the antibody, or antigen-binding portion thereof, comprises a HCVR sequence comprising SEQ ID NO: 23 and a LCVR sequence comprising SEQ ID NO: 31. In one embodiment, the antibody, or antigen-binding portion thereof, comprises a heavy chain (HC) sequence comprising SEQ ID NO: 21 and a light chain (LC) sequence comprising SEQ ID NO: 39.

In one embodiment, the antibody, or antigen-binding portion thereof, comprises an HCDR1 sequence comprising SEQ ID NO: 25, an HCDR2 sequence comprising SEQ ID NO: 27, and an HCDR3 sequence comprising SEQ ID NO: 29. In one embodiment, the antibody, or antigen-binding portion thereof, comprises a LCDR1 sequence comprising SEQ ID NO: 33, a LCDR2 sequence comprising SEQ ID NO: 35, and a LCDR3 sequence comprising SEQ ID NO: 37. In one embodiment, the antibody, or antigen-binding portion thereof, comprises a HCVR sequence comprising SEQ ID NO: 3 and a LCVR sequence comprising SEQ ID NO: 13. In one embodiment, the antibody, or antigen-binding portion thereof, comprises a heavy chain (HC) sequence comprising SEQ ID NO: 1 and a light chain (LC) sequence comprising SEQ ID NO: 11.

In one embodiment, the method further comprises administering a single dose of a second antibody, or antigen-binding portion thereof, that binds to Factor XI (FXI) to the subject wherein the second antibody, or antigen-binding portion thereof, comprises a heavy chain variable region (HCVR) comprising a heavy chain complementarity determining region (HCDR) 1 sequence comprising SEQ ID NO: 5, a HCDR2 sequence comprising SEQ ID NO: 7, a HCDR3 sequence comprising SEQ ID NO: 9; and a light chain variable region (LCVR) comprising a light chain complementarity determining region (LCDR) 1 sequence comprising SEQ ID NO: 15, a LCDR2 sequence comprising SEQ ID NO: 17, and a LCDR3 sequence comprising SEQ ID NO: 19.

In one embodiment, the administering occurs before surgery or the surgical procedure. In one embodiment, the administering occurs about 1 hour, about 2 hours, about 3 hours, about 4 hours, about 5 hours, about 6 hours, about 12 hours, about 18 hours, about 24 hours before surgery. In one embodiment, the administering occurs about 6 hours to about 24 hours before surgery. In another embodiment, the administering occurs about 12 to about 24 hours before surgery. In another embodiment, the administering occurs about 6 to about 18 hours before surgery. In another embodiment, the administering occurs about 1 hours, 2 hours, 3 hours, 4 hours, or 5 hours before surgery. In one embodiment, the administering occurs during surgery. In one embodiment, the administering occurs after surgery. In one embodiment, the administering occurs within 36 hours of the surgical procedure. In one embodiment, the administering occurs within 24 hours after the surgical procedure. In one embodiment, the administering occurs between 12 hours and 24 hours after the surgical procedure. In one embodiment, the administering occurs between about 1 hour and about 6 hours after the surgical procedure. In another embodiment, the administering occurs between about 2 hours and about 4 hours after the surgical procedure. In another embodiment, the administering occurs within about 3 hours of the surgical procedure.

In a second aspect, the present disclosure provides a method of preventing, managing, or reducing the risk of a venous thromboembolism (VTE), or a complication or symptom associated with VTE, following a surgical procedure in a subject, the method comprising: administering a single dose of an antibody, or antigen-binding portion thereof, that binds to FXI to the subject, wherein the antibody, or antigen-binding portion thereof, comprises a heavy chain variable region (HCVR) comprising a heavy chain complementarity determining region (HCDR) 1 sequence comprising SEQ ID NO: 5, a HCDR2 sequence comprising SEQ ID NO: 7, a HCDR3 sequence comprising SEQ ID NO: 9; and a light chain variable region (LCVR) comprising a light chain complementarity determining region (LCDR) 1 sequence comprising SEQ ID NO: 15, a LCDR2 sequence comprising SEQ ID NO: 17, and a LCDR3 sequence comprising SEQ ID NO: 19; thereby preventing, managing, or reducing the risk of the VTE, or the complication or symptom associated with VTE, following the surgical procedure in the subject.

In one embodiment, the antibody, or antigen-binding portion thereof, is administered as an intravenous dose of about 3 mg to about 600 mg. In one embodiment, the antibody, or antigen-binding portion thereof, is administered as an intravenous dose of about 3 mg, about 10 mg, about 11 mg, about 30 mg, about 100 mg, about 150 mg, about 200 mg, about 250 mg, about 300 mg, about 250 mg, about 400 mg, about 450 mg, about 500 mg, about 550 mg, or about 600 mg. In one embodiment, the antibody, or antigen-binding portion thereof, is administered as an intravenous dose of about 3 mg. In one embodiment, the antibody, or antigen-binding portion thereof, is administered as an intravenous dose of about 10 mg. In one embodiment, the antibody, or antigen-binding portion thereof, is administered as an intravenous dose of about 30 mg. In one embodiment, the antibody, or antigen-binding portion thereof, is administered as an intravenous dose of about 100 mg. In one embodiment, the antibody, or antigen-binding portion thereof, is administered as an intravenous dose of about 150 mg. In one embodiment, the antibody, or antigen-binding portion thereof, is administered as an intravenous dose of about 200 mg. In one embodiment, the antibody, or antigen-binding portion thereof, is administered as an intravenous dose of about 250 mg. In one embodiment, the antibody, or antigen-binding portion thereof, is administered as an intravenous dose of about 300 mg. In one embodiment, the antibody, or antigen-binding portion thereof, is administered as an intravenous dose of about 350 mg. In one embodiment, the antibody, or antigen-binding portion thereof, is administered as an intravenous dose of about 400 mg. In one embodiment, the antibody, or antigen-binding portion thereof, is administered as an intravenous dose of about 450 mg. In one embodiment, the antibody, or antigen-binding portion thereof, is administered as an intravenous dose of about 500 mg. In one embodiment, the antibody, or antigen-binding portion thereof, is administered as an intravenous dose of about 550 mg. In one embodiment, the antibody, or antigen-binding portion thereof, is administered as an intravenous dose of about 600 mg. In any of the foregoing embodiments, the antibody may be administered as a single intravenous dose. In one embodiment, the antibody, or antigen-binding portion thereof, is administered as a subcutaneous dose of about 100 mg to about 600 mg, about 100 mg to about 1000 mg, or about 300 mg to about 1000 mg. In one embodiment, the antibody, or antigen-binding portion thereof, is administered as a subcutaneous dose of about 100 mg, about 200 mg, about 250 mg, about 300 mg, about 400 mg, about 500 mg, about 600 mg, about 700 mg, about 800 mg, about 900 mg, or about 1000 mg. In one embodiment, the antibody, or antigen-binding portion thereof, is administered as a subcutaneous dose of about 100 mg. In one embodiment, the antibody, or antigen-binding portion thereof, is administered as a subcutaneous dose of about 200 mg. In one embodiment, the antibody, or antigen-binding portion thereof, is administered as a subcutaneous dose of about 250 mg. In one embodiment, the antibody, or antigen-binding portion thereof, is administered as a subcutaneous dose of about 300 mg. In one embodiment, the antibody, or antigen-binding portion thereof, is administered as a subcutaneous dose of about 350 mg. In one embodiment, the antibody, or antigen-binding portion thereof, is administered as a subcutaneous dose of about 400 mg. In one embodiment, the antibody, or antigen-binding portion thereof, is administered as a subcutaneous dose of about 450 mg. In one embodiment, the antibody, or antigen-binding portion thereof, is administered as a subcutaneous dose of about 500 mg. In one embodiment, the antibody, or antigen-binding portion thereof, is administered as a subcutaneous dose of about 550 mg. In one embodiment, the antibody, or antigen-binding portion thereof, is administered as a subcutaneous dose of about 600 mg. In one embodiment, the antibody, or antigen-binding portion thereof, is administered as a subcutaneous dose of about 700 mg. In one embodiment, the antibody, or antigen-binding portion thereof, is administered as a subcutaneous dose of about 800 mg. In one embodiment, the antibody, or antigen-binding portion thereof, is administered as a subcutaneous dose of about 900 mg. In one embodiment, the antibody, or antigen-binding portion thereof, is administered as a subcutaneous dose of about 1000 mg. In any of the foregoing embodiments, the antibody may be administered as a single subcutaneous dose.

In one embodiment, the administering occurs before surgery or the surgical procedure. In one embodiment, the administering occurs about 6 hours, about 12 hours, about 18 hours, about 24 hours before surgery. In one embodiment, the administering occurs about 6 hours to about 24 hours before surgery. In another embodiment, the administering occurs about 12 to about 24 hours before surgery. In another embodiment, the administering occurs about 6 to about 18 hours before surgery. In another embodiment, the administering occurs about 1 hours, 2 hours, 3 hours, 4 hours, or 5 hours before surgery. In one embodiment, the administering occurs during surgery. In one embodiment, the administering occurs after surgery. In one embodiment, the administering occurs within 36 hours after the surgical procedure. In one embodiment, the administering occurs between 24 hours and 36 hours after the surgical procedure. In one embodiment, the administering occurs at about 28 hours, about 32 hours, or about 36 hours after the surgical procedure. In one embodiment, the administering occurs within 24 hours after the surgical procedure. In one embodiment, the administering occurs between 1 hour and 12 hours, between 6 hours and 18 hours, or between 12 hours and 24 hours after the surgical procedure. In one embodiment, the administering occurs at about 1 hour, about 4 hours, about 8 hours, about 12 hours, about 16 hours, about 20 hours, about 24 hours after the surgical procedure.

In one embodiment, the antibody, or antigen-binding portion thereof, comprises a HCVR sequence comprising at least 90% identity to SEQ ID NO: 3 and a LCVR sequence comprising at least 90% identity to SEQ ID NO: 13. In one embodiment, the antibody, or antigen-binding portion thereof, comprises a HCVR sequence comprising SEQ ID NO: 3 and a LCVR sequence comprising SEQ ID NO: 13. In one embodiment, the antibody, or antigen-binding portion thereof, comprises a heavy chain (HC) sequence comprising SEQ ID NO: 1 and a light chain (LC) sequence comprising SEQ ID NO: 11.

In one embodiment, the method further comprises administering a single dose of a second antibody, or antigen-binding portion thereof, that binds to Factor XI (FXI) to the subject within 36 hours of the surgical procedure, wherein the second antibody, or antigen-binding portion thereof, comprises a heavy chain variable region (HCVR) comprising a heavy chain complementarity determining region (HCDR) 1 sequence comprising SEQ ID NO: 25, a HCDR2 sequence comprising SEQ ID NO: 27, a HCDR3 sequence comprising SEQ ID NO: 29; and a light chain variable region (LCVR) comprising a light chain complementarity determining region (LCDR) 1 sequence comprising SEQ ID NO: 33, a LCDR2 sequence comprising SEQ ID NO: 35, and an LCDR3 sequence comprising SEQ ID NO: 37.

In one embodiment, the administering occurs before surgery or the surgical procedure. In one embodiment, the administering occurs about 6 hours, about 12 hours, about 18 hours, about 24 hours before surgery. In one embodiment, the administering occurs about 6 hours to about 24 hours before surgery. In another embodiment, the administering occurs about 12 to about 24 hours before surgery. In another embodiment, the administering occurs about 6 to about 18 hours before surgery. In another embodiment, the administering occurs about 1 hours, 2 hours, 3 hours, 4 hours, or 5 hours before surgery. In one embodiment, the administering occurs during surgery. In one embodiment, the administering occurs after surgery. In one embodiment, the administering occurs within 36 hours of the surgical procedure. In one embodiment, the administering occurs within 24 hours after the surgical procedure. In one embodiment, the administering occurs between 12 hours and 24 hours after the surgical procedure.

In one embodiment, the method further comprises administering one or more anti-coagulants in combination with the antibody, or antigen-binding portion thereof. In one embodiment, the one or more anti-coagulants is selected from the group consisting of aspirin, enoxaparin, apixaban and rivaroxaban. In one embodiment, the anti-coagulant is aspirin. In one embodiment, the method further comprises administering aspirin in combination with the antibody, or antigen-binding portion thereof. In one embodiment, the aspirin is administered at a dosage of about 50 mg to about 100 mg per day for at least 5 days following the surgical procedure. In one embodiment, the aspirin is administered at a dosage of about 75 mg per day to the subject for at least 12 days following the surgical procedure. In one embodiment, the aspirin is administered orally at a dosage of about 75 mg daily for about 7 to about 14 days after surgery. In one embodiment, the anti-coagulant is enoxaparin. In one embodiment, the enoxaparin is subcutaneously administered at a dosage of about 40 mg daily for about 7 to about 14 days after surgery. In one embodiment, the anti-coagulant is apixaban. In one embodiment, the apixaban is orally administered once or twice daily at a dosage of about 2.5 mg for about 7 to about 14 days after surgery. In one embodiment, the anti-coagulant is rivaroxaban. In one embodiment, the rivaroxaban is orally administered at a dosage of about 15 mg or about 20 mg daily for about 7 to about 14 days after surgery.

In one embodiment, the surgical procedure is a cardiac, vascular, thoracic, orthopedic, trauma, abdominopelvic, gynecologic, urologic, bariatric, plastic, reconstructive, otolaryngic, arthroscopic, transplant, or neurologic surgery. In one embodiment, the surgical procedure is selected from the group consisting of total knee arthroplasty (TKA), hip arthroplasty, spinal cord injury surgery, and microvascular decompression surgery. In one embodiment, the transplant surgical procedure is selected from the consisting of heart, liver, lung, and kidney transplant surgery. In one embodiment, the complication or symptom associated with VTE is lower extremity swelling, warmth, redness, pain, shortness of breath, chest pain exacerbated by inspiration, hemoptysis, bleeding at the surgical site or elsewhere, or a combination thereof.

In one embodiment, the risk of thrombosis in the subject is decreased by at least 10%, at least 25%, at least 35%, at least 50%, at least 75%, at least 80%, at least 85%, at least 90%, or at least 95% following the administering step. In one embodiment, the risk of thrombosis in the subject following the administering step is decreased as compared to a control, wherein the control is an average measurement of thrombosis gathered from a population of subjects undergoing the surgical procedure who have not been administered the antibody, or antigen-binding portion thereof.

In one embodiment, the risk of bleeding in the subject following the administration step is not increased. In one embodiment, the risk of bleeding in the subject following the administration step is not increased as compared to a control, wherein the control is an average measurement of bleeding gathered from a population of subjects undergoing the surgical procedure who have not been administered the antibody, or antigen-binding portion thereof. In either embodiment, the risk of bleeding is not significantly increased following the administration step. In one embodiment, the risk of bleeding is decreased following the administration step.

In one embodiment, the administering results in prolonging activated partial thromboplastin time (aPTT), or reducing thrombin activity in plasma of the subject. In one embodiment, the antibody, or antigen-binding fragment thereof, increases activated partial thromboplastin time (aPTT) by at least two-fold, by at least 2.5-fold, by at least 3-fold, by at least 3.5-fold, or by at least 3.8-fold. In one embodiment, the administering results in prolonging activated partial thromboplastin time (aPTT), or reducing thrombin activity in plasma of the subject. In one embodiment, the antibody, or antigen-binding fragment thereof, increases activated partial thromboplastin time (aPTT) by about 2-fold to about 4-fold. In one embodiment, the antibody, or antigen-binding fragment thereof, does not increase prothrombin time (PT). In one embodiment, the antibody, or antigen-binding fragment thereof, inhibits FXIa-mediated thrombin activity by at least 5%, by at least 10%, by at least 15%, or by 5%-15%.

In one embodiment, the subject is a human subject. In one embodiment, the method further comprises a step of selecting the subject prior to the administration step. In one embodiment, the subject is selected on the basis of at least one of the following criteria: (a) is 50 years of age or greater at the time of the surgical procedure; (b) has a body weight of less than or equal to 130 kg at the time of the surgical procedure; (c) has a platelet count of <150K at the time of the surgical procedure; (d) has a normal international normalized ratio (INR) at the time of the surgical procedure; (e) has a normal aPTT at the time of the surgical procedure; (f) has hemoglobin>10/dL at the time of the surgical procedure; (g) has an alanine transaminase (ALT) level of <3× upper limit of normal (ULN) at the time of the surgical procedure; (h) has an aspartate transaminase (AST) level of <3× ULN at the time of the surgical procedure; and/or (i) has a total bilirubin level of <2× ULN at the time of the surgical procedure. In one embodiment, the subject is not selected if the subject exhibits at least one of the following criteria: (a) bleeding requiring hospitalization or transfusion in the six months prior to the surgical procedure; (b) intracranial or intraocular bleeding; excessive operative or post-operative bleeding; traumatic spinal or epidural anesthesia; bleeding diathesis such as Hemophilia A, Hemophilia B, or von Willebrand's Factor Deficiency; (c) thromboembolic disease or thrombophilia; (d) brain, spinal, or ocular surgery within the six months prior to the surgical procedure; (e) trauma within six months prior to the surgical procedure; (f) a hospitalization for greater than 24 hours for any reason within 30 days of the surgical procedure; (g) an estimated glomerular filtration rate (eGFR) of <45 mL/min/1.73 m² at the time of the surgical procedure; (h) a history of hypersensitivity or contraindication to the antibody, or antigen-binding portion thereof, (i) a contraindication to anticoagulation; (j) has received preoperative enoxaparin within 24 hours prior to the surgical procedure; (k) has an allergy to contrast agents; (1) has poor venous access; (m) expected post-surgical use of an epidural or a spinal catheter; (n) has had excessive intra-operative blood loss during the surgical procedure; (o) has a history of alcohol or drug abuse; (p) has a history of HIV infection or ongoing chronic hepatitis B or C infection; (q) has any malignancy, excluding nonmelanoma skin cancer or cervical/anus in situ that have been resected with no evidence of metastatic disease for three years prior to the surgical procedure; (r) has a history of significant multiple or severe allergies, or has had an anaphylactic reaction to prescription or nonprescription drugs and/or food; (s) has received a COVID-19 vaccination within one week of the surgical procedure; and/or (t) is a female subject of childbearing potential.

In one embodiment, the antibody, or antigen-binding fragment thereof, inhibits Factor XI functional activity (FXI:C) by at least 85%, or by at least 90%. In one embodiment, the inhibition is maintained for at least 20, at least 30, or at least 40 days post-dose.

In one embodiment, the antibody, or antigen binding portion thereof, is administered intravenously at a dose of about 30 mg to achieve a maximum serum concentration (Cₘₐₓ) of about 2.0 mg/L to about 2.5 mg/L. In one embodiment, the antibody, or antigen binding portion thereof, is administered intravenously at a dose of about 30 mg to achieve a dose-normalized serum concentration (C_{max/dose}) of about 0.05 mg/L/mg to 0.15 mg/L/mg. In one embodiment, the antibody, or antigen binding portion thereof, is administered intravenously at a dose of about 30 mg to achieve a time to Cₘₐₓ (Tₘₐₓ) of about 1.0 day to about 2.0 days. In one embodiment, the antibody, or antigen binding portion thereof, is administered intravenously at a dose of about 30 mg to achieve a time to last measurable serum concentration (Tₗₐₛₜ) of about 1.0 day to about 2.0 days. In one embodiment, the antibody, or antigen binding portion thereof, is administered intravenously at a dose of about 30 mg to achieve an area under the plasma concentration-time curve to last measurable serum concentration (AUCₗₐₛₜ) of about 1.5 day*mg/L to about 2.5 day*mg/L. In one embodiment, the antibody, or antigen binding portion thereof, is administered intravenously at a dose of about 30 mg to achieve a dose-normalized area under the plasma concentration-time curve from administration to last measurable serum concentration (AUC_{last/dose}) of about 0.05 day*mg/L/mg to about 0.10 day*mg/L/mg.

In one embodiment, the antibody, or antigen binding portion thereof, is administered intravenously at a dose of about 60 mg to achieve a maximum serum concentration (Cₘₐₓ) of about 7.0 mg/L to about 8.0 mg/L. In one embodiment, the antibody, or antigen binding portion thereof, is administered intravenously at a dose of about 60 mg to achieve a dose-normalized serum concentration (C_{max/dose}) of about 0.10 mg/L/mg to 0.2 mg/L/mg. In one embodiment, the antibody, or antigen binding portion thereof, is administered intravenously at a dose of about 60 mg to achieve a time to Cₘₐₓ (Tₘₐₓ) of about 0.5 days to about 1.5 days. In one embodiment, the antibody, or antigen binding portion thereof, is administered intravenously at a dose of about 60 mg to achieve a time to last measurable serum concentration (Tₗₐₛₜ) of about 5.0 days to about 6.0 days. In one embodiment, the antibody, or antigen binding portion thereof, is administered intravenously at a dose of about 60 mg to achieve an area under the plasma concentration-time curve to last measurable serum concentration (AUCₗₐₛₜ) of about 18 day*mg/L to about 20 day*mg/L. In one embodiment, the antibody, or antigen binding portion thereof, is administered intravenously at a dose of about 60 mg to achieve a dose-normalized area under the plasma concentration-time curve from administration to last measurable serum concentration (AUC_{last/dose}) of about 0.30 day*mg/L/mg to about 0.40 day*mg/L/mg.

In one embodiment, the antibody, or antigen binding portion thereof, is administered intravenously at a dose of about 125 mg to achieve a rate of clearance (CL) of about 1.0 L/day to about 1.5 L/day. In one embodiment, the antibody, or antigen binding portion thereof, is administered intravenously at a dose of about 125 mg to achieve a maximum serum concentration (Cₘₐₓ) of about 17.0 mg/L to about 19.0 mg/L. In one embodiment, the antibody, or antigen binding portion thereof, is administered intravenously at a dose of about 125 mg to achieve a dose-normalized serum concentration (C_{max/dose}) of about 0.10 mg/L/mg to 0.2 mg/L/mg. In one embodiment, the antibody, or antigen binding portion thereof, is administered intravenously at a dose of about 125 mg to achieve a steady-state volume of distribution (Vₛₛ) of about 5.0 L to about 7.5 L. In one embodiment, the antibody, or antigen binding portion thereof, is administered intravenously at a dose of about 125 mg to achieve a time to Cₘₐₓ (Tₘₐₓ) of about 0.5 days to about 1.5 days. In one embodiment, the antibody, or antigen binding portion thereof, is administered intravenously at a dose of about 125 mg to achieve a time to last measurable serum concentration (Tₗₐₛₜ) of about 11 days to about 14 days. In one embodiment, the antibody, or antigen binding portion thereof, is administered intravenously at a dose of about 125 mg to achieve a serum concentration half-life (T_{1/2}) of about 2.5 days to about 3.5 days. In one embodiment, the antibody, or antigen binding portion thereof, is administered intravenously at a dose of about 125 mg to achieve an area under the plasma concentration-time curve to last measurable serum concentration (AUCₗₐₛₜ) of about 85 day*mg/L to about 95 day*mg/L. In one embodiment, the antibody, or antigen binding portion thereof, is administered intravenously at a dose of about 125 mg to achieve a dose-normalized area under the plasma concentration-time curve from administration to last measurable serum concentration (AUC_{last/dose}) of about 0.65 day*mg/L/mg to about 0.75 day*mg/L/mg. In one embodiment, the antibody, or antigen binding portion thereof, is administered intravenously at a dose of about 125 mg to achieve an area under the plasma concentration-time curve extrapolated from administration to infinity (AUC_{inf}) of about 120 day*mg/L to about 130 day*mg/L. In one embodiment, the antibody, or antigen binding portion thereof, is administered intravenously at a dose of about 125 mg to achieve a dose-normalized area under the plasma concentration-time curve extrapolated from administration to infinity (AUC_{inf/dose}) of about 0.5 day*mg/L/mg to about 1.0 day*mg/L/mg. In one embodiment, the antibody, or antigen binding portion thereof, is administered intravenously at a dose of about 125 mg to achieve an area under the plasma concentration-time curve extrapolated from administration to infinity as a percentage of the total area under the plasma concentration-time curve (AUCₑₓₜᵣₐₚ) of about 4.0% to about 5.0%.

In one embodiment, the antibody, or antigen binding portion thereof, is administered intravenously at a dose of about 250 mg to achieve a rate of clearance (CL) of about 0.5 L/day to about 1.0 L/day. In one embodiment, the antibody, or antigen binding portion thereof, is administered intravenously at a dose of about 250 mg to achieve a maximum serum concentration (Cₘₐₓ) of about 42.0 mg/L to about 47.0 mg/L. In one embodiment, the antibody, or antigen binding portion thereof, is administered intravenously at a dose of about 250 mg to achieve a dose-normalized serum concentration (C_{max/dose}) of about 0.10 mg/L/mg to 0.2 mg/L/mg. In one embodiment, the antibody, or antigen binding portion thereof, is administered intravenously at a dose of about 250 mg to achieve a steady-state volume of distribution (Vₛₛ) of about 5.0 L to about 7.5 L. In one embodiment, the antibody, or antigen binding portion thereof, is administered intravenously at a dose of about 250 mg to achieve a time to Cₘₐₓ (Tₘₐₓ) of about 0.5 days to about 1.5 days. In one embodiment, the antibody, or antigen binding portion thereof, is administered intravenously at a dose of about 250 mg to achieve a time to last measurable serum concentration (Tₗₐₛₜ) of about 27 days to about 32 days. In one embodiment, the antibody, or antigen binding portion thereof, is administered intravenously at a dose of about 250 mg to achieve a serum concentration half-life (T_{1/2}) of about 4.5 days to about 5.5 days. In one embodiment, the antibody, or antigen binding portion thereof, is administered intravenously at a dose of about 250 mg to achieve an area under the plasma concentration-time curve to last measurable serum concentration (AUCₗₐₛₜ) of about 370 day*mg/L to about 380 day*mg/L. In one embodiment, the antibody, or antigen binding portion thereof, is administered intravenously at a dose of about 250 mg to achieve a dose-normalized area under the plasma concentration-time curve from administration to last measurable serum concentration (AUC_{last/dose}) of about 1.4 day*mg/L/mg to about 1.6 day*mg/L/mg. In one embodiment, the antibody, or antigen binding portion thereof, is administered intravenously at a dose of about 250 mg to achieve an area under the plasma concentration-time curve extrapolated from administration to infinity (AUC_{inf}) of about 385 day*mg/L to about 395 day*mg/L. In one embodiment, the antibody, or antigen binding portion thereof, is administered intravenously at a dose of about 250 mg to achieve a dose-normalized area under the plasma concentration-time curve extrapolated from administration to infinity (AUC_{inf/dose}) of about 1.3 day*mg/L/mg to about 1.7 day*mg/L/mg. In one embodiment, the antibody, or antigen binding portion thereof, is administered intravenously at a dose of about 250 mg to achieve an area under the plasma concentration-time curve extrapolated from administration to infinity as a percentage of the total area under the plasma concentration-time curve (AUCₑₓₜᵣₐₚ) of about 1.5% to about 2.5%.

In one embodiment, the antibody, or antigen binding portion thereof, is administered subcutaneously at a dose of about 125 mg to achieve a maximum serum concentration (Cₘₐₓ) of about 2.0 mg/L to about 3.0 mg/L. In one embodiment, the antibody, or antigen binding portion thereof, is administered subcutaneously at a dose of about 125 mg to achieve a dose-normalized serum concentration (C_{max/dose}) of about 0.01 mg/L/mg to 0.03 mg/L/mg. In one embodiment, the antibody, or antigen binding portion thereof, is administered subcutaneously at a dose of about 125 mg to achieve a time to Cₘₐₓ (Tₘₐₓ) of about 4.5 days to about 5.5 days. In one embodiment, the antibody, or antigen binding portion thereof, is administered subcutaneously at a dose of about 125 mg to achieve a time to last measurable serum concentration (Tₗₐₛₜ) of about 8.0 days to about 9.0 days. In one embodiment, the antibody, or antigen binding portion thereof, is administered subcutaneously at a dose of about 125 mg to achieve an area under the plasma concentration-time curve to last measurable serum concentration (AUCₗₐₛₜ) of about 19.0 day*mg/L to about 21.0 day*mg/L. In one embodiment, the antibody, or antigen binding portion thereof, is administered subcutaneously at a dose of about 125 mg to achieve a dose-normalized area under the plasma concentration-time curve from administration to last measurable serum concentration (AUC_{last/dose}) of about 0.15 day*mg/L/mg to about 0.17 day*mg/L/mg.

In one embodiment, the antibody, or antigen binding portion thereof, is administered subcutaneously at a dose of about 250 mg to achieve a maximum serum concentration (Cₘₐₓ) of about 13.0 mg/L to about 13.5 mg/L. In one embodiment, the antibody, or antigen binding portion thereof, is administered subcutaneously at a dose of about 250 mg to achieve a dose-normalized serum concentration (C_{max/dose}) of about 0.04 mg/L/mg to 0.06 mg/L/mg. In one embodiment, the antibody, or antigen binding portion thereof, is administered subcutaneously at a dose of about 250 mg to achieve an apparent volume of distribution (V_{Z}/F) of about 10.0 L to about 11.0 L. In one embodiment, the antibody, or antigen binding portion thereof, is administered subcutaneously at a dose of about 250 mg to achieve a time to Cₘₐₓ (Tₘₐₓ) of about 5.0 days to about 6.0 days. In one embodiment, the antibody, or antigen binding portion thereof, is administered subcutaneously at a dose of about 250 mg to achieve a time to last measurable serum concentration (Tₗₐₛₜ) of about 23 days to about 26 days. In one embodiment, the antibody, or antigen binding portion thereof, is administered subcutaneously at a dose of about 250 mg to achieve a serum concentration half-life (T_{1/2}) of about 3.5 days to about 4.0 days. In one embodiment, the antibody, or antigen binding portion thereof, is administered subcutaneously at a dose of about 250 mg to achieve an area under the plasma concentration-time curve to last measurable serum concentration (AUCₗₐₛₜ) of about 175 day*mg/L to about 185 day*mg/L. In one embodiment, the antibody, or antigen binding portion thereof, is administered subcutaneously at a dose of about 250 mg to achieve a dose-normalized area under the plasma concentration-time curve from administration to last measurable serum concentration (AUC_{last/dose}) of about 0.6 day*mg/L/mg to about 0.8 day*mg/L/mg. In one embodiment, the antibody, or antigen binding portion thereof, is administered subcutaneously at a dose of about 250 mg to achieve an area under the plasma concentration-time curve extrapolated from administration to infinity (AUC_{inf}) of about 145 day*mg/L to about 155 day*mg/L. In one embodiment, subcutaneously at a dose of about 250 mg to achieve a dose-normalized area under the plasma concentration-time curve extrapolated from administration to infinity (AUC_{inf/dose}) of about 0.5 day*mg/L/mg to about 0.7 day*mg/L/mg. In one embodiment, the antibody, or antigen binding portion thereof, is administered subcutaneously at a dose of about 250 mg to achieve an area under the plasma concentration-time curve extrapolated from administration to infinity as a percentage of the total area under the plasma concentration-time curve (AUCₑₓₜᵣₐₚ) of about 1.5% to about 2.5%.

In one embodiment, the antibody, or antigen binding portion thereof, is administered subcutaneously at a dose of about 450 mg to achieve an apparent rate of clearance (CL/F) of about 0.5 L/day to about 1.0 L/day. In one embodiment, the antibody, or antigen binding portion thereof, is administered subcutaneously at a dose of about 450 mg to achieve a maximum serum concentration (Cₘₐₓ) of about 23.0 mg/L to about 25.0 mg/L. In one embodiment, the antibody, or antigen binding portion thereof, is administered subcutaneously at a dose of about 450 mg to achieve a dose-normalized serum concentration (C_{max/dose}) of about 0.04 mg/L/mg to 0.06 mg/L/mg. In one embodiment, the antibody, or antigen binding portion thereof, is administered subcutaneously at a dose of about 450 mg to achieve an apparent volume of distribution (V_{Z}/F) of about 4.0 L to about 6.0 L. In one embodiment, the antibody, or antigen binding portion thereof, is administered subcutaneously at a dose of about 450 mg to achieve a time to Cₘₐₓ (Tₘₐₓ) of about 6.5 days to about 7.5 days. In one embodiment, the antibody, or antigen binding portion thereof, is administered subcutaneously at a dose of about 450 mg to achieve a time to last measurable serum concentration (Tₗₐₛₜ) of about 35 days to about 40 days. In one embodiment, the antibody, or antigen binding portion thereof, is administered subcutaneously at a dose of about 450 mg to achieve a serum concentration half-life (T_{1/2}) of about 4.0 days to about 4.5 days. In one embodiment, the antibody, or antigen binding portion thereof, is administered subcutaneously at a dose of about 450 mg to achieve an area under the plasma concentration-time curve to last measurable serum concentration (AUCₗₐₛₜ) of about 470 day*mg/L to about 480 day*mg/L. In one embodiment, the antibody, or antigen binding portion thereof, is administered subcutaneously at a dose of about 450 mg to achieve a dose-normalized area under the plasma concentration-time curve from administration to last measurable serum concentration (AUC_{last/dose}) of about 0.9 day*mg/L/mg to about 1.1 day*mg/L/mg. In one embodiment, the antibody, or antigen binding portion thereof, is administered subcutaneously at a dose of about 450 mg to achieve an area under the plasma concentration-time curve extrapolated from administration to infinity (AUC_{inf}) of about 570 day*mg/L to about 590 day*mg/L. In one embodiment, the antibody, or antigen binding portion thereof, is administered subcutaneously at a dose of about 450 mg to achieve a dose-normalized area under the plasma concentration-time curve extrapolated from administration to infinity (AUC_{inf/dose}) of about 1.0 day*mg/L/mg to about 1.5 day*mg/L/mg. In one embodiment, the antibody, or antigen binding portion thereof, is administered subcutaneously at a dose of about 450 mg to achieve an area under the plasma concentration-time curve extrapolated from administration to infinity as a percentage of the total area under the plasma concentration-time curve (AUCₑₓₜᵣₐₚ) of about 0.5% to about 1.0%.

In one embodiment, the antibody, or antigen binding portion thereof, is administered subcutaneously at a dose of about 600 mg. In one embodiment, the antibody, or antigen binding portion thereof, is administered subcutaneously at a dose of about 600 mg to achieve a maximum serum concentration (Cₘₐₓ) of about 37.0 mg/L to about 42.0 mg/L. In one embodiment, the antibody, or antigen binding portion thereof, is administered subcutaneously at a dose of about 600 mg to achieve a dose-normalized serum concentration (C_{max/dose}) of about 0.06 mg/L/mg to 0.08 mg/L/mg. In one embodiment, the antibody, or antigen binding portion thereof, is administered subcutaneously at a dose of about 600 mg to achieve an apparent volume of distribution (V_{Z}/F) of about 6.0 L to about 8.0 L. In one embodiment, the antibody, or antigen binding portion thereof, is administered subcutaneously at a dose of about 600 mg to achieve a time to Cₘₐₓ (Tₘₐₓ) of about 8.0 days to about 9.0 days. In one embodiment, the antibody, or antigen binding portion thereof, is administered subcutaneously at a dose of about 600 mg to achieve a time to last measurable serum concentration (Tₗₐₛₜ) of about 47 days to about 52 days. In one embodiment, the antibody, or antigen binding portion thereof, is administered subcutaneously at a dose of about 600 mg to achieve a serum concentration half-life (T_{1/2}) of about 7.0 days to about 7.5 days. In one embodiment, the antibody, or antigen binding portion thereof, is administered subcutaneously at a dose of about 600 mg to achieve an area under the plasma concentration-time curve to last measurable serum concentration (AUCₗₐₛₜ) of about 910 day*mg/L to about 930 day*mg/L. In one embodiment, the antibody, or antigen binding portion thereof, is administered subcutaneously at a dose of about 600 mg to achieve a dose-normalized area under the plasma concentration-time curve from administration to last measurable serum concentration (AUC_{last/dose}) of about 1.4 day*mg/L/mg to about 1.6 day*mg/L/mg. In one embodiment, the antibody, or antigen binding portion thereof, is administered subcutaneously at a dose of about 600 mg to achieve an area under the plasma concentration-time curve extrapolated from administration to infinity (AUC_{inf}) of about 930 day*mg/L to about 950 day*mg/L. In one embodiment, the antibody, or antigen binding portion thereof, is administered subcutaneously at a dose of about 600 mg to achieve a dose-normalized area under the plasma concentration-time curve extrapolated from administration to infinity (AUC_{inf/dose}) of about 1.4 day*mg/L/mg to about 1.6 day*mg/L/mg. In one embodiment, the antibody, or antigen binding portion thereof, is administered subcutaneously at a dose of about 600 mg to achieve an area under the plasma concentration-time curve extrapolated from administration to infinity as a percentage of the total area under the plasma concentration-time curve (AUCₑₓₜᵣₐₚ) of about 1.5% to about 2.0%.

In one embodiment, the antibody, or antigen binding portion thereof, is administered intravenously at a dose of about 3 mg to achieve an area under the plasma concentration-time curve to last measurable serum concentration (AUCₗₐₛₜ) of about 2.0 day*mg/L to about 3.6 day*mg/L. In one embodiment, the antibody, or antigen binding portion thereof, is administered intravenously at a dose of about 3 mg to achieve an area under the concentration-time curve from time zero extrapolated to infinity (AUCₘₐ) of about 2.5 day*mg/L/mg to about 4.0 day*mg/L/mg. In one embodiment, the antibody, or antigen binding portion thereof, is administered intravenously at a dose of about 3 mg to achieve a clearance (CL) of 0.7 to about 1.17 L/day. In one embodiment, the antibody, or antigen binding portion thereof, is administered intravenously at a dose of about 3 mg to achieve a maximum serum concentration (Cₘₐₓ) of about 0.5 mg/L to about 1.1 mg/L. In one embodiment, the antibody, or antigen binding portion thereof, is administered intravenously at a dose of about 3 mg to achieve a volume of distribution of about 3.88 L to about 5.88 L.

In one embodiment, the antibody, or antigen binding portion thereof, is administered intravenously at a dose of about 10 mg to achieve an area under the plasma concentration-time curve to last measurable serum concentration (AUCₗₐₛₜ) of about 10.0 day*mg/L to about 13.0 day*mg/L. In one embodiment, the antibody, or antigen binding portion thereof, is administered intravenously at a dose of about 10 mg to achieve an area under the concentration-time curve from time zero extrapolated to infinity (AUC_{inf}) of about 8.0 day*mg/L/mg to about 13.1 day*mg/L/mg. In one embodiment, the antibody, or antigen binding portion thereof, is administered intravenously at a dose of about 10 mg to achieve a clearance (CL) of 0.7 to about 1.2 L/day. In one embodiment, the antibody, or antigen binding portion thereof, is administered intravenously at a dose of about 10 mg to achieve a maximum serum concentration (Cₘₐₓ) of about 2.1 mg/L to about 3.4 mg/L. In one embodiment, the antibody, or antigen binding portion thereof, is administered intravenously at a dose of about 10 mg to achieve a volume of distribution of about 4.2 L to about 6.3 L.

In one embodiment, the antibody, or antigen binding portion thereof, is administered intravenously at a dose of about 30 mg to achieve an area under the plasma concentration-time curve to last measurable serum concentration (AUCₗₐₛₜ) of about 32.0 day*mg/L to about 48.0 day*mg/L, or an AUCₗₐₛₜ of about 1.3 day*mg/L to about 5.8 day*mg/L. In one embodiment, the antibody, or antigen binding portion thereof, is administered intravenously at a dose of about 30 mg to achieve an area under the concentration-time curve from time zero extrapolated to infinity (AUC_{inf}) of about 31.0 day*mg/L/mg to about 48.0 day*mg/L/mg. In one embodiment, the antibody, or antigen binding portion thereof, is administered intravenously at a dose of about 30 mg to achieve a clearance (CL) of 0.6 to about 1.0 L/day. In one embodiment, the antibody, or antigen binding portion thereof, is administered intravenously at a dose of about 30 mg to achieve a maximum serum concentration (Cₘₐₓ) of about 7 mg/L to about 10.5 mg/L, or a Cₘₐₓ of about 1.7 mg/L to about 3.2 mg/L. In one embodiment, the antibody, or antigen binding portion thereof, is administered intravenously at a dose of about 30 mg to achieve a volume of distribution of about 3.5 L to about 5.5 L.

In one embodiment, the antibody, or antigen binding portion thereof, is administered intravenously at a dose of about 100 mg to achieve an area under the plasma concentration-time curve to last measurable serum concentration (AUCₗₐₛₜ) of about 140 day*mg/L to about 235 day*mg/L, or about 50 day*mg/L to about 105 day*mg/L. In one embodiment, the antibody, or antigen binding portion thereof, is administered intravenously at a dose of about 100 mg to achieve an area under the concentration-time curve from time zero extrapolated to infinity (AUC_{inf}) of about 140 day*mg/L/mg to about 235 day*mg/L/mg, or about 50 day*mg/L to about 106 day*mg/L. In one embodiment, the antibody, or antigen binding portion thereof, is administered intravenously at a dose of about 100 mg to achieve a clearance (CL) of 0.4 to about 0.8 L/day, or about 0.8 to about 2.1 L/day. In one embodiment, the antibody, or antigen binding portion thereof, is administered intravenously at a dose of about 100 mg to achieve a maximum serum concentration (Cₘₐₓ) of about 23 mg/L to about 36 mg/L, or about 12.3 mg/L to about 18.3 mg/L. In one embodiment, the antibody, or antigen binding portion thereof, is administered intravenously at a dose of about 100 mg to achieve a volume of distribution of about 3.5 L to about 5.1 L, or about 5 L to about 7.6 L.

In one embodiment, the antibody, or antigen binding portion thereof, is administered intravenously at a dose of about 300 mg to achieve an area under the plasma concentration-time curve to last measurable serum concentration (AUCₗₐₛₜ) of about 1055 day*mg/L to about 1455 day*mg/L, or an AUCₗₐₛₜ of about 640 day*mg/L to about 910 day*mg/L. In one embodiment, the antibody, or antigen binding portion thereof, is administered intravenously at a dose of about 300 mg to achieve an area under the concentration-time curve from time zero extrapolated to infinity (AUC_{inf}) of about 1000 day*mg/L/mg to about 1475 day*mg/L/mg, or an AUC_{inf} of about 640 day*mg/L to about 910 day*mg/L. In one embodiment, the antibody, or antigen binding portion thereof, is administered intravenously at a dose of about 300 mg to achieve a clearance (CL) of 0.2 to about 0.28 L/day, or about 0.3 L/day to about 0.41 L/day. In one embodiment, the antibody, or antigen binding portion thereof, is administered intravenously at a dose of about 300 mg to achieve a maximum serum concentration (Cₘₐₓ) of about 85 mg/L to about 112 mg/L, or of about 45 mg/L to about 65 mg/L. In one embodiment, the antibody, or antigen binding portion thereof, is administered intravenously at a dose of about 300 mg to achieve a volume of distribution of about 3.0 L to about 4.1 L, or about 4.5 L to about 7 L.

In one embodiment, the antibody, or antigen binding portion thereof, is administered subcutaneously at a dose of about 100 mg to achieve an area under the plasma concentration-time curve to last measurable serum concentration (AUCₗₐₛₜ) of about 50 day*mg/L to about 101 day*mg/L, or of about 5 day*mg/L to about 36 day*mg/L. In one embodiment, the antibody, or antigen binding portion thereof, is administered subcutaneously at a dose of about 100 mg to achieve an area under the concentration-time curve from time zero extrapolated to infinity (AUC_{inf}) of about 55 day*mg/L/mg to about 106 day*mg/L/mg, or of about 7 day*mg/L to about 39 day*mg/L. In one embodiment, the antibody, or antigen binding portion thereof, is administered subcutaneously at a dose of about 100 mg to achieve a clearance (CL) of 0.9 L/day to about 1.8 L/day, or about 0.2 L/day to about 14.4 L/day. In one embodiment, the antibody, or antigen binding portion thereof, is administered subcutaneously at a dose of about 100 mg to achieve a maximum serum concentration (Cₘₐₓ) of about 3.8 mg/L to about 8.1 mg/L, or of about 0.9 mg/L to about 4.7 mg/L. In one embodiment, the antibody, or antigen binding portion thereof, is administered subcutaneously at a dose of about 100 mg to achieve a time to Cₘₐₓ (Tₘₐₓ) of about 2.7 days to about 8.0 days, or of about 1 day to about 6.4 days. In one embodiment, the antibody, or antigen binding portion thereof, is administered subcutaneously at a dose of about 100 mg to achieve a volume of distribution based on the terminal phase (V_{Z}/F) of about 8.6 L to about 16.6 L, or of about 5 L to about 87 L.

In one embodiment, the antibody, or antigen binding portion thereof, is administered subcutaneously at a dose of about 300 mg to achieve an area under the plasma concentration-time curve to last measurable serum concentration (AUCₗₐₛₜ) of about 330 day*mg/L to about 600 day*mg/L, or of about 210 day*mg/L to about 415 day*mg/L. In one embodiment, the antibody, or antigen binding portion thereof, is administered subcutaneously at a dose of about 300 mg to achieve an area under the concentration-time curve from time zero extrapolated to infinity (AUC_{inf}) of about 330 day*mg/L/mg to about 605 day*mg/L/mg, or of about 210 day*mg/L to about 415 day*mg/L. In one embodiment, the antibody, or antigen binding portion thereof, is administered subcutaneously at a dose of about 300 mg to achieve a clearance (CL) of 0.4 to about 1.0 L/day, or of about 0.5 L/day to about 1.7 L/day. In one embodiment, the antibody, or antigen binding portion thereof, is administered subcutaneously at a dose of about 300 mg to achieve a maximum serum concentration (Cₘₐₓ) of about 15 mg/L to about 26 mg/L, or of about 12 mg/L to about 21 mg/L. In one embodiment, the antibody, or antigen binding portion thereof, is administered subcutaneously at a dose of about 300 mg to achieve a time to Cₘₐₓ (Tₘₐₓ) of about 4 days to about 8.5 days, or of about 6.9 days to about 7.2 days. In one embodiment, the antibody, or antigen binding portion thereof, is administered subcutaneously at a dose of about 300 mg to achieve a volume of distribution based on the terminal phase (V_{Z}/F) of about 8.6 L to about 11.6 L, or of about 7 L to about 27 L.

In one aspect, disclosed herein is a method of preventing, managing, or reducing the risk of a venous thromboembolism (VTE), or a complication or symptom associated with VTE following a surgical procedure in a subject. The method includes administering an antibody, or antigen-binding portion thereof, that binds to Factor XI (FXI) to the subject, in which the antibody, or antigen-binding portion thereof, is administered as a single intravenous dose of about 250 mg or a single subcutaneous dose of 300 mg after surgery. The antibody comprises the heavy chain amino acid sequence of SEQ ID NO: 21 and the light chain amino acid sequence of SEQ ID NO: 39. Administration of the antibody, or antigen-binding portion thereof prevents, manages, or reduces the risk of VTE, or the complication or symptom associated with VTE, following the surgical procedure in the subject. In some embodiments, the antibody, or antigen-binding portion thereof is administered within 2 to 24 hours after surgery. In some embodiments, the antibody, or antigen-binding portion thereof is administered as a single intravenous dose of about 250 mg within 12 to 24 hours of surgery. In some embodiments, the antibody, or antigen-binding portion thereof is administered as a single intravenous dose of about 250 mg within 3 hours of surgery. In some embodiments, the antibody, or antigen-binding portion thereof is administered as a single subcutaneous dose of about 300 mg within 12 to 24 hours of surgery. In some embodiments, the antibody, or antigen-binding portion thereof is administered as a single subcutaneous dose of about 300 mg within 2-4 hours of surgery. In some embodiments, the administration of the antibody, or antigen-binding portion thereof does not significantly increase the bleeding risk. In one embodiment, the risk of bleeding in the subject following the administration step is not increased. In some embodiments, the risk of bleeding in the subject following the administration step is not increased as compared to a control, wherein the control is an average measurement of bleeding gathered from a population of subjects undergoing the surgical procedure who have not been administered the antibody, or antigen-binding portion thereof. In some embodiments, the antibody, or antigen-binding portion thereof, comprises a heavy chain variable region (HCVR) comprising a heavy chain complementarity determining region (HCDR) 1, HCDR2, and HCDR3 within the HCVR, wherein the HCVR comprises the amino acid sequence of SEQ ID NO: 23; and a light chain variable region (LCVR) comprising a light chain complementarity determining region (LCDR) 1, LCDR2, and LCDR3 within the LCVR, wherein the LCVR comprises the amino acid sequence of SEQ ID NO: 31.

In one aspect, disclosed herein is a method of preventing, managing, or reducing the risk of a venous thromboembolism (VTE), or a complication or symptom associated with VTE following a surgical procedure in a subject. The method includes administering an antibody, or antigen-binding portion thereof, that binds to Factor XI (FXI) to the subject, in which the antibody, or antigen-binding portion thereof, is administered as a single intravenous dose of about 300 mg 12 to 24 hours after surgery. The antibody comprises the heavy chain amino acid sequence of SEQ ID NO: 1 and the light chain amino acid sequence of SEQ ID NO: 11. Administration of the antibody, or antigen-binding portion thereof prevents, manages, or reduces the risk of VTE, or the complication or symptom associated with VTE, following the surgical procedure in the subject. In some embodiments, the administration of the antibody, or antigen-binding portion thereof does not significantly increase the bleeding risk. In some embodiments, the risk of bleeding in the subject following the administration step is not increased as compared to a control, wherein the control is an average measurement of bleeding gathered from a population of subjects undergoing the surgical procedure who have not been administered the antibody, or antigen-binding portion thereof. In some embodiments, the antibody, or antigen-binding portion thereof, comprises a heavy chain complementarity determining region (HCDR) 1, HCDR2, and HCDR3 within a heavy chain variable region (HCVR) comprising the amino acid sequence of SEQ ID NO: 3; and a light chain complementarity determining region (LCDR) 1, LCDR2, and LCDR3 within a light chain variable region (LCVR) comprising the amino acid sequence of SEQ ID NO: 13.

In any of the above embodiments, the disclosure provides the use of an antibody, or antigen-binding portion thereof that binds to Factor XI (FXI), for the manufacture of a medicament for preventing, managing, or reducing the risk of a venous thromboembolism (VTE), or a complication or symptom associated with VTE following a surgical procedure in a subject.

In one embodiment, the antibody, or antigen-binding portion thereof, is for administration at a dose of about 200 mg to about 600 mg. In one embodiment, the antibody, or antigen-binding portion thereof, comprises a heavy chain complementarity determining region (HCDR) 1, HCDR2, and HCDR3 within a heavy chain variable region (HCVR) comprising the amino acid sequence of SEQ ID NO: 23; and a light chain complementarity determining region (LCDR) 1, LCDR2, and LCDR3 within a light chain variable region (LCVR) comprising the amino acid sequence of SEQ ID NO: 31.

In one embodiment, the antibody, or antigen-binding portion thereof, is administered at a dose of about 200 mg to about 600 mg. In one embodiment, the antibody, or antigen-binding portion thereof, comprises a heavy chain complementarity determining region (HCDR) 1, HCDR2, and HCDR3 within a heavy chain variable region (HCVR) comprising the amino acid sequence of SEQ ID NO: 3; and a light chain complementarity determining region (LCDR) 1, LCDR2, and LCDR3 within a light chain variable region (LCVR) comprising the amino acid sequence of SEQ ID NO: 13.

Other embodiments will become apparent from a review of the ensuing detailed description.

### BRIEF DESCRIPTION OF THE FIGURES

FIG. 1 is a study flow diagram for the REGN7508 study in healthy volunteers described in Example 5.

FIG. 2 is a dose escalation diagram for the REGN7508 study in healthy volunteers described in Example 5.

FIG. 3A is a graph demonstrating dose-dependent effects on functional REGN7508 in plasma in subjects in the intravenous (IV) cohort for the REGN7508 study in healthy volunteers described in Example 5.

FIG. 3B is a graph demonstrating dose-dependent effects on functional REGN7508 in plasma in subjects in the subcutaneous (SC) cohort for the REGN7508 study in healthy volunteers described in Example 5.

FIG. 4A is a graph demonstrating dose-dependent effects on total Factor XI concentration in serum in subjects in the intravenous (IV) cohort for the REGN7508 study in healthy volunteers described in Example 5.

FIG. 4B is a graph demonstrating dose-dependent effects on total Factor XI concentration in serum in subjects in the subcutaneous (SC) cohort for the REGN7508 study in healthy volunteers described in Example 5.

FIG. 5A is a graph demonstrating dose-dependent effects on the percent change from baseline in FXI:C in subjects in the intravenous (IV) cohort for the REGN7508 study in healthy volunteers described in Example 5.

FIG. 5B is a graph demonstrating dose-dependent effects on the percent change from baseline in FXI:C in subjects in the subcutaneous (SC) cohort for the REGN7508 study in healthy volunteers described in Example 5.

FIG. 6 is a graph demonstrating dose-dependent effects on activated partial thromboplastin time (aPTT) prolongation for the REGN7508 study in healthy volunteers described in Example 5. No evidence of PT prolongation was observed.

FIG. 7A is a graph demonstrating dose-dependent effects on activated partial thromboplastin time (aPTT) prolongation in subjects in the intravenous (IV) cohort for the REGN7508 study in healthy volunteers described in Example 5.

FIG. 7B is a graph demonstrating dose-dependent effects on activated partial thromboplastin time (aPTT) prolongation in subjects in the subcutaneous (SC) cohort for the REGN7508 study in healthy volunteers described in Example 5.

FIG. 8A is a graph demonstrating dose-dependent effects on prothrombin time (PT) in subjects in the intravenous (IV) cohort for the REGN7508 study in healthy volunteers described in Example 5. No evidence of PT prolongation was observed.

FIG. 8B is a graph demonstrating dose-dependent effects on prothrombin time (PT) in subjects in the subcutaneous (SC) cohort for the REGN7508 study in healthy volunteers described in Example 5. No evidence of PT prolongation was observed.

FIG. 9A is a scatter plot demonstrating percent change in FXI:C vs. concentration of functional REGN7508 in subjects in the intravenous (IV) cohort for the REGN7508 study in healthy volunteers described in Example 5.

FIG. 9B is a scatter plot demonstrating percent change in FXI:C vs. concentration of functional REGN7508 in subjects in the subcutaneous (SC) cohort for the REGN7508 study in healthy volunteers described in Example 5.

FIG. 10A is a scatter plot demonstrating activated partial thromboplastin time (aPTT) prolongation vs. concentration of functional REGN7508 in subjects in the intravenous (IV) cohort for the REGN7508 study in healthy volunteers described in Example 5.

FIG. 10B is a scatter plot demonstrating activated partial thromboplastin time (aPTT) prolongation vs. concentration of functional REGN7508 in subjects in the subcutaneous (SC) cohort for the REGN7508 study in healthy volunteers described in Example 5.

FIG. 11 is a graph demonstrating dose-dependent effects on functional REGN7508 in plasma and activated partial thromboplastin time (aPTT) prolongation in subjects in the intravenous (IV) cohort for the REGN7508 study in healthy volunteers described in Example 5.

FIG. 12 is a graph demonstrating dose-dependent effects on functional REGN7508 in plasma and activated partial thromboplastin time (aPTT) prolongation in subjects in the subcutaneous (SC) cohort for the REGN7508 study in healthy volunteers described in Example 5.

FIG. 13 is a graph demonstrating dose-dependent effects on activated partial thromboplastin time (aPTT) prolongation for the REGN9933 study in healthy volunteers described in Example 6. No evidence of PT prolongation was observed.

FIGS. 14A and 14B are graphs depicting the mean (+SD) change from baseline in Factor XI activity (FIG. 14A) and aPTT (FIG. 14B) by normal time and dose in healthy volunteers in the study described in Example 6.

FIG. 15 is a graph demonstrating dose-dependent effects on functional REGN9933 in plasma in subjects in the intravenous (IV) and subcutaneous (SC) cohorts for the REGN9933 study in healthy volunteers described in Example 6.

FIG. 16 is a graph demonstrating dose-dependent effects on prothrombin time (PT) in subjects in the intravenous (IV) and subcutaneous (SC) cohorts for the REGN9933 study in healthy volunteers described in Example 6. No evidence of PT prolongation was observed.

FIG. 17 shows the results of the Phase 1 study of gastrointestinal bleeding in patients after administering 75 mg of aspirin alone, or in combination with 250 mg of REGN7508, 300 mg of REGN9933, or 20 mg of Rivaroxaban.

FIG. 18 is a study flow diagram for the REGN7508 study in subjects having an elective, unilateral, total knee arthroplasty (TKA) described in Example 8.

FIG. 19 depicts that Phase 2 studies in venous thromboembolism after orthopedic surgery confirm the effectiveness of REGN 7508.

FIG. 20 is a study flow diagram for the REGN9933 study in subjects having an elective, unilateral, total knee arthroplasty (TKA) described in Example 9.

FIG. 21 depicts that Phase 2 studies in venous thromboembolism after orthopedic surgery confirm the effectiveness of REGN 7508.

FIG. 22 is a graph demonstrating concentrations of functional REGN9933 in plasma and total REGN9933 in serum by nominal time in participants undergoing total knee arthroplasty (TKA) as described in Example 9.

FIG. 23 is a graph demonstrating the change from baseline in aPTT by nominal time and treatment group in participants undergoing total knee arthroplasty (TKA) as described in Example 9.

FIG. 24 is a graph demonstrating the prothrombin time (PT) by nominal time and treatment group in participants undergoing total knee arthroplasty (TKA) as described in Example 9.

FIG. 25 is a graph demonstrating the percent change from baseline in Factor XI functional activity (FXI:C) by nominal time and treatment group in participants undergoing total knee arthroplasty (TKA) as described in Example 9.

FIGS. 26A and 26B show a comparative study of the efficiency of inhibition of REGN7508^{CAT} and REGN9933^{A2} to inhibit intrinsic-mediated coagulation activity, while exerting no effect on prothrombin time (PT) clotting function but causing moderate-to-minimal reduction in extrinsic-mediated thrombin generation in human plasma in vitro. FIG. 26A shows the changes in activated partial thromboplastin time (aPTT) and FIG. 26B shows PT compared to baseline (shown as ratio) were determined; the average change of duplicate samples is plotted for each concentration tested in human plasma.

FIG. 27A is a panel of graphs demonstrating observed (points) and predicted (solid lines with 90% prediction intervals, shaded) activated partial thromboplastin time (aPTT) versus time for selected REGN7508^{CAT} IV and SC doses (30 mg intravenous (IV), 125 mg subcutaneous (SC), 450 mg SC, 600 mg SC) in healthy volunteers (HVs). Dashed vertical lines indicate the time of dose administration as described in Example 12.

FIG. 27B is a panel of graphs demonstrating observed (points) and predicted (solid lines with 90% prediction intervals, shaded) activated partial thromboplastin time (aPTT) versus time for selected REGN9933^{A2} IV and SC doses (30 mg intravenous (IV), 100 mg IV, 100 mg subcutaneous (SC), 300 mg SC) in healthy volunteers (HVs). Dashed vertical lines indicate the time of dose administration as described in Example 12.

FIG. 28A is a graph demonstrating a simulated activated partial thromboplastin time (aPTT) fold change from baseline using healthy volunteer (HV) model parameters at steady state (Q2W SC) for REGN7508^{CAT} as described in Example 12. Shaded areas represent 90% prediction intervals.

FIG. 28B is a graph demonstrating a simulated activated partial thromboplastin time (aPTT) fold change from baseline using HV model parameters at steady state (Q2W SC) for REGN9933^{A2} as described in Example 12. Shaded areas represent 90% prediction intervals.

FIG. 28C is a graph demonstrating simulated activated partial thromboplastin time (aPTT) fold change from baseline at 100% increased clearance for REGN7508^{CAT} as described in Example 12. Shaded areas represent 90% prediction intervals.

FIG. 28D is a graph demonstrating simulated aPTT fold change from baseline at 100% increased clearance for REGN9933^{A2} as described in Example 12. Shaded areas represent 90% prediction intervals.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs.

As used herein the term “about” when used in reference to a particular recited numerical value, means that the value may vary from the recited value by no more than 10%. For example, the expression “about 100” includes 90 and 110, and all values in between (e.g., 91, 92, 93, etc.).

Although any methods and materials similar or equivalent to those described herein can be used in the practice or testing of the present disclosure, the preferred methods and materials are now described. All patents, applications and non-patent publications mentioned in this specification are incorporated herein by reference in their entireties.

### DEFINITIONS

The expression “FXI,” and the like, also known as “coagulation factor XI” or “Factor XI”, refers to the human plasma serine protease (unless designated as being from another species) comprising the amino acid sequence as set forth in amino acid residues 19 through 625 of accession number NP_000119.1 (SEQ ID NO: 41). Human FXI containing a myc-myc-hexahistidine tag is shown as SEQ ID NO: 42 (with amino acid residues 1-607 being human FXI and amino acid residues 608-635 being the myc-myc-hexahistidine tag).

In certain instances, cell lines were prepared that expressed the FXI proteins, subunits of the FXI protein, and chimera proteins containing one or more FXI subunits, tag sequences, and plasma kallikrein protein sequences. For example, SEQ ID NO: 43 (construct hFXI_PKA1) is a chimera containing, at amino acids 1-85, the apple 1 domain (PKA1) of human kallikrein B1 (amino acids G20-C104 of human kallikrein B1 [SEQ ID NO: 48]), at amino acids 86-606, amino acids H105-V625 of human FXI (hFXI), and at amino acids 607-634, the myc-myc-hexahistidine tag.

For example, SEQ ID NO: 44 (construct hFXI_PKA2) is a chimera containing, at amino acids 1-90, amino acids E19-S108 of hFXI, at amino acids 91-174, the apple 2 domain (PKA2, also referred to as “A2”) of hKLKB1 (amino acids C111-C193 SEQ ID NO: 48), at amino acids 175-605, amino acids A195-V625 of hFXI, and at amino acids 606-633, the myc-myc-hexahistidine tag.

For example, SEQ ID NO: 45 (construct hFXI_PKA3) is a chimera containing, at amino acids 1-180, amino acids E19-L198 of hFXI, at amino acids 181-264, the apple 3 domain (PKA3) of hKLKB1 (amino acids C201-C284 SEQ ID NO: 48), at amino acids 265-605, amino acids H285-V625 of hFXI, and at amino acids 606-633, the myc-myc-hexahistidine tag.

For example, SEQ ID NO: 46 (construct hFXI_PKA4) is a chimera containing, at amino acids 1-271, amino acids E19-V289 of hFXI, at amino acids 272-355, the apple 4 domain (PKA4) of hKLKB1 (amino acids C292-C375 SEQ ID NO: 48), at amino acids 356-605, amino acids M376-V625 of hFXI, and at amino acids 606-633, the myc-myc-hexahistidine tag.

For example, SEQ ID NO: 47 (construct hKLKB1.mmh) is a chimera containing, at amino acids 1-619, amino acids G20-A638 of hKLKB1, and at amino acids 620-647, the myc-myc-hexahistidine tag.

As used herein, the expression “anti-FXI antibody” includes both monovalent antibodies with a single specificity, as well as bispecific antibodies comprising a first arm that binds FXI and a second arm that binds a second (target) antigen, wherein the anti-FXI arm comprises any of the HCVR/LCVR or CDR sequences as set forth in Table 1 herein. The expression “anti-FXI antibody” also includes antibody-drug conjugates (ADCs) comprising an anti-FXI antibody or antigen-binding portion thereof conjugated to a drug or toxin (i.e., cytotoxic agent). The expression “anti-FXI antibody” also includes antibody-radionuclide conjugates (ARCs) comprising an anti-FXI antibody or antigen-binding portion thereof conjugated to a radionuclide.

The term “anti-FXI antibody,” as used herein, means any antigen-binding molecule or molecular complex comprising at least one complementarity determining region (CDR) that specifically binds to or interacts with FXI or a portion of FXI or an epitope within FXI. Non-limiting examples of anti-FXI antibody include REGN7508 and REGN9933, which are described below. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-FXI antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full length antibody molecules. The terms “antigen-binding portion” of an antibody, “antigen-binding fragment” of an antibody, and the like, as used herein, include any enzymatically obtainable, synthetic, or genetically-engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and, optionally, constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present disclosure using routine techniques available in the art.

In certain instances, it may be desirable to antagonize FXI, for example, for inhibiting the formation of blood clots. The antibodies for use in the disclosed methods act as antagonist antibodies, which serve as inhibitors of FXI or FXIa activity and concomitantly serve as inhibitors of intrinsic pathway thrombosis/clot formation. The antibodies for use in the disclosed methods may function by preventing the interaction between FXI and its upstream activators coagulation factor XII (FXII) and/or coagulation faction II (FII or thrombin). The antibodies for use in the disclosed methods may also function by preventing the interaction between FXI and its downstream target coagulation factor IX (FIX). The antibodies for use in the disclosed methods may also function by sequestering FXI from the blood stream of a patient.

The term “human antibody,” as used herein, is intended to include non-naturally occurring human antibodies. The term includes antibodies that are recombinantly produced in a non-human mammal, or in cells of a non-human mammal. The term is not intended to include antibodies isolated from or generated in a human subject.

The antibodies for use in the disclosed methods may, in some embodiments, be recombinant and/or non-naturally occurring human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. In certain embodiments, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant disclosure encompasses antibodies having one or more mutations in the hinge, C_{H}2 or C_{H}3 region, which may be desirable, for example, in production, to improve the yield of the desired antibody form.

The term “specifically binds,” or “binds specifically to,” or the like, means that an antibody, or antigen-binding fragment thereof, forms a complex with an antigen that is relatively stable under physiologic conditions. Specific binding can be characterized by an equilibrium dissociation constant of at least about 1×10⁻⁶ M or less (i.e., a smaller K_{D} denotes a tighter binding). Methods for determining whether two molecules specifically bind are well known in the art and include, for example, equilibrium dialysis, surface plasmon resonance, and the like. As described herein, antibodies have been identified by surface plasmon resonance, e.g., BIACORE™, which bind specifically to FXI. Moreover, multi-specific antibodies that bind to FXI protein and one or more additional antigens or a bi-specific that binds to two different regions of FXI are nonetheless considered antibodies that “specifically bind,” as used herein.

The antibodies for use in the disclosed methods may be isolated antibodies. An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present disclosure. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The anti-FXI antibodies for use in the disclosed methods may comprise one or more amino acid substitutions, insertions and/or deletions in the framework and/or CDR regions of the heavy and light chain variable domains. Such mutations can be readily ascertained by comparing the amino acid sequences for use in the disclosed methods to sequences available from, for example, public antibody sequence databases. Once obtained, antibodies and antigen-binding fragments that contain one or more mutations can be easily tested for one or more desired property such as, improved binding specificity, increased binding affinity, improved or enhanced antagonistic or antagonistic biological properties (as the case may be), reduced immunogenicity, etc. Antibodies and antigen-binding fragments obtained in this general manner are encompassed within for use in the methods of the present disclosure.

The present disclosure also includes anti-FXI antibodies for use in the disclosed methods comprising variants of any of the HCVR, LCVR, and/or CDR amino acid sequences disclosed herein having one or more conservative substitutions. For example, the present disclosure includes anti-FXI antibodies for use in the disclosed methods having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set forth in Table 1 herein.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or Gap, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs Gap or BestFit using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, GCG software contains programs such as Gap and BestFit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the disclosure to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

The term post-surgical “venous thrombosis,” as used herein, refers to a condition that occurs when a blood clot, also known as a thrombus, forms in a vein following surgery or a surgical procedure in a subject.

The term post-surgical “venous thromboembolism” or “VTE,” as used herein, refers to the obstruction of a vein caused by a thrombus, or blood clot, that occurs following surgery or a surgical procedure in a subject. VTE comprises deep vein thrombosis (DVT) and pulmonary embolism (PE), depending on the location of the obstruction.

As used herein, the term “subject” refers to a mammal (e.g., human, rat, mouse, cat, dog, cow, sheep, horse, goat, rabbit), preferably a human, for example, in need of prevention, management, reducing the risk of, and/or treatment of a venous thromboembolism and/or or complications or symptoms associated with it. The subject may have been diagnosed with, predisposed to, or is at an elevated risk of developing venous thromboembolism, and/or symptoms associated with it. In some embodiments, the subject may have recently had a surgical procedure. In many embodiments, the term “subject” may be used interchangeably with the term “patient.”

Various clinical risk factors increase the risk of developing post-surgical VTE including, but not limited to: age, gender, obesity, diabetes, blood clotting disorders or a history of blood clots, malignant disease, surgery/trauma, pregnancy/puerperium, spinal cord injury, and contraceptive use/hormone intake.

As used herein, the terms “treat,” “treating,” or the like, mean to alleviate complications and/or symptoms, eliminate the causation of symptoms either on a temporary or permanent basis, and/or to increase duration of survival of the subject.

As used herein, the terms “prevent,” “prevention,” or the like, mean to reduce the risk, e.g., of thrombosis or blood clot formation following a surgery or a surgical procedure in a subject without substantially increasing bleeding risk. Further, they mean to stop or hinder the complications or symptoms or causation of complications or symptoms either on a temporary or permanent basis, and/or to increase duration of survival of the subject. With respect to post-surgical venous thromboembolism, the phrase “prevent post-surgical venous thromboembolism” may refer to stopping the formation of a thrombus, or blood clot in the vein, from forming in a subject, or decreasing the amount or rate of venous thrombus by at least 10% in a subject who has recently had a surgical procedure or where a person is predisposed to, or is at an elevated risk of developing post-surgical venous thromboembolism. In one embodiment, the phrase “prevent post-surgical VTE” refers to stopping the formation of a thrombus, or blood clot in the vein, from forming in a subject who has recently had a surgical procedure and/or is predisposed to, or at an elevated risk of developing post-surgical venous thromboembolism, by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, at least about 98%, at least about 99%, at least about 10% to about 99%, at least about 25% to about 99%, at least about 50% to about 99%, at least about 75% to about 99% as compared to an untreated individual who underwent the same surgery or surgical procedure.

As used herein, the term “managing the risk of venous thromboembolism (VTE)” refers to administering an effective amount of an anticoagulant or anti-FXI antibody, or an antigen-binding fragment thereof, to a subject identified as being at increased risk of VTE following a surgical procedure, thereby reducing the incidence or severity of VTE without substantially increasing bleeding risk.

As used herein the term “without substantially increasing bleeding risk” or “does not substantially increase bleeding risk” means that administration of the therapeutic agent does not increase the risk of bleeding beyond a predefined threshold, such as no more than about 0%, 5%, 10%, 20%, 30%, 40%, or 50% increase in bleeding events, or no measurable prolongation of prothrombin time (PT) relative to baseline or control. In some embodiments, the risk of bleeding in the subject following the administration step is not increased as compared to subjects undergoing the surgical procedure who have not been administered the antibody, or antigen-binding portion thereof. In some embodiments, the risk of bleeding in the subject following the administration step is not increased as compared to a control, in which the control is an average measurement of bleeding gathered from a population of subjects undergoing the surgical procedure who have not been administered the antibody, or antigen-binding portion thereof.

As used herein, the term “after the surgical procedure” refers to any time period following completion of a surgical procedure, including immediately after the surgical wound closure and extending through the post-operative recovery phase, during which the subject remains at risk of venous thromboembolism (VTE) or related complications. In some embodiments, “after the surgical procedure” refers to a time about 1-36 hours after the surgical wound closure. In some embodiments, “after the surgical procedure” refers to a time about: 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 30 hours, 32 hours, or 36 hours after the surgical wound closure. In some embodiments, “after the surgical procedure” refers to a time between 1 and 2 hours, between 2 and 3 hours, between 3 and 4 hours, between 4 and 5 hours, between 5 and 6 hours, between 6 and 7 hours, between 7 and 8 hours, between 8 and 9 hours, between 9 and 10 hours, between 10 and 11 hours, between 11 and 12 hours, between 12 and 13 hours, between 13 and 14 hours, between 14 and 15 hours, between 15 and 16 hours, between 16 and 17 hours, between 17 and 18 hours, between 18 and 19 hours, between 19 and 20 hours, between 20 and 21 hours, between 21 and 22 hours, between 22 and 23 hours, between 23 and 24 hours, between 24 and 30 hours, or between 30 and 36 hours after the surgical wound closure.

As used herein the term “complication” or “symptom” associated with post-surgical venous thromboembolism include, but is not limited to post-thrombotic syndrome, e.g., pain, swelling, discoloration, and scaling in the affected limb; chronic thromboembolic pulmonary hypertension; chronic venous insufficiency (can occur when a blood clot heals and turns into scar tissue, damaging the valves in the veins); phlegmasia cerulea dolens, lower extremity swelling, warmth, redness, pain, shortness of breath, chest pain exacerbated by inspiration, hemoptysis, bleeding at the surgical site or elsewhere, or a combination of one or more complications and/or symptoms.

The terms “surgery” and “surgical procedure” are used interchangeably in this document. Non limiting examples of surgery include orthopedic, abdominal, cardiac, thoracic surgery, vascular, trauma, abdominopelvic, gynecologic, urologic, bariatric, plastic, reconstructive, otolaryngic, arthroscopic, organ transplant, or neurologic. Non-limiting examples of orthopedic surgery include total knee arthroplasty (TKA), total hip arthroplasty, spinal surgery, shoulder surgery, surgery of the upper or lower limbs, and foot and hand surgery. Non-limiting examples of organ transplants include transplant of heart, kidney, liver, and lung. Venous thromboembolism (VTE), including deep vein thrombosis (DVT) and pulmonary embolism (PE), is a major complication following organ transplantation. It arises from surgical stress, inflammation, immunosuppressive medications such as steroids and calcineurin inhibitors, and pre-existing risk factors. These conditions promote clot formation in veins, which can compromise the transplanted organ or other sites, increasing morbidity, mortality, and post-operative complications.

As used herein, the expression “in combination with” means that a first agent, e.g., a first antibody or antigen-binding fragment thereof, is administered before, after, or concurrent with a second therapeutic or prophylactic agent, e.g., a second antibody or antigen-binding fragment thereof, or other agent. The term “in combination with” also includes sequential or concomitant administration of a first agent, e.g., a FXI antibody or antigen-binding fragment thereof and a second agent.

The term “contraindication to anticoagulation,” as used herein, indicates that the subject or patient exhibits one or more conditions that could increase the risks associated with anticoagulation therapies, e.g., active bleeding; severe bleeding diathesis; recent, planned or emergency high bleeding-risk surgery/procedure; major trauma; acute intracranial hemorrhage; etc.

The term “poor venous access,” as used herein, refers to the difficulty or inability to successfully insert a needle into a vein to draw blood or administer medications and fluids. This condition can be due to various factors, including, e.g.: small or fragile veins; veins that are difficult to locate or palpate; veins that collapse easily; previous damage or scarring from frequent venipuncture or intravenous therapy; patient conditions such as obesity, dehydration, or chronic illness; etc.

The term “excessive intraoperative blood loss” during a surgical procedure is subjective and depends on the judgment of the surgeon. Typically, excessive intra-operative blood loss refers to the loss of a significant amount of blood that exceeds the expected or normal amount for the specific type of surgery being performed. Complications of excessive intra-operative blood loss may include hemodynamic instability and may require interventions such as blood transfusions or other measures to control bleeding and maintain adequate blood volume and pressure.

The term “normal INR,” as used herein, is defined as defined as <1.1.

The term “normal aPTT,” as used herein, may be based on reference range of local laboratory. The upper end of aPPT normal range typically is between 35 and 40 seconds depending on the local laboratory.

### Characteristics of FXI Antibodies

The present disclosure includes methods of use for anti-FXI antibodies that bind FXI. In some embodiments, such antibodies bind FXI with a K_{D} of less than about 500 pM as measured by surface plasmon resonance at 25 °C, or at 37 °C. According to certain embodiments, the disclosure includes anti-FXI antibodies that bind human FXI with a K_{D} of less than about 400 pM, less than about 300 pM, less than about 200 pM, less than about 150 pM, less than about 100 pM, less than about 80 pM, less than about 50 pM, less than about 40 pM, less than about 30 pM, less than about 20 pM, less than about 10 pM, less than about 5 pM, less than about 3 pM, or less than about 1 pM.

The present disclosure includes methods for use of anti-FXI antibodies that bind activated human FXI (FXIa) with a K_{D} of less than about 1,000 pM as measured by surface plasmon resonance at 25 °C, or at 37 °C. According to certain embodiments, the disclosure includes anti-FXI antibodies that bind human FXI with a K_{D} of less than about 900 pM, less than about 800 pM, less than about 700 pM, less than about 500 pM, less than about 250 pM, less than about 100 pM, less than about 80 pM, less than about 50 pM, less than about 40 pM, less than about 30 pM, less than about 20 pM, less than about 10 pM, less than about 5 pM, less than about 3 pM, or less than about 1 pM.

The present disclosure includes methods of use for anti-FXI antibodies that bind human FXI with a dissociative half life (t½) of greater than about 10 minutes as measured by surface plasmon resonance at 25 °C, or 37 °C. According to certain embodiments, the disclosure includes anti-FXI antibodies that bind human FXI with a t½ of greater than about 20 minutes, greater than about 50 minutes, greater than about 100 minutes, greater than about 120 minutes, greater than about 150 minutes, greater than about 300 minutes, greater than about 350 minutes, greater than about 400 minutes, greater than about 450 minutes, greater than about 500 minutes, greater than about 550 minutes, greater than about 600 minutes, greater than about 700 minutes, greater than about 800 minutes, greater than about 900 minutes, greater than about 1000 minutes, greater than about 1100 minutes, or greater than about 1200 minutes.

The present disclosure includes methods of use for anti-FXI antibodies that bind human FXIa with a dissociative half life (t½) of greater than about 10 minutes as measured by surface plasmon resonance at 25 °C, or 37 °C. According to certain embodiments, the disclosure includes anti-FXI antibodies that bind human FXI with a t½ of greater than about 20 minutes, greater than about 50 minutes, greater than about 100 minutes, greater than about 120 minutes, greater than about 150 minutes, greater than about 300 minutes, greater than about 350 minutes, greater than about 400 minutes, greater than about 450 minutes, greater than about 500 minutes, greater than about 550 minutes, greater than about 600 minutes, greater than about 700 minutes, greater than about 800 minutes, greater than about 900 minutes, greater than about 1000 minutes, greater than about 1100 minutes, or greater than about 1200 minutes.

The present disclosure includes methods of use for anti-FXI antibodies that may or may not bind non-human FXI. As used herein, an antibody “does not bind” a particular antigen (e.g., monkey, mouse or rat FXI if the antibody, when tested in an antigen binding assay such as surface plasmon resonance exhibits a K_{D} of greater than about 1000 nM, or does not exhibit any antigen binding, in such an assay. Another assay format that can be used to determine whether an antibody binds or does not bind a particular antigen, according to this aspect of the disclosure, is ELISA.

It is generally known in the art that activated FXI (FXIa) activates Factor IX by selectively cleaving arg-ala and arg-val peptide bonds. Factor IXa, in turn, forms a complex with Factor VIIIa (FIXa-FVIIIa) and activates Factor X. The present disclosure includes anti-FXI antibodies that inhibit FXI-mediated activation of human FX in plasma by at least about 85% with an IC₅₀ of less than about 100 pM. Using an assay format familiar to one of skill in the art, an IC₅₀ value can be calculated as the concentration of antibody required to activate FXI-mediated signaling to the half-maximal signal observed. Thus, according to certain embodiments, the disclosure includes anti-FXI antibodies that mediate human FXI-mediated activation of human FX in plasma by at least about 85% with an IC₅₀ of less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, or less than about 5 pM, as measured using an assay format familiar to one of skill in the art.

The present disclosure also includes methods of use for anti-FXI antibodies that inhibit FXIa-mediated activation of human FX in plasma by at least about 25% with an IC₅₀ of less than about 50 pM. Using an assay format familiar to one of skill in the art, an IC₅₀ value can be calculated as the concentration of antibody required to activate FXIa-mediated signaling to the half-maximal signal observed. Thus, according to certain embodiments, the disclosure includes anti-FXI antibodies that mediate human FXIa-mediated activation of human FX in plasma by at least about 25% with an IC₅₀ of less than about 200 pM, less than about 300 pM, less than about 200 pM, less than about 100 pM, less than about 50 pM, less than about 40 pM, less than about 30 pM, less than about 20 pM, less than about 10 pM, less than about 9 pM, less than about 8 pM, less than about 7 pM, less than about 6 pM, less than about 5 pM, less than about 4 pM, less than about 3 pM, less than about 2 pM, or less than about 1 pM, as measured using the assay format familiar to one of skill in the art.

The present disclosure includes methods of use for anti-FXI antibodies that preferentially bind to the apple 2 domain (A2) as demonstrated by direct binding to A2 domain constructs or by competing with one or more specific A2-binding antibodies. In one embodiment, the antibodies, or antigen-binding fragments thereof, disclosed herein do not bind the A2 domain of FXI. In one embodiment, the antibodies, or antigen-binding fragment thereof, preferentially bind to the catalytic domain (CAT) as demonstrated by direct binding to CAT domain constructs or by competing with one or more specific CAT-binding antibodies. In one embodiment, the antibodies, or antigen-binding fragments thereof, disclosed herein do not bind the catalytic domain of FXI.

The present disclosure includes methods of use for anti-FXI antibodies that prolong the activated partial thromboplastin time (aPTT), which is a measure of intrinsic pathway thrombogenesis, while having no measurable effect on prothrombin time (PT), which is a measure of extrinsic pathway thrombogenesis, in human plasma. In one embodiment, the aPTT is measured in pooled human plasma treated with ellagic acid and the PT is measured in pooled human plasma treated with tissue factor using a hemostasis analyzer. It is generally known in the art that ellagic acid stimulates the intrinsic pathway of thrombogenesis in vitro and tissue factor stimulates the extrinsic pathway of thrombogenesis. Here, the anti-FXI antibody prolongs aPTT about two-fold at a concentration of less than 100 nM, less than 90 nM, less than 80 nM, less than 70 nM, less than 60 nM, less than 50 nM, less than 40 nM, less than 30 nM, less than 20 nM, less than 10 nM, 1 nM-100 nM, 1 nM-100 nM, 1 nM-50 nM, 100 pM-50 nM, 5 nM-50 nM, 5 nM-40 nM, 5 nM-15 nM, 10 nM-20 nM, 15 nM-25 nM, 20 nM-30 nM, 25 nM-35 nM, 30 nM-40 nM, 35 nM-45 nM, 40 nM-50 nM, 45 nM-55 nM, 50 nM-60 nM, 55 nM-65 nM, 60 nM-100 nM, 65 nM-75 nM, 70 nM-80 nM, 75 nM-85 nM, 80 nM-90 nM, 85 nM-95 nM, 90 nM-100 nM, or 95 nM-105 nM, without doubling PT.

The present disclosure includes methods of use for anti-FXI antibodies that inhibit the production of thrombin via the intrinsic pathway (intrinsic thrombin) in human plasma in vitro with little to no effect on the production of thrombin via the extrinsic pathway (extrinsic thrombin). In one embodiment, pathway-specific thrombin production is determined in vitro by a thrombin generation assay using a calibrated automated thrombogram. Here, a thrombin generation profile is generated and peak thrombin concentration is determined in ellagic acid treated plasma and in tissue factor treated plasma with and without an anti-FXI antibody. Thus, in one embodiment, anti-FXI antibodies inhibit the production of intrinsic thrombin at a concentration of about 0.1 nM-100 nM, 1 nM-100 nM, 5 nM-500 nM, 5 nM-100 nM, 10 nM-100 nM, 10 nM-50 nM, 5 nM-15 nM, 10 nM-20 nM, 25 nM-35 nM, 30 nM-40 nM, 35 nM-45 nM, 40 nM-50 nM, 45 nM-55 nM, 50 nM-60 nM, 55 nM-65 nM, 60 nM-65 nM, ≥20 nM, ≥25 nM, ≥30 nM, ≥35 nM, ≥40 nM, ≥45 nM, ≥50 nM, ≥55 nM, ≥5 nM, ≥10 nM, or ≥15 nM. Here, the anti-FXI antibodies have no effect on the production of extrinsic thrombin with any concentration up to 500 nM.

The present disclosure includes methods of use for anti-FXI antibodies that increase by at least two-fold activated partial thromboplastin time (aPTT) in a clinical trial subject in vivo without measurably affecting prothrombin time (PT). Here, a primate is administered an anti-FXI antibody, plasma is obtained from the primate, the plasma is contacted with ellagic acid or tissue factor, and then the aPTT or PT respectively is determined in an assay format familiar to one of skill in the art. In one embodiment, the primate is a human. In one embodiment, the primate is a monkey.

In one embodiment, the anti-FXI antibody REGN9933 or REGN7508, alone or in combination, is administered parenterally (e.g., intravenously, intravitreally, intraocularly, intracutaneously, subcutaneously, intramuscularly, etc.) at a dose of about 0.01 mg/kg-20 mg/kg, 0.1 mg/kg-10 mg/kg, 1 mg/kg-10 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, or about 15 mg/kg.

In one embodiment, the aPTT is prolonged with the anti-FXI treatment relative to no anti-FXI treatment at least 1.5-fold, at least 2-fold, at least 2.5-fold, at least 3-fold, at least 3.5-fold, at least 4-fold, at least 4.5-fold, at least 5-fold, at least 5.5-fold, or at least 6-fold.

In one embodiment, the anti-FXI-mediated aPTT prolongation effect persists in the subject after receiving a dose of the anti-FXI antibody for at least 1 week, at least 2 weeks, at least 3 weeks, at least 4 weeks, at least 5 weeks, at least 6 weeks, at least 7 weeks, at least 8 weeks, at least 3 months, at least 4 months, at least 5 months, or at least 6 months.

The present disclosure includes methods of use for anti-FXI antibodies that inhibit intrinsic pathway peak thrombin activity in a clinical trial subject in vivo without measurably affecting extrinsic pathway peak thrombin activity. Here, a clinical trial subject is administered an anti-FXI antibody, plasma is obtained from the subject, the plasma is contacted with ellagic acid or tissue factor, and then the generation of intrinsic thrombin or extrinsic thrombin respectively is determined in a thrombin generation assay familiar to one of skill in the art.

In one embodiment, the anti-FXI antibody is administered parenterally at a dose of 0.01 mg/kg-20 mg/kg, 0.1 mg/kg-10 mg/kg, 1 mg/kg-10 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, or about 15 mg/kg. In one embodiment, the anti-FXI antibody REGN9933 or REGN7508, alone or in combination, is administered parenterally (e.g., intravenously or subcutaneously) at a dose of about 3 mg, 5 mg, 10 mg, 15 mg, 30 mg, 60 mg, 100 mg, 125 mg, 200 mg, 250 mg, 300 mg, 450 mg, 600 mg. In one embodiment, a single dose of the anti-FXI antibody REGN9933 or REGN7508, alone or in combination, is administered parenterally (e.g., intravenously or subcutaneously) at a dose of about 3 mg, 5 mg, 10 mg, 15 mg, 30 mg, 60 mg, 100 mg, 125 mg, 200 mg, 250 mg, 300 mg, 450 mg, 600 mg before, during, or after surgery.

In one embodiment, the peak intrinsic thrombin (i.e., the thrombin generated via ellagic acid) activity in the anti-FXI treatment relative to no anti-FXI treatment is inhibited by about 1%-100%, 5%-95%, 10%-90%, 20%-80%, 1%-10%, 5%-20%, 10%-30%, 15%-40%, 20%-50%, 25%-60%, 5%-15%, 10%-20%, 15%-25%, 20%-30%, 25%-35%, 30%-40%, 35%-45%, 40%-50%, 45%-55%, 50%-60%, 55%-65%, 60%-70%, 65%-75%, 70%-80%, 75%-85%, 80%-90%, 85%-95%, 90%-100%, 95%-105%, or ≥100%.

In one embodiment, the anti-FXI-mediated inhibition of peak intrinsic thrombin activity persists in the subject after receiving a dose of the anti-FXI antibody for at least 1 week, at least 2 weeks, at least 3 weeks, at least 4 weeks, at least 5 weeks, at least 6 weeks, at least 7 weeks, at least 8 weeks, at least 3 months, at least 4 months, at least 5 months, or at least 6 months.

A binding characteristic of an antibody of the disclosure (e.g., any of the binding characteristics mentioned herein above), when disclosed in term of being “measured by surface plasmon resonance” means that the relevant binding characteristic pertaining to the interaction between the antibody and the antigen are measured using a surface plasmon resonance instrument (e.g., a Biacore® instrument, GE Healthcare) using standard Biacore® assay conditions, or a substantially similar assay, familiar to one of skill in the art. In certain embodiments, the binding parameters are measured at 25 °C, while in other embodiments, the binding parameters are measured at 37 °C.

The present disclosure includes methods of use for antibodies or antigen-binding fragments thereof that specifically bind FXI, comprising an HCVR and/or an LCVR comprising an amino acid sequence selected from any of the HCVR and/or LCVR amino acid sequences listed in Table 1.

The antibodies for use in the disclosed methods may possess one or more of the aforementioned biological characteristics, or any combination thereof. The foregoing list of biological characteristics of the antibodies of the disclosure is not intended to be exhaustive. Other biological characteristics of the antibodies of the present disclosure will be evident to a person of ordinary skill in the art from a review of the present disclosure including the working Examples herein.

### Epitope Mapping and Related Technologies

The epitope to which the antibodies for use in the disclosed methods bind may consist of a single contiguous sequence of 3 or more (e.g., 3, 4, 5, 6, 7, 8, 9, 10, 11, 12, 13, 14, 15, 16, 17, 18, 19, 20 or more) amino acids of a FXI protein. Alternatively, the epitope may consist of a plurality of non-contiguous amino acids (or amino acid sequences) of FXI. In some embodiments, the epitope is located on or near a surface of FXI, for example, in the domain that interacts with any one of its ligands, e.g., FXIIa, thrombin, and FIX. In other embodiments, the epitope is located on or near a surface of FXI that does not interact with the FXI ligand, e.g., at a location on the surface of FXI at which an antibody, when bound to such an epitope, does not interfere with the interaction between FXI and its ligand.

The present disclosure includes methods for use of anti-FXI antibodies that bind to the same epitope as any of the specific exemplary antibodies described herein (e.g., antibodies comprising any of the amino acid sequences as set forth in Table 1 herein). Likewise, the present disclosure also includes methods for use of anti-FXI antibodies that compete for binding to FXI with any of the specific exemplary antibodies described herein (e.g., antibodies comprising any of the amino acid sequences as set forth in Table 1 herein).

### Preparation of Human Antibodies

In certain embodiments, it may be desirable to test anti-human FXI antibodies in mice or rats that have been engineered to express a human FXI receptor. These mice or rats may be beneficial in circumstances wherein the anti-FXI antibodies may only bind human FXI, but will not cross react with mouse or rat FXI. Any method known to those skilled in the art may be used for generating such FXI humanized mice and rats.

In general, the antibodies of the instant disclosure possess very high affinities, typically possessing K_{D} of from about 10⁻¹² through about 10⁻⁹ M, when measured by binding to antigen either immobilized on solid phase or in solution phase.

### Bioequivalents

The anti-FXI antibodies and antibody fragments for use in the methods of the present disclosure encompass proteins having amino acid sequences that vary from those of the described antibodies but that retain the ability to bind human FXI. Such variant antibodies and antibody fragments comprise one or more additions, deletions, or substitutions of amino acids when compared to parent sequence, but exhibit biological activity that is essentially equivalent to that of the described antibodies. Likewise, the anti-FXI antibody-encoding DNA sequences of the present disclosure encompass sequences that comprise one or more additions, deletions, or substitutions of nucleotides when compared to the disclosed sequence, but that encode an anti-FXI antibody or antibody fragment that is essentially bioequivalent to an anti-FXI antibody or antibody fragment for use in the disclosed methods. Examples of such variant amino acid and DNA sequences are discussed above.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of anti-FXI antibodies for use in the disclosed methods may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antibodies may include anti-FXI antibody variants comprising amino acid changes which modify the glycosylation characteristics of the antibodies, e.g., mutations which eliminate or remove glycosylation.

### Therapeutic Formulations and Administration

The disclosure provides methods for use of pharmaceutical compositions comprising the anti-FXI antibodies or antigen-binding fragments thereof of the present disclosure. The pharmaceutical compositions of the disclosure are formulated with suitable carriers, excipients, and other agents that provide improved transfer, delivery, tolerance, and the like. A multitude of appropriate formulations can be found in the formulary known to pharmaceutical chemists: Remington's Pharmaceutical Sciences, Mack Publishing Company, Easton, PA.

The dose of antibody administered to a patient may vary depending upon the age and the size of the patient, target disease, conditions, route of administration, and the like. The preferred dose is typically calculated according to body weight or body surface area. In an adult patient, it may be advantageous to parenterally (e.g., intravenously, intravitreally, intraocularly, intracutaneously, subcutaneously, intramuscularly, etc.) administer the antibody of the present disclosure normally at a single dose of about 0.01 to about 20 mg/kg body weight, more preferably about 0.02 to about 7, about 0.03 to about 5, or about 0.05 to about 3 mg/kg body weight. In one embodiment, the anti-FXI antibody is administered to an individual at a dose of about 3 mg, 5 mg, 10 mg, 15 mg, 30 mg, 60 mg, 100 mg, 125 mg, 200 mg, 250 mg, 300 mg, 450 mg, 600 mg. In one embodiment, REGN7508 or REGN9933 or their antigen binding fragment is administered to an individual parenterally (e.g., intravenously, intravitreally, intraocularly, intracutaneously, subcutaneously, intramuscularly, etc.). In one embodiment, a single dose of the anti-FXI antibody REGN9933 or REGN7508, alone or in combination, is administered parenterally (e.g., intravenously or subcutaneously) at a dose of about 3 mg, 5 mg, 10 mg, 15 mg, 30 mg, 60 mg, 100 mg, 125 mg, 200 mg, 250 mg, 300 mg, 450 mg, 600 mg before, during, or after surgery. In some embodiments, the individual underwent surgery prior to administration of REGN7508, REGN9933, or a combination of both. The frequency and the duration of the treatment can be adjusted. Effective dosages and schedules for administering anti-FXI antibodies may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

The injectable preparations may include dosage forms for intravenous, intravitreal, intraocular, subcutaneous, intracutaneous and intramuscular injections, drip infusions, etc. These injectable preparations may be prepared by methods publicly known.

### Therapeutic Uses of the Antibodies

The present disclosure includes methods comprising administering to a subject in need thereof a therapeutic composition comprising an anti-FXI antibody (e.g., an anti-FXI antibody comprising any of the HCVR/LCVR or CDR sequences as set forth in Table 1 herein), or an antigen-binding fragment thereof. The therapeutic composition can comprise any one or more of the anti-FXI antibodies or antigen-binding fragments thereof disclosed herein, and a pharmaceutically-acceptable carrier or diluent.

The present disclosure includes methods to treat, prevent, manage, or reduce the occurrence of post-surgical venous thromboembolism, while managing or minimizing the risk of bleeding by administering to a patient in need of such treatment an anti-FXI antibody, or antigen-binding fragment thereof, as disclosed elsewhere herein. Non-limiting examples of surgery include orthopedic, abdominal, cardiac, thoracic surgery. Non-limiting examples of orthopedic surgery include total knee arthroplasty, total hip arthroplasty, shoulder surgery.

In the context of the methods of treatment described herein, the anti-FXI antibody may be administered as a monotherapy (i.e., as the only therapeutic agent) or in combination with one or more additional therapeutic agents.

### Combination Therapies and Formulations

The present disclosure includes methods comprising administration of compositions and therapeutic formulations comprising any of the anti-FXI antibodies described herein in combination with one or more additional therapeutically active components to subjects in need thereof to a subject post-surgery. In one embodiment, the anti-FXI antibody REGN7508, or an antigen-binding fragment thereof, is administered as a monotherapy post-surgery. In one embodiment, the anti-FXI antibody REGN9933, or an antigen-binding fragment thereof, is administered as a monotherapy post-surgery. In one embodiment, the anti-FXI antibody REGN7508, or an antigen-binding fragment thereof, is administered in combination with the anti-FXI antibody REGN9933, or an antigen-binding fragment thereof post-surgery. Non-limiting examples of the combination of REGN7508, or an antigen-binding fragment thereof and REGN9933, or an antigen-binding fragment thereof are shown in the table below.

| REGN7508, | |
|---|---|
| or its antigen- | |
| binding fragment | REGN9933, or its antigen-binding fragment |
| about 3 mg | about 3 mg, 5 mg, 10 mg, 15 mg, 30 mg, 60 mg, |
| | 100 mg, 125 mg, 200 mg, 250 mg, 300 mg, 450 |
| | mg, or 600 mg |
| about 5 mg | about 3 mg, 5 mg, 10 mg, 15 mg, 30 mg, 60 mg, |
| | 100 mg, 125 mg, 200 mg, 250 mg, 300 mg, 450 |
| | mg, or 600 mg |
| about 10 mg | about 3 mg, 5 mg, 10 mg, 15 mg, 30 mg, 60 mg, |
| | 100 mg, 125 mg, 200 mg, 250 mg, 300 mg, 450 |
| | mg, or 600 mg |
| about 30 mg | about 3 mg, 5 mg, 10 mg, 15 mg, 30 mg, 60 mg, |
| | 100 mg, 125 mg, 200 mg, 250 mg, 300 mg, 450 |
| | mg, or 600 mg |
| about 60 mg | about 3 mg, 5 mg, 10 mg, 15 mg, 30 mg, 60 mg, |
| | 100 mg, 125 mg, 200 mg, 250 mg, 300 mg, 450 |
| | mg, or 600 mg |
| about 100 mg | about 3 mg, 5 mg, 10 mg, 15 mg, 30 mg, 60 mg, |
| | 100 mg, 125 mg, 200 mg, 250 mg, 300 mg, 450 |
| | mg, or 600 mg |
| about 125 mg | about 3 mg, 5 mg, 10 mg, 15 mg, 30 mg, 60 mg, |
| | 100 mg, 125 mg, 200 mg, 250 mg, 300 mg, 450 |
| | mg, or 600 mg |
| about 200 mg | about 3 mg, 5 mg, 10 mg, 15 mg, 30 mg, 60 mg, |
| | 100 mg, 125 mg, 200 mg, 250 mg, 300 mg, 450 |
| | mg, or 600 mg |
| about 250 mg | about 3 mg, 5 mg, 10 mg, 15 mg, 30 mg, 60 mg, |
| | 100 mg, 125 mg, 200 mg, 250 mg, 300 mg, 450 |
| | mg, or 600 mg |
| about 300 mg | about 3 mg, 5 mg, 10 mg, 15 mg, 30 mg, 60 mg, |
| | 100 mg, 125 mg, 200 mg, 250 mg, 300 mg, 450 |
| | mg, or 600 mg |
| about 450 mg | about 3 mg, 5 mg, 10 mg, 15 mg, 30 mg, 60 mg, |
| | 100 mg, 125 mg, 200 mg, 250 mg, 300 mg, 450 |
| | mg, or 600 mg |
| about 600 mg | about 3 mg, 5 mg, 10 mg, 15 mg, 30 mg, 60 mg, |
| | 100 mg, 125 mg, 200 mg, 250 mg, 300 mg, 450 |
| | mg, or 600 mg |

In one embodiment of the above combination, REGN7508, or its antigen-binding fragment is administered intravenously, and REGN9933 or its antigen-binding fragment is administered subcutaneously. In another embodiment of the above combination, both REGN7508, or its antigen-binding fragment and REGN9933 or its antigen-binding fragment are administered subcutaneously. In one embodiment of the above combination, REGN7508, or its antigen-binding fragment is administered subcutaneously and REGN9933 or its antigen-binding fragment is administered intravenously. In another embodiment of the above combination, both REGN7508, or its antigen-binding fragment and REGN9933 or its antigen-binding fragment are administered intravenously.

In some embodiments, the anti-FXI antibodies for use in the disclosed methods may be co-formulated with one or more drugs used to treat or prevent thrombosis associated with recent trauma or surgery.

In some embodiments, REGN7508, REGN9933 or their antigen-binding fragments may be used simultaneously, together with, or co-administered with other anti-coagulants. In some embodiments, REGN7508, REGN9933 or their antigen-binding fragments may be administered as a single dose, and the other anti-coagulants may be used daily for a defined period of time. In some embodiments, non-limiting examples of anti-coagulants include aspirin, enoxaparin, apixaban, warfarin, rivaroxaban, edoxaban, betrixaban, and dabigatran. In one embodiment, REGN7508, or an antigen-binding fragment thereof, is administered with enoxaparin. In one embodiment, REGN7508, or an antigen-binding fragment thereof, is administered with apixaban. In one embodiment, REGN7508, or an antigen-binding fragment thereof, is administered with rivaroxaban. In one embodiment, REGN7508, or an antigen-binding fragment thereof, is administered with aspirin. In one embodiment, REGN9933, or an antigen-binding fragment thereof, is administered with enoxaparin. In one embodiment, REGN9933, or an antigen-binding fragment thereof, is administered with apixaban. In one embodiment, REGN9933, or an antigen-binding fragment thereof, is administered with rivaroxaban. In one embodiment, REGN9933, or an antigen-binding fragment thereof, is administered with aspirin. In some embodiments, REGN7508, REGN9933 or their antigen-binding fragments may be co-administered with other anti-coagulants.

In some embodiments, the combination of REGN7508 or its antigen-binding fragments and other anti-coagulants may be used in exemplary non-limiting doses and dosing frequencies.

| REGN7508, | |
|---|---|
| or its antigen- | |
| binding fragment | Other anti-coagulant |
| 250 mg IV or 300 | Enoxaparin 40 mg SC injection daily for about 7-14 |
| mg SC injection | days after surgery |
| 250 mg IV or 300 | Apixaban 2.5 mg, oral, once or twice daily for about |
| mg SC injection | 7-14 days after surgery |
| 250 mg IV or 300 | Rivaroxaban, 15 mg or 20 mg oral, once daily for |
| mg SC injection | about 7-14 days after surgery |
| 250 mg IV or 300 | Aspirin, 75 mg oral, once daily for about 7-14 days |
| mg SC injection | after surgery |

In some embodiments, the combination of REGN9933 or its antigen-binding fragments and other anti-coagulants may be used in exemplary non-limiting doses.

| REGN9933, | |
|---|---|
| or its antigen- | |
| binding fragment | Other anti-coagulant |
| 300 mg IV or 300 | Enoxaparin 40 mg SC injection daily for about 7-14 |
| mg SC injection | days after surgery |
| 300 mg IV or 300 | Apixaban 2.5 mg, oral, once or twice daily for about |
| mg SC injection | 7-14 days after surgery |
| 300 mg IV or 300 | Rivaroxaban, 15 mg or 20 mg oral, once daily for |
| mg SC injection | about 7-14 days after surgery |
| 300 mg IV or 300 | Aspirin, 75 mg oral, once daily for about 7-14 days |
| mg SC injection | after surgery |

The anti-FXI antibodies for use in the disclosed methods may also be administered and/or co-formulated in combination with antivirals, antibiotics, analgesics, antioxidants, COX inhibitors, and/or non-steroidal anti-inflammatory drugs (NSAIDs).

In one embodiment, the anti-FXI antibodies for use in the disclosed methods may also be administered and/or co-formulated in combination with a therapeutically effective amount of lipid lowering compounds.

In one embodiment, the anti-FXI antibodies for use in the disclosed methods may also be administered and/or co-formulated in combination with a therapeutically effective amount of NSAIDS. Non-limiting examples of NSAIDs include naproxen, diclofenac, ibuprofen, and celecoxib.

In one embodiment, the anti-FXI antibodies for use in the disclosed methods may also be administered and/or co-formulated in combination with a therapeutically effective amount of steroidal anti-inflammatory drugs. Non-limiting examples of steroidal anti-inflammatory drugs include prednisone, cortisone, hydrocortisone, methylprednisone, betamethasone, and prednisolone.

The present disclosure includes methods comprising administration of additional therapeutically active component(s), e.g., any of the agents listed above or derivatives thereof, may be administered just prior to, concurrent with, or shortly after the administration of an anti-FXI antibody; (for purposes of the present disclosure, such administration regimens are considered the administration of an anti-FXI antibody “in combination with” an additional therapeutically active component). The present disclosure includes methods of administration of a pharmaceutical composition in which an anti-FXI antibody is co-formulated with one or more of the additional therapeutically active component(s) as described elsewhere herein.

### Administration Regimens

According to certain embodiments of the present disclosure, multiple doses of an anti-FXI antibody (or a pharmaceutical composition comprising a combination of an anti-FXI antibody and any of the additional therapeutically active agents mentioned herein) may be administered to a subject over a defined time course. The methods according to this aspect of the disclosure comprise sequentially administering to a subject multiple doses of an anti-FXI antibody of the disclosure. As used herein, “sequentially administering” means that each dose of anti-FXI antibody is administered to the subject at a different point in time, e.g., on different days separated by a predetermined interval (e.g., hours, days, weeks, or months). The present disclosure includes methods which comprise sequentially administering to the patient a single initial dose of an anti-FXI antibody, followed by one or more secondary doses of the anti-FXI antibody, and optionally followed by one or more tertiary doses of the anti-FXI antibody.

The terms “initial dose,” “secondary doses,” and “tertiary doses,” refer to the temporal sequence of administration of the anti-FXI antibody for use in the disclosed methods. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of anti-FXI antibody, but generally may differ from one another in terms of frequency of administration. In certain embodiments, however, the amount of anti-FXI antibody contained in the initial, secondary, and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In certain exemplary embodiments of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of anti-FXI antibody, which is administered to a patient prior to the administration of the very next dose in the sequence with no intervening doses.

The methods according to this aspect of the disclosure may comprise administering to a patient any number of secondary and/or tertiary doses of an anti-FXI antibody. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient. The administration regimen may be carried out indefinitely over the lifetime of a particular subject, or until such treatment is no longer therapeutically needed or advantageous.

In embodiments involving multiple secondary doses, each secondary dose may be administered at the same frequency as the other secondary doses. For example, each secondary dose may be administered to the patient 1 to 2 weeks or 1 to 2 months after the immediately-preceding dose. Similarly, in embodiments involving multiple tertiary doses, each tertiary dose may be administered at the same frequency as the other tertiary doses. For example, each tertiary dose may be administered to the patient 2 to 12 weeks after the immediately-preceding dose. In certain embodiments of the disclosure, the frequency at which the secondary and/or tertiary doses are administered to a patient can vary over the course of the treatment regimen. The frequency of administration may also be adjusted during the course of treatment by a physician depending on the needs of the individual patient following clinical examination.

The present disclosure includes administration regimens in which 2 to 6 loading doses are administered to a patient at a first frequency (e.g., once a week, once every two weeks, once every three weeks, once a month, once every two months, etc.), followed by administration of two or more maintenance doses to the patient on a less frequent basis. For example, according to this aspect of the disclosure, if the loading doses are administered at a frequency of once a month, then the maintenance doses may be administered to the patient once every six weeks, once every two months, once every three months, etc.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25 °C, and pressure is at or near atmospheric. Additional details regarding subject anti-FXI monoclonal antibodies including REGN9933 and REGN7508 can be found, for example, in PCT/US2023/036946 and PCT/US2022/078530, herein incorporated by reference.

### Example 1: Generation of Human Antibodies to FXI

Human antibodies to FXI were generated as previously described in International Application No. PCT/2022/78530, filed on Oct. 21, 2022 and published as WO2023/070097; and in International Application No. PCT/US2023/36946, filed on Nov. 7, 2023, and published as WO2024/102369; the entire contents of each of which are expressly incorporated herein by reference.

Briefly, antibodies were generated in a mouse comprising DNA encoding human immunoglobulin heavy and kappa light chain variable regions. In one embodiment, the human antibodies were generated in a VELOCIMMUNE® mouse. In one embodiment, VelocImmune® (VI) mice were immunized with human FXI. The antibody immune response was monitored by FXI specific immunoassay. For example, sera were assayed for specific antibody titers to purified full-length FXI. Antibody-producing clones were isolated using both B-cell Sorting Technology (BST) and hybridoma methods. For example, when a desired immune response was achieved, splenocytes were harvested and fused with mouse myeloma cells to preserve their viability and form hybridoma cell lines. The hybridoma cell lines were screened and selected to identify cell lines that produce FXI-specific antibodies.

Anti-FXI antibodies were also isolated directly from antigen-positive mouse B cells without fusion to myeloma cells, as described in U.S. Pat. No. 7,582,298, herein specifically incorporated by reference in its entirety. Using this method, several fully human anti-FXI antibodies (i.e., antibodies possessing human variable domains and human constant domains) were obtained.

The biological properties of the exemplary antibody generated in accordance with the methods of this Example, controls, and comparators are described in detail in the Examples set forth below.

### Example 2: Heavy and Light Chain Variable Region Sequences

Table 1, columns 2 and 4 set forth the amino acid sequence identifiers of the heavy and light chain variable regions and CDRs of exemplary anti-FXI antibodies of the disclosure. The corresponding nucleic acid sequence identifiers for the exemplary anti-FXI antibodies of the disclosure is set forth in Table 1, columns 3 and 5.

TABLE 1
Anti-FXI Antibody Sequence Identifiers

| | REGN9933 | REGN9933 | REGN7508 | REGN7508 |
|---|---|---|---|---|
| | Amino Acid | Nucleic Acid | Amino Acid | Nucleic Acid |
| Antibody Part | SEQ ID NO | SEQ ID NO | SEQ ID NO | SEQ ID NO |
| Full Length | 1 | 2 | 21 | 22 |
| Heavy Chain | | | | |
| (HC) | | | | |
| Heavy Chain | 3 | 4 | 23 | 24 |
| Variable | | | | |
| Region | | | | |
| (HCVR) | | | | |
| HCDR1 | 5 | 6 | 25 | 26 |
| HCDR2 | 7 | 8 | 27 | 28 |
| HCDR3 | 9 | 10 | 29 | 30 |
| Full Length | 11 | 12 | 39 | 40 |
| Light Chain | | | | |
| (LC) | | | | |
| Light Chain | 13 | 14 | 31 | 32 |
| Variable | | | | |
| Region | | | | |
| (LCVR) | | | | |
| LCDR1 | 15 | 16 | 33 | 34 |
| LCDR2 | 17 | 18 | 35 | 36 |
| LCDR3 | 19 | 20 | 37 | 38 |

SEQ ID NO: 17 comprises the following amino acid sequence: AAS.

SEQ ID NO: 18 comprises the following nucleotide sequence: GCTGCATCC.

SEQ ID NO: 35 comprises the following amino acid sequence: DAS.

SEQ ID NO: 36 comprises the following nucleotide sequence: GATGCATCC.

The exemplary full length anti-FXI antibody contains fully human Fc gamma 4 heavy chain (i.e., IgG4 Fc) and fully human light chain sequences. However, as will be appreciated by a person of ordinary skill in the art, an antibody having a particular Fc isotype can be converted to an antibody with a different Fc isotype (e.g., an antibody with a mouse IgG1 Fc can be converted to an antibody with a human IgG4, etc.), but in any event, the variable domains (including the CDRs)—which are indicated by the numerical identifiers shown in Table 1—will remain the same, and the binding properties to antigen are expected to be identical or substantially similar regardless of the nature of the Fc domain.

### Example 3: Tissue Cross-reactivity in Normal Human and Cynomolgus Monkey Tissues

A complex of unlabeled REGN9933 and biotinylated F(ab′)2 donkey anti-human IgG, FcG fragment-specific antibody (DkaHuIgG) was prepared via overnight incubation of the antibodies in a 1:1.5 ratio. The prepared complex was applied to cryosections of normal human and cynomolgus monkey tissues (≥3 donors per tissue, where available) at REGN9933 concentrations of 2 g/mL and g/mL (3 g/mL and 15 g/mL biotinylated DkaHuIgG, respectively). In addition, the test article was substituted with a human IgG4k antibody (HuIgG4) as an isotype control.

REGN9933 produced staining of the positive control material (FXI UV-resin spot slides) at both staining concentrations. REGN9933 did not specifically react with the negative control material (human hypercalcemia of malignancy peptide, amino acid residues 1-34, UV resin spot slides) at either staining concentration. The control article, HuIgG4, did not specifically react with either the positive or negative control materials. There also was no staining of the assay control slides. The specific reactions of REGN9933 in all staining runs with the positive control material and the lack of specific reactivity with the negative control material, as well as the lack of reactivity of the control article, indicated that the assay was sensitive, specific, and reproducible. No binding was present with REGN9933 in the human or cynomolgus monkey normal tissue panels examined.

### Example 4: Studies in Cynomolgus Monkeys

A series of studies were conducted in cynomolgus monkeys to assess pharmacokinetics (PK), pharmacodynamics (PD), toxicokinetics (TK), and toxicology for subject anti-FXI monoclonal antibodies.

### REGN9933

For single-dose, pharmacokinetic studies, REGN9933 was administered by intravenous (IV) (0.5, 5, 30 mg/kg) or subcutaneous (SC) (30 mg/kg) administration to female animals. For repeat-dose toxicokinetic studies, REGN9933 was administered once weekly by IV (100 mg/kg) or SC (5, 30, 100 mg/kg REGN9933) administration for 5 weeks; once weekly by IV (30, 100 mg/kg) injections for 13 weeks; once weekly SC (30, 100 mg/kg) injections for 26 weeks to young animals; or once weekly SC (100 mg/kg) for 26 weeks to sexually mature animals.

For repeat-dose toxicology studies, REGN9933 was administered once weekly by SC (5, 30, 100 mg/kg) or IV (100 mg/kg) administration for 5 weeks, followed by a 13-week dose-free recovery period; once weekly IV (30, 100 mg/kg) infusion for 13 weeks to male and female young adult animals, followed by a 12-week dose-free recovery period; or once weekly SC (30, 100 mg/kg) injection for 26 weeks to male and female animals, young adult or sexually mature, followed by a 12-week dose-free recovery period.

### REGN7508

For repeat-dose toxicology studies, REGN7508 was administered once weekly SC (5, 30, 100 mg/kg) or IV (100 mg/kg) administration for 13 weeks to male and female animals, age 1.9 to 3.5 years and weighing between 1.6 and 2.6 kg at initiation of dosing, followed by a 12-week dose-free recovery period; or once weekly SC (100 mg/kg) injection for 26 weeks to male and female animals, young adult or sexually mature, followed by a 12-week dose-free recovery period.

### Safety Evaluation

For both 13-week studies and the 26-week study, complete necropsies were performed at the end of the dosing period and at the end of the recovery period. REGN9933 was well tolerated, with no unscheduled deaths and no adverse effects evident. There were no drug-related clinical observations or changes in qualitative food consumption, body weight and body-weight changes, body temperature, or in the ophthalmology parameters. There were no REGN9933-related effects on safety pharmacology parameters (heart rate and blood pressure, qualitative echocardiogram (ECG) waveforms, respiratory rate, blood oxygen saturation, and neurologic assessments), clinical pathology (hematology and urinalysis) nor anatomical pathology changes (gross necropsy findings, organ weight changes, or microscopic findings) that were considered related to REGN9933 administration; nor was occult blood detected in fecal samples.

No apparent impact of anti-drug antibodies (ADA) on REGN9933 concentration-time profiles was observed in studies of cynomolgus monkeys.

### Evaluation of Total FXI Levels in Plasma

Concentrations of total subject monoclonal antibody REGN9933 or REGN7508 (all drug, independent of the presence of target) were measured in serum using an Enzyme-linked Immunosorbent Assay (ELISA) with a mouse anti-human IgG Fc as the capture monoclonal antibody and a biotinylated anti-human Ig kappa light chain specific as the detection monoclonal antibody. A NeutrAvidin conjugated with horseradish peroxidase (NeutrAvidin-HRP) was used to convert a luminol-based substrate into a signal proportional to the concentration of the total FXI monoclonal antibody concentration. Total FXI (REGN9933- or REGN7508-bound and free) levels were measured in plasma using the Affinity Biologicals Factor XI ELISA kit (FXI-AG) (Ancaster, ON). The manufacturer's protocol was followed for determining FXI concentrations.

The PK profiles of total REGN9933 were as expected in the different cynomolgus monkey studies. Specifically, after administration of a single dose, the profile characterized by target-mediated clearance at low concentrations and linear, dose-proportional kinetics at high concentrations. After administration of repeat doses, concentration-time profiles of total REGN9933 were characterized by a brief distribution phase, followed by a linear elimination phase consistent with saturation of the TMC pathway during the treatment period.

For all studies, peak concentrations (Cₘₐₓ) increased in an approximately dose-proportional manner. After single administration, a greater than dose proportional increase in exposure (area under the concentration-time curve [AUC]) was observed across all dose groups, consistent with nonlinear kinetics that is more pronounced at low doses and concentrations, and total body clearance was dose-dependent. Throughout the 5 week repeat-dose treatment, exposure AUC calculated during the dosing interval (AUCtau) increased in an approximately dose proportional manner. During the 13-week recovery period, dose-normalized exposure (AUC_{rec}/Dose) values increased with increasing SC dose by 1.7-fold (increasing 1.2-fold from low-to-mid dose and 1.3-fold from mid-to-high dose), suggesting a minor degree of nonlinearity consistent with TMC observed at the lower REGN9933 concentrations. Accumulation was observed across all dose groups and routes of administration for both 5-week and 13-week repeat dose treatments.

For all studies, after administration of REGN9933, concentrations of soluble FXI in plasma increased all dose groups (IV and SC) compared to pre-dose levels, indicating a certain degree of dose response, but there was no general correlation with the PK of REGN9933 when comparing individual profiles in the same animals.

### Ex Vivo Evaluation of Coagulation

The capacity of REGN9933 or REGN7508 to block coagulation pathways in plasma from animals was assessed using clotting assays and thrombin generation assays (TGAs). Ex vivo clotting-time assays were performed using plasma from blood samples collected over the duration of the study. Blood sample collections were performed with the anticoagulant sodium citrate at a concentration of about 3.5%, which interferes less with the performance of coagulation assays than other anticoagulants (e.g., EDTA). Clotting time was determined using the Diagnostica Stago START 4 Hemostasis Analyzer (Diagnostica Stago, Parsippany, NJ) to measure the activated partial thromboplastin time (aPTT) that measures clotting activated by an intrinsic pathway activator (ellagic acid [EA]) and Prothrombin Time (PT) that measures clotting activated by an extrinsic pathway activator (tissue factor [TF]). The times to clotting were reported as fold change to baseline. Thrombin generation was measured on a Stago Diagnostica Calibrated Automated Thrombogram (CAT) (Diagnostica Stago, Parsippany, NJ). The thrombin generation assay (TGA) was conducted with the intrinsic pathway activator ellagic acid (EA) or the extrinsic pathway activator tissue factor (TF). Data for TGA are reported as percentage change from baseline for the following parameters: Lag time, peak thrombin concentration and total thrombin concentration.

In all dose and recovery studies (5 weeks then 13 weeks; 13 weeks then 12 weeks, 26 weeks then 12 weeks), administration of REGN9933 and REGN7508 resulted in prolongation of aPTT of 2- to 3-fold in both young adult and sexually mature animals. Values for aPTT remained prolonged after the recovery period but a decrease in magnitude was observed. Prothrombin time at all postdose collections in REGN9933-treated animals was comparable to control.

### Example 5: Randomized, Double-Blind, Placebo-Controlled Study to Evaluate the Safety, Tolerability, Pharmacokinetics, and Pharmacodynamics of Single Ascending Doses of REGN7508 in Healthy Adult Subjects

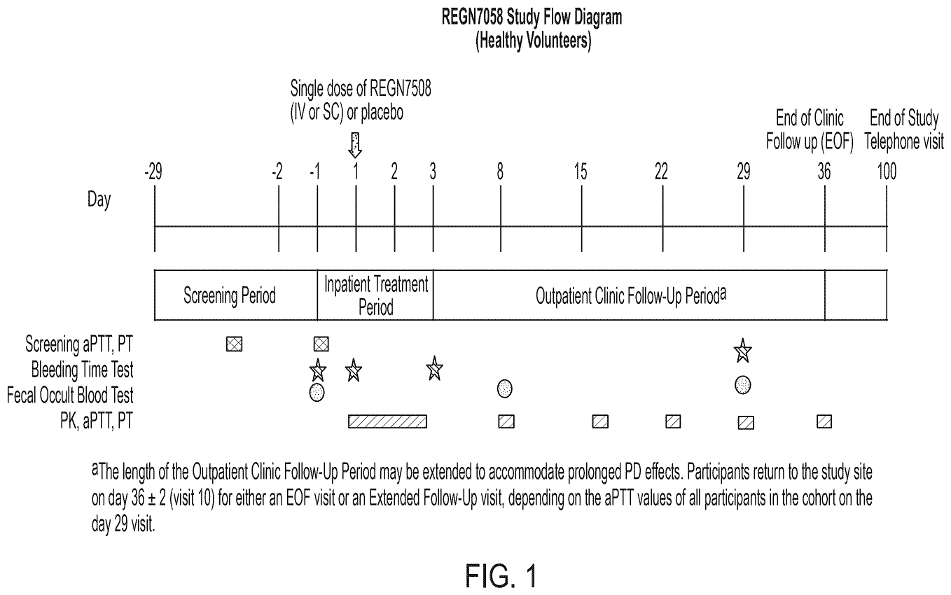

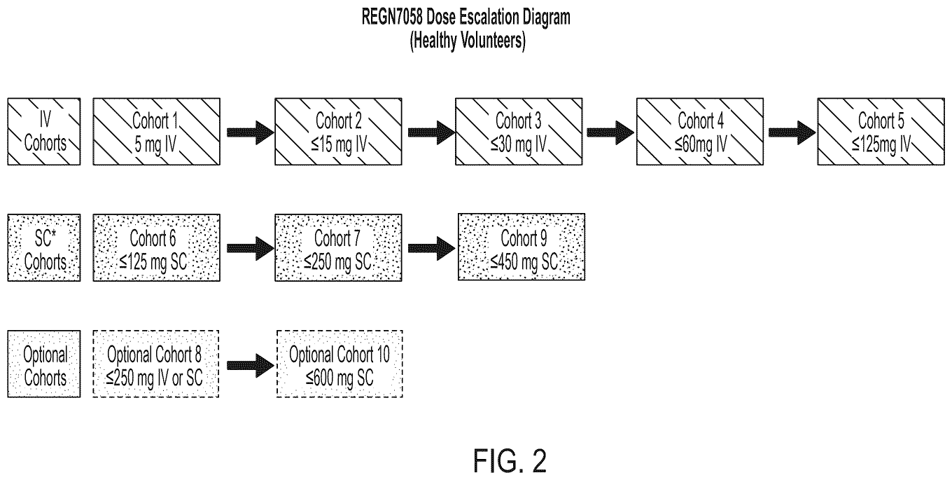

This randomized, double-blind, placebo-controlled, single-center study was designed to evaluate the safety, tolerability, PK, and PD of single ascending IV and SC doses of the human mAb REGN7508 in healthy adults (FIG. 1). The study design using sentinel dosing, sequential cohorts, and planned review of emerging safety and any available PK and PD data allowed for progressing through multiple dose levels expeditiously yet safely (FIG. 2).

The study population comprised healthy male and permanently amenorrheic female participants between 18 and 55 years who are at low risk for bleeding and thrombosis. Advanced age is a risk factor for both bleeding and clotting, so a very conservative age range was chosen for adult participants (Li, 2017) (Yousufuddin, 2019). Women capable of menstruation were excluded since the literature reports that some FXI-deficient women may experience heavy menstruation (menorrhagia) (Kadir, 1999). Participants requiring medications associated with bleeding risk, including nonsteroidal anti-inflammatory drugs (NSAIDs), were excluded. Each participant was required to have a normal PT, aPTT, and platelet count, a negative FOBT, and normal BTT prior to randomization to ensure bleeding risk is as low as possible before receiving study drug. All of these design elements were included to limit the risk of bleeding in this FIH study.

The primary objective was to evaluate the safety and tolerability of single doses of REGN7508 in healthy participants. Safety was assessed by the incidences and severities of TEAEs. In addition, several assessments were included in this study to monitor and explore bleeding risk. Prothrombin time is a standard and validated measure of extrinsic/common pathway activation. Elevated PT following administration of anticoagulation therapies inhibiting the extrinsic/common pathway is associated with an elevated bleeding risk (Woodruff, 2018).

Bleeding time tests and FOBTs were also included in this study as exploratory endpoints and additional assessments of bleeding. Bleeding time tests were measured on day 1 and day 3 to assess bleeding times during anticipated maximal concentration of REGN7508 in the IV and SC cohorts, respectively, and a postdose stool sample was collected no sooner than 3 days after dose administration (to allow for gastrointestinal transit time of red blood cells) but no later than day 8. Flexibility in the timing of stool collection was provided to accommodate each volunteer's habits. Additionally, BTTs and FOBTs were measured at the week 4 follow-up visit to assess any evidence of bleeding risk after prolonged exposure to the study drug. It was anticipated there would be no observed changes in PT, bleeding time, or fecal occult blood in this study, as no changes in these parameters were seen in preclinical studies of REGN7508. Isolated abnormal BTTs and FOBTs are not considered to be actionable safety measures due to poor positive predictive value for clinically significant bleeding events and the inter- and intra-participant variability (Gewirtz, 1996) (Urbas, 2017).

The PD endpoint aPTT is a standard clinically validated measure of intrinsic/common pathway activation. Normal aPTT values are generally between 30 and 40 seconds. In preclinical PK/PD studies in NHPs, REGN7508 increased aPTT approximately 2.8-fold from baseline over the dose range without increasing PT. In a separate repeat-dose toxicology study in NHPs, REGN7508 also prolonged aPTT with no increase in PT or bleeding times and no detection of fecal blood.

Elevations of aPTT that are due to administration of heparin may be associated with increased bleeding risk due to the inhibition of thrombin by heparin (Alquwaizani, 2013). In contrast, prolongation of aPTT due to reductions in FXI activity is not considered a safety signal since this increase in aPTT is not necessarily associated with bleeding risk (Kitchens, 2005) (Tagariello, 2017). This is especially true in the context of genetic loss and pharmacologic inhibition of the intrinsic pathway (Koch, 2019) (Lorentz, 2019). As discussed above, FXI deficiency is generally associated with minimal spontaneous bleeding risk, although some patients experience increased bleeding after trauma or surgery to sites with high fibrinolytic activity such as the urinary tract, mouth, and nose (Asakai, 1991) (Salomon, 2006). Notably, Phase 1 studies with 3 different FXI binding mAbs have been reported. All showed ≥2-fold changes in aPTT at the highest doses, and none reported any bleeding complications (Lorentz, 2019) (Koch, 2019) (Thomas, 2019). Other agents targeting FXI activity by different mechanisms have also shown minimal bleeding risk in healthy volunteers (Thomas, 2021) (Piccini, 2022). The data generated from this study are also consistent with previous observations as no clinically significant bleeding events or AESIs have been reported to date in this ongoing study.

The length of the Inpatient Treatment Period was chosen to provide ample time to conduct dense PK/PD sampling and allow for close monitoring of the participants for AEs and for performance of the BTTs. The length of the Outpatient Clinic Follow-Up Period was designed to allow for full characterization of exposure-response relationships, to provide ample time for monitoring for AEs, and to ensure that PD effects are resolved (as judged by aPTT values) for the majority of participants receiving REGN7508 for at least a week prior to the end of follow-up (EOF). The EOF was no sooner than day 36. For lower dose-level cohorts, it was anticipated that PD effects resolve prior to day 29, and EOF would occur on day 36. For higher dose-level cohorts, it was anticipated PD effects would be observed beyond day 36, and the EOF would be extended to ensure participants are followed until PD effects resolve. The Outpatient Clinic Follow-Up Period was extended for the entire cohort and not just individuals in order to avoid inadvertent unblinding of study site personnel that might occur if individuals were scheduled for extended follow-up. However, as an additional safety measure, prolonged follow-up could be advised.

### Objectives

The primary objective of this study was to evaluate the safety and tolerability of single doses of REGN7508 in healthy adult participants. The secondary objectives include evaluation of the effects of single doses of REGN7508 on intrinsic/common pathway coagulation; evaluation of the effects of single doses of REGN7508 on extrinsic/common pathway coagulation; characterization of the drug (total and functional) concentration profiles and pharmacokinetics (PK) following single escalating doses of REGN7508; characterization of concentration profiles of total and free FXI following single escalating doses of REGN7508; and assessment of the immunogenicity of single doses of REGN7508.

The exploratory objectives of this study were to explore the effects of single doses of REGN7508 on FXI activity; explore the effects of single doses of REGN7508 on intrinsic-pathway-triggered thrombin generation; explore the effects of single doses of REGN7508 on extrinsic-pathway-triggered thrombin generation; explore the effects of single doses of REGN7508 on subclinical gastrointestinal blood loss; explore the effects of single doses of REGN7508 on bleeding time; explore the relationship between indicators of coagulation activity and systemic concentrations of REGN7508; explore the mechanism of action of REGN7508 (related to efficacy and/or safety), the coagulation cascade, and related diseases; and to explore whether potential differences in pharmacodynamics (PD) and safety are associated with genotype and gene expression and to further study FXI and coagulation-related diseases, using whole blood DNA and RNA collected from consented optional substudy participants.

### Study Design

This study was a randomized, double-blind, placebo-controlled, single-site first-in-human (FIH) study of REGN7508 (FIG. 1). The objective of this study was to evaluate the safety, tolerability, PK, and PD of REGN7508, a monoclonal antibody (mAb) that binds FXI/FXIa and prevents activation of Factor IX (FIX) by Factor XIa (FXIa), in healthy adults. This study used a single ascending dose design. Participants were randomized in a 3:1 ratio of REGN7508:placebo in up to 10 dose cohorts: 5 intravenous (IV) dose administration, 3 subcutaneous (SC) dose administration, and 2 optional cohorts.

Specifically, participants who met the eligibility criteria were randomized to 1 of the following study cohorts: IV Cohorts: Cohort 1: 5 mg IV REGN7508 or placebo, single dose; Cohort 2: ≤15 mg IV REGN7508 or placebo, single dose; Cohort 3: ≤30 mg IV REGN7508 or placebo, single dose; Cohort 4: ≤60 mg IV REGN7508 or placebo, single dose; Cohort 5: ≤125 mg IV REGN7508 or placebo, single dose. SC Cohorts: Cohort 6: ≤125 mg SC REGN7508 or placebo, single dose; Cohort 7: 250 mg SC REGN7508 or placebo, single dose; Cohort 9: ≤450 mg SC REGN7508 or placebo, single dose. Optional Cohorts: Cohort 8 (Optional Cohort): ≤250 mg IV or SC, REGN7508 or placebo, single dose; Cohort 10 (Optional Cohort): ≤600 mg SC REGN7508 or placebo, single dose.

In order to optimize safety, the first 2 participants in each cohort (sentinel participants) were dosed at least 48 hours prior to the remaining participants in the cohort and were randomized in a 1:1 manner (REGN7508:placebo). Maximal PD effects with IV doses were expected within 48 hours based on results of preclinical PK/PD studies. The remaining participants in the cohort were dosed only after both sentinel participants in the cohort have completed at least 48 hours of safety assessments and the data have been reviewed.

Escalation to the next dose cohort occurred once all participants enrolled in a cohort were observed for at least 7 days poststudy drug administration and have completed day 8 (visit 6) safety assessments and after the data have been reviewed. Any escalation in dose level did not occur until sufficient data (e.g., simulated dose predictions and empiric activated partial thromboplastin time [aPTT] data) were available to predict that the next dose level was unlikely to result in a population mean aPTT that is approximately >2× the upper limit of normal (ULN) at 2 months postdose.

The study consisted of a screening period lasting approximately 28 days, a 3-day in-patient treatment period, with administration of a single dose of REGN7508 (IV or SC) or placebo on day 1, an Outpatient Clinical Follow-up Period of approximately 33 days (minimum of 33 days; extended as needed to allow for resolution of PD effects [i.e., aPTT] at least 1 week before EOF), and a day 100 EOS telephone visit. The study flow is outlined in FIG. 1.

### Dose Selection

Single IV doses beginning with 5 mg and ≤15, ≤30, ≤60, and ≤125, as well as single SC doses≤125, ≤250 mg and ≤450 mg were selected for study. In addition there were 2 optional cohorts of ≤250 mg IV or SC as well as ≤600 mg SC (FIG. 2). Dose escalation occurred provided adequate safety/tolerability data without an untoward duration of PD effect is observed at the previous lower doses. The maximum dose used in this study did not exceed 250 mg IV or 600 mg SC, assuming adequate safety/tolerability data was observed at lower doses.

Dose selection, including the maximum dose, was informed from the totality of nonclinical pharmacology, safety, PK, and PK/PD studies, as well as the available safety, PK, and PK/PD data from lower dose cohorts in this study. Pharmacodynamic response was assessed as the fold change from baseline in aPTT values, as preclinical data demonstrate REGN7508 elevates aPTT without any measurable impact on other coagulation parameters such as PT and bleeding time. Additionally, available clinical data suggest antithrombotic effects of FXI inhibition occur with maximal prolongation of aPTT in a safe and well-tolerated manner (Lorentz, 2019). Preclinical studies were conducted in non-human primates (NHPs) because NHPs were identified as a clinically relevant species based upon similarities in the FXI amino acid sequence and plasma concentration. Furthermore, REGN7508 binding affinity and relative in vitro potency (EC₅₀) in NHPs is similar to that in humans.

Systemic concentration of REGN7508 and aPTT data from 2 NHP PK/PD studies, together with additional REGN7508 concentration data from a Good Laboratory Practice (GLP) NHP toxicology study, were used to build PK/PD models of REGN7508 in NHPs, which described the concentration of REGN7508 in serum and its relationship with changes in aPTT using a standard Hill equation (Wagner, 1968). Human PK profiles were predicted from the observed PK profile in NHPs via a standard allometric scaling approach (Dong, 2011).

Results from NHP and human plasma in vitro assays indicated comparable potency as evidenced by comparable EC₅₀ values. As these aPTT assays suggested lower EC₅₀ (2.4 and 2.7 mg/L in NHP and human plasma, respectively) compared to in vivo NHP data (8.2 mg/L), PK/PD models were constructed under 2 different assumptions:
(i) the concentration-response relationship in normal healthy volunteers (NHVs) is the same as that observed in NHP PK/PD studies, or
(ii) the concentration-response relationship in NHVs is the same as that observed in the in vitro human plasma aPTT assay.

Models incorporating these assumptions were used to compute a range of expected aPTT response profiles in NHVs. The maximum-fold change from baseline in aPTT (Eₘₐₓ) values was predicted to be 2.8-fold under assumption (i) and 3.8-fold under assumption (ii). Similarly, the concentration of REGN7508 needed to achieve 50% of the maximum effect was estimated to be 8.2 mg/L under assumption (i) and 2.7 mg/L under assumption (ii). Using simulations from the modeling described above, an IV dose of 5 mg in NHVs is predicted to yield a maximum aPTT fold change of 0% of Eₘₐₓ under assumption (i), and a maximum aPTT fold change of 6.4% of Eₘₐₓ under assumption (ii). The aPTT effect at this dose is anticipated to result in no detectable effect under assumption (i) and immediately decline from its maximum value and return to baseline within a few days under assumption (ii) and therefore is considered to represent a minimal PD effect in human participants.

Based on the above assessment, a starting dose of 5 mg REGN7508 administered as a single IV infusion was selected for this FIH clinical study, and dose escalation was planned at no more than half-log intervals (approximately 3-fold) up to a maximum of 250 mg. While simulations from the PK/PD models suggested that a single IV dose of 250 mg would achieve near-maximal aPTT prolongation and was anticipated to maintain aPTT elevations>10% above baseline values for approximately 8-13 weeks, under assumption (i) or approximately 13 weeks under assumption (ii) observed aPTT data from the 250 mg IV dose cohort show a sustained maximum prolongation for only 28 days, with a return to an average within 20% of baseline by 5 weeks postdose. The data showed that REGN7508 was well tolerated without any related serious adverse reactions observed at any dose level. In view of this overprediction of the duration of the PD response, the favorable safety profile, and the need to establish dosing regimens that achieve sustained maximal inhibition of Factor XI, higher doses were added to this FIH study. An additional single SC dose of ≤450 mg was planned, with the option to investigate a higher dose not exceeding 600 mg SC. The higher dose was only to be tested if it was projected not to produce aPTT prolongation>2-fold ULN for longer than 2 months. The SC route was selected for these 2 higher doses as it is the route most likely to be used in future clinical studies in participants.

The doses to be tested in the FIH study were supported by the results of the GLP repeat-dose, 13 week recovery toxicology study in NHP. REGN7508 was well-tolerated with a no-observed-adverse effect level (NOAEL) of 100 mg/kg IV. The exposure observed at the NOAEL in the NHPs was over 3761-fold (based on Cₘₐₓ) and approximately 14,000-fold (based on area under the curve [AUC]) greater than those predicted in humans for the starting dose of 5 mg IV. Using the predicted human PK, the calculated exposure at the maximum planned dose of 600 mg SC are a Cₘₐₓ of 73.1 mg/L and an AUC_{inf} of 3090 day mg/L. The NOAEL exposure levels are therefore over 80-fold greater (either based on Cₘₐₓ or AUC) than predicted in humans. Because the potency for REGN7508 against monkey and human Factor XI is similar, no potency adjustment is required. As a conservative margin based on single-dose exposure in the NHP (Cₘₐₓ of 2570 mg/L and AUCtau at steady state of 30,600 day mg/L) and the nominal top IV dose of 250 mg, the margins were 32 and 14, respectively and for 600 mg SC the margins were 35 and 9.9 respectively.

### Study Duration

The minimum study duration clinical follow-up (the last in-person visit) for each participant was approximately 36 days, including the 3 day Inpatient Treatment Period and 33 day Outpatient Clinic Follow-Up Period, followed by a day 100 telephone call for all participants which was the end of study. If the length of the Outpatient Clinic Follow-up was more than 100 days, then the questions from the telephone call (regarding pregnancy and SAEs) could be answered in-person at the last Outpatient Clinic Follow-up visit.

If 3 or more participants in a given cohort had aPTT values that were >10% above the ULN on the day 29 visit, then the Outpatient Clinic Follow-Up Period was extended, and all participants in the cohort continued to return to the study site every 7±2 days for follow-up visits. The Extended Follow-Up continued until fewer than 3 of the participants in the cohort have aPTT values that are >10% above the ULN. Once this occurred, all participants in the cohort returned 7±2 days later for the End of Outpatient Clinic Follow-up (EOF) visit. At the higher doses, total follow-up could be 9 weeks or longer. At the discretion of the investigator and the Sponsor, individual participant follow-up could be further extended based on the need to monitor aPTT or extend the safety evaluation.

Each participant had a day 100 telephone call to ensure compliance with the contraception rules as well as to record any new SAEs

### End of Study

The end of study was defined as the date the last participant completes the end of last in-person or telephone visit, withdraws from the study, or is lost to follow-up (i.e., the study participant can no longer be contacted by the investigator).

### Population

Adult participants were randomized in up to 5 sequential, ascending single IV dose cohorts and 3 sequential, ascending single SC dose cohorts. An additional 2 optional cohorts could be exercised at the discretion of the Sponsor. 82 participants were randomized.

The study population consisted of healthy males and females (postmenopausal or permanently unable to menstruate due to a surgical procedure or other medical condition), 18 to 55 years of age (inclusive), with low risk for bleeding or thrombosis based on medical history and physical and laboratory evaluations.

### Inclusion Criteria

A participant must have met the following criteria to be eligible for inclusion in the study:
Male or female 18 to 55 years (inclusive) at the screening visit;
Body mass index between 18.0 and 32.5 kg/m² (inclusive) at the screening visit;
Judged by the investigator to be in good health based on medical history, physical examination, vital sign measurements, and ECGs performed at screening and/or prior to administration of initial dose of study drug;
Participant is in good health based on laboratory safety testing obtained at the screening visit and/or prior to administration of initial dose of study drug; Participant with suspected or confirmed Gilbert's disease can be enrolled in the study;
Normal aPTT, normal PT, and normal platelet counts at screening period and at the day −1 visit as defined by the local laboratory;
Hemoglobin value≥11.0 g/dL for females and ≥12.9 g/dL for males at the screening and day 1 visits;
Negative FOBT during screening period;
Normal BTT at day −1 as defined by the study site;
Participant is willing and able to comply with clinic visits and study-related procedures; and
Provide informed consent signed by study participant.

### Exclusion Criteria

A participant who met any of the following criteria was excluded from the study:
History of any major surgical procedure or clinically significant physical trauma, in the opinion of the investigator, that may pose a risk to the participant by study participation;
Whole blood donation within the previous 56 days or plasma donation within the previous 7 days prior to the screening visit;
Members of the clinical site study team and/or his/her immediate family, unless prior approval granted by the Sponsor;
Pregnant or breastfeeding women;
Women of childbearing potential (WOCBP), defined as women who are fertile, following menarche until becoming postmenopausal, unless permanently surgically sterile. The only allowed permanent sterilization methods for this study are hysterectomy and/or bilateral oophorectomy;
A postmenopausal state is defined as no menses for 12 months without an alternative medical cause. A high follicle-stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to the Clinical Trial Facilitation Group guidance;
Sexually active men with WOCBP partners who are unwilling to use the following forms of medically acceptable birth control during the study through end of study (at least 100 days post-dose) or until completion of the Outpatient Clinic Follow-up (whichever is longer): vasectomy with medical assessment of surgical success OR consistent use of a condom;
History of clinically significant cardiovascular, respiratory, hepatic, renal, gastrointestinal, endocrine, hematological, psychiatric, neurological, or dermatologic disease, as assessed by the investigator, that may confound the results of the study or poses an additional risk to the participant by study participation. This includes but is not limited to history of thrombotic events, known bleeding disorders, iron-deficiency anemia, central nervous system bleeding, and gastrointestinal bleeding including that caused by hemorrhoids or anal fissures;
Presents any concern to the study investigator that might confound the results of the study or poses an additional risk to the participant by their participation in the study;
Hospitalized for any reason within 30 days of the screening visit;
Estimated glomerular filtration rate (eGFR) (using the Modification of Diet in Renal Disease study equation, the Chronic Kidney Disease Epidemiology Collaboration equation, or equivalent equation) of <60 mL/min/1.73 m² at screening;
Note: If participant has a GFR below 60 mL/min/1.73 m², one repeat test is allowed. Participant may be enrolled if repeat test demonstrates GFR of >60 mL/min/1.73 m²;
Current smoker or former smoker, including e-cigarettes, who stopped smoking within 12 months prior to the screening visit;
Confirmed positive drug test result at the screening visit and/or prior to randomization or a history of drug abuse within a year prior to the screening visit;
History of alcohol abuse within the last 2 years prior to the day 1 visit;
Is positive for human immunodeficiency virus (HIV) or hepatitis B surface antigen (HBsAg) at the screening visit. Evidence of prior hepatitis B immunization or prior resolved hepatitis B infection is not an exclusion;
Is positive for hepatitis C antibody and if so, positive for qualitative (i.e., detected or not detected) hepatitis C virus RNA test at the screening visit;
Any malignancy, except for nonmelanoma skin cancer or cervical/anus in situ, that have been resected with no evidence of metastatic disease for 3 years prior to the screening visit
History of significant multiple and/or severe allergies (e.g., latex gloves) or has had an anaphylactic reaction to prescription or nonprescription drugs or food;
Participated in any clinical research study evaluating another investigational drug including biologics or therapy, including specific immunotherapy, within 90 days or at least 5 half-lives (whichever is longer) of an investigational biologic drug or at least 4 weeks for other investigational drug prior to the screening visit;
Use of any prescription and nonprescription medications or nutritional supplements from approximately 2 weeks or 5 half-lives, whichever is longer, prior to first administration of the study drug through the EOF except the permitted medications listed. This includes anticoagulants and antiplatelets, including NSAIDs or salicylic acid; or
Unwilling or unable to comply with the permitted and prohibited medications specifications for this study.

### Treatments

REGN7508 drug product was supplied as a 265 mg lyophilized powder in sterile single-use vials. Placebo to match REGN7508 was supplied as a lyophilized powder in single sterile-use vials.

Participants were randomized in a 3:1 ratio of REGN7508:placebo in up to 10 dose cohorts: 5 IV dose administration cohorts; 3 SC dose administration cohorts; 2 optional cohorts.

Intravenous dose levels started at 5 mg. Doses for subsequent cohorts increased at no more than half-log increments. The SC dose level started at up to 100 or 125 mg and increased up to 250 or 600 mg. The maximum nominal dose in this study that could be used in an optional cohort was 250 mg IV or 600 mg SC, assuming adequate safety/tolerability data was observed at lower doses (Table 2).

TABLE 2
Study Drug Administered

| Arm Name | Investigational Arm | Placebo Arm |
|---|---|---|
| Intervention Name | REGN7508 | Placebo |
| Type | Biologic | Placebo comparator |
| Dose Formulation | Vial (lyophilized for | Vial (lyophilized for |
| | reconstitution) | reconstitution) |
| Unit Dose Strength(s) | 265 mg single use vial | 0 mg single use vial |
| Dosage Level(s) | Single IV Dose: 5 mg, 15 mg, 30 | Single IV or SC dose |
| | mg, 60 mg, 125 mg, 250 mg. | |
| | Single SC Dose: 125 mg, 250 mg, | |
| | 450 mg, 600 mg. | |
| Route of | IV or SC | IV or SC |
| Administration | | |
| Use | Experimental | Placebo comparator |
| IMP or NIMP | IMP | IMP |
| Sourcing | Provided centrally by the sponsor. | Provided centrally by the sponsor. |
| Packaging and | Study drug was provided in a vial | Study drug was provided in a vial |
| Labeling | in carton. Each vial in carton was | in carton. Each vial in carton was |
| | labeled as required per country | labeled as required per country |
| | requirement. | requirement. |

Abbreviations: IMP = investigational medicinal product; IV = intravenous; NIMP = non-investigational medicinal product; SC = subcutaneous.

### Endpoints

The primary endpoint was the incidences and seventies of treatment-emergent adverse events (TEAE) in participants treated with REGN7508 or placebo. The secondary endpoints were: change from basement in aPTT through EOF visit; change in baseline in prothrombin time (PT) through the EOF visit; concentrations of total REGN7508 in serum and functional REGN7508 in plasma through the EOF visit; absolute concentration and change from baseline in total and free FXI concentrations through the EOF visit; and incidence of antidrug antibodies to REGN7508 over time.

### Procedures and Assessments

No formal efficacy assessments were performed. Safety assessments included monitoring vital signs (semi-recumbent blood pressure, pulse, temperature, and respiration), physical examination, ECG, fecal occult blood test (FOBT), bleeding time test (BTT), laboratory evaluations (PT, hematology, chemistry, and urinalysis), and the monitoring and reporting of adverse events (AEs). Pharmacodynamics assessments were made using measurements of aPTT. Samples were collected for drug concentration, ADA incidences, concentration of total target, and exploratory assessments.

Blood samples were optionally collected and/or stored for future research and/or pharmacogenomics substudies for participants who chose to participate in the optional components of the main study.

### Activated Partial Thromboplastin Time

Activated partial thromboplastin time is a standard clinically validated measure of intrinsic/common pathway activation. Elevations of aPTT in the context of disruption to the intrinsic coagulation pathway were anticipated and are not considered a safety signal since elevations are not necessarily associated with bleeding risk. Samples for aPTT measurements were collected. Change from baseline in aPTT is a secondary endpoint and was also considered the main PD variable.

### Factor XI Functional Activity Levels

Factor XI functional activity is a clinically validated assessment of intrinsic pathway coagulation utilizing a one-stage clotting assay, but unlike aPTT, it is a more specific measure of FXI activity and its contribution to coagulation. Factor XI functional activity was measured from samples collected at time points. Modulation of FXI:C is an exploratory measure.

### Thrombin Generation Assay

Thrombin generation assay (TGA) is an in vitro and ex vivo assessment of coagulation through both the intrinsic and extrinsic pathways and provides an estimation of the ability to generate thrombin. Thrombin generation assay is not routinely used to monitor anticoagulation clinically but complements other clinically validated coagulation assessments and is useful to determine the risk of bleeding or thrombosis. Thrombin generation is an essential process of coagulation because thrombin is responsible for the activation of other coagulation factors and propagation of additional thrombin (via FXI activation) for the conversion of fibrinogen to fibrin. Using TF or ellagic acid (EA) as the trigger in the TGA can provide an estimation of the thrombin being generated by the extrinsic/common pathway and the intrinsic/common pathway, respectively. Thrombin generation was measured from samples collected at time points. Change from baseline in thrombin generation was an exploratory endpoint.

### Statistical Plan

Sample Size: 82 male and female adult participants were randomized in up to 5 sequential, ascending single IV dose cohorts and in up to 3 sequential, ascending single SC dose cohorts with 2 optional cohorts. The study was not powered to test any formal statistical hypotheses.

Based on the standard deviation (SD) of aPTT from previous studies, half-width of the 90% confidence interval for this study is well within the range to detect a clinically significant increase in aPTT values in participants receiving REGN7508.

Safety Analysis: Safety and tolerability were assessed by clinical and statistical reviews of all relevant parameters, including FOBTs, BTTs, vital signs (e.g., semi-recumbent blood pressure, pulse, temperature, and respiration), physical examinations, ECGs, laboratory evaluations (PT, hematology, chemistry, and urinalysis), and AEs.

Efficacy Analysis: The secondary objectives of the study include evaluating the effects of single doses of REGN7508 on intrinsic/common pathway coagulation and the effects of single doses of REGN7508 on extrinsic/common pathway coagulation. The PD analysis set was used for the efficacy analyses in the study. Descriptive summaries were provided for the aPTT analysis. Plots of the values over time as well as the change or percent change over time were provided.

For continuous variables, descriptive statistics were summarized. For categorical or ordinal data, frequencies and percentages were displayed for each category.

Change from baseline in aPTT values over time was summarized with descriptive statistics by dose cohorts. Pharmacodynamic summary parameters such as maximum response achievable from an applied or dosed agent (Eₘₐₓ [maximum effect]) and area under the effect vs time curve (AUEC) could be derived. In addition, dose response in the IV administration cohorts for change from baseline in aPTT was evaluated quantitatively with dose and exposure modelling, where possible. Nominal p-values were provided.

For biomarkers including Factor XI functional activity (FXI:C) and thrombin generation (following intrinsic and extrinsic pathway activation), the following descriptive data were generated: raw data at baseline, by treatment group, and overall. Biomarkers measured post-treatment will be summarized over time, and change and/or percent change from baseline to each scheduled assessment time was summarized by treatment with descriptive statistics. In addition, for each visit the mean concentrations and mean percent change from baseline was generated.

### Methods

In this first-in-human, Phase 1, randomized, double-blind, placebo-controlled, single-ascending-dose study, REGN7508 safety, tolerability, pharmacodynamics, and pharmacokinetics were evaluated in healthy volunteers (NCT05603195). Participants were randomized 3:1 to single-dose REGN7508 or placebo: REGN7508 intravenous (IV) 5-250 mg (6 cohorts); REGN7508 subcutaneous (SC)≤125-600 mg (4 cohorts). The primary endpoint was treatment-emergent adverse event (TEAE) incidence and severity. Secondary endpoints included activated partial thromboplastin time (aPTT) and prothrombin time (PT) versus baseline.

### Results

Overall, 80 participants were enrolled; baseline characteristics were balanced between cohorts. TEAEs were reported by 60% (36/60) of participants receiving REGN7508 vs 65% (13/20) receiving placebo, and treatment-related TEAEs were reported by 18% vs 20%, respectively. The most common TEAEs (≥5% of participants) in REGN7508 cohorts were headache, back pain, cough, and rhinitis. No bleeding-related adverse events, serious TEAEs, or TEAEs leading to discontinuation were reported with REGN7508. REGN7508 resulted in a rapid, prolonged, and dose-dependent extension of aPTT. An approximately 3-fold increase in aPTT from baseline was observed 30 minutes after administration of REGN7508 IV at doses≥30 mg and 1-2 days after REGN7508 SC at doses≥125 mg. Effect duration was prolonged and maintained for ≥21 days at IV or SC doses≥250 mg. REGN7508 had no effect on PT at any doses tested.

### Conclusion

Overall, REGN7508 administered IV or SC was generally well tolerated with no deaths, treatment-emergent serious adverse events (SAEs), treatment-emergent adverse events of special interest (AESIs), or treatment-emergent adverse events (TEAEs) leading to study discontinuation (Table 3), and exhibited dose-dependent inhibition of intrinsic-mediated coagulation, with durable prolongation of aPTT in this healthy volunteer first-in-human study. The incidence of TEAEs was similar between the total placebo group (65.0% of participants) and the total REGN7508 group (60.0% of participants) (Table 3). The majority of TEAEs were of mild or moderate severity. The proportion of participants that experienced treatment-related TEAEs was similar between both groups: 20.0% of participants (4/20) in the total placebo group and 18.3% of participants (11/60) in the total REGN7508 group. Notably, there was no evidence of a relationship between dose and incidence of TEAEs in the SC or IV cohorts (Tables 4 and 5). Minor differences, when observed, were likely driven by the small sample size. Overall, the mean changes in clinical laboratory values and vital signs were similar across the study groups.

An overview of the TEAEs experienced by the SAF is presented in Table 3. The incidence of TEAEs was similar between the total placebo group and the total REGN7508 group, with 65.0% of participants (13/20) in the total placebo group and 60.0% of participants (36/60) in the total REGN7508 group experiencing at least 1 TEAE, respectively (Table 3). The majority of participants experienced TEAEs that were mild or moderate in severity. There were 2 severe TEAEs reported; these occurred in participants in the total placebo group: Syncope (placebo SC total group) and Road traffic accident (placebo IV total group).

TABLE 3
Overview of Treatment-Emergent Adverse Events - All Cohorts - Safety Analysis Set

| | Placebo Total | | | REGN7508 Total | | |
|---|---|---|---|---|---|---|
| | IV | SC | IV + SC | IV | SC | IV + SC |
| Participants with at least one | (N = 12) | (N = 8) | (N = 20) | (N = 36) | (N = 24) | (N = 60) |
| TEAE | 8 (66.7%) | 5 (62.5%) | 13 (65.0%) | 24 (66.7%) | 12 (50.0%) | 36 (60.0%) |
| TEAE with Severity of Severe | 1 (8.3%) | 1 (12.5%) | 2 (10.0%) | 0 | 0 | 0 |
| TE-SAE | 0 | 0 | 0 | 0 | 0 | 0 |
| Treatment-related TEAE | 1 (8.3%) | 3 (37.5%) | 4 (20.0%) | 7 (19.4%) | 4 (16.7%) | 11 (18.3%) |
| TEAE leading to study | 0 | 0 | 0 | 0 | 0 | 0 |
| discontinuation | | | | | | |
| TEAE leading to death | 0 | 0 | 0 | 0 | 0 | 0 |
| TEAE leading to dose | 0 | 0 | 0 | 1 (2.8%) | 0 | 1 (1.7%) |
| interruption | | | | | | |

Abbreviations: IV, intravenous; N, number of participants; SAE, serious adverse event; SC, subcutaneous; TEAE, treatment-emergent adverse event.

The majority of participants (66.7%) in both the placebo IV total group and the REGN7508 IV total group experienced at least 1 TEAE (Table 4). The majority of TEAEs were mild or moderate in severity; 1 severe TEAE was reported in the placebo IV total group (Road traffic accident). The severe TEAE of Road traffic accident was reported as non-serious and assessed as not related to study drug; no bleeding events were reported for this participant.

TABLE 4
Overview of Treatment-Emergent Adverse Events - IV Cohorts - Safety Analysis Set

| | Placebo - | REGN7508 - | REGN7508 - | REGN7508 - | REGN7508 - | REGN7508 - | REGN7508 - | REGN7508 - |
|---|---|---|---|---|---|---|---|---|
| | IV | IV | IV | IV | IV | IV | IV | IV |
| Participants with at | Total | 5 mg | 15 mg | 30 mg | 60 mg | 125 mg | 250 mg | Total |
| least one | (N = 12) | (N = 6) | (N = 6) | (N = 6) | (N = 6) | (N = 6) | (N = 6) | (N = 36) |
| TEAE | 8 (66.7%) | 4 (66.7%) | 4 (66.7%) | 5 (83.3%) | 5 (83.3%) | 4 (66.7%) | 2 (33.3%) | 24 (66.7%) |
| TEAE with Severity | 1 (8.3%) | 0 | 0 | 0 | 0 | 0 | 0 | 0 |
| of Severe | | | | | | | | |
| TE-SAE | 0 | 0 | 0 | 0 | 0 | 0 | 0 | 0 |
| Treatment-related | 1 (8.3%) | 1 (16.7%) | 1 (16.7%) | 1 (16.7%) | 1 (16.7%) | 3 (50.0%) | 0 | 7 (19.4%) |
| TEAE | | | | | | | | |
| TEAE leading to | 0 | 0 | 0 | 0 | 0 | 0 | 0 | 0 |
| study discontinuation | | | | | | | | |
| TEAE leading to | 0 | 0 | 0 | 0 | 0 | 0 | 0 | 0 |
| death | | | | | | | | |
| TEAE leading to | 0 | 1 (16.7%) | 0 | 0 | 0 | 0 | 0 | 1 (2.8%) |
| dose interruption | | | | | | | | |

Abbreviations: IV, intravenous; N, number of participants; SAE, serious adverse event; TEAE, treatment-emergent adverse event.

62.5% (5/8) of participants in the placebo SC total group compared to 50.0% (12/24) in the REGN7508 SC total group reported at least 1 TEAE (Table 5). The majority of TEAEs were mild or moderate in severity; 1 severe TEAE was reported in the placebo SC total group (Syncope). The severe TEAE of Syncope was reported as non-serious and assessed as related to study drug.

TABLE 5
Overview of Treatment-Emergent Adverse Events - SC Cohorts - Safety Analysis Set

| | | REGN7508 - | REGN7508 - | REGN7508 - | REGN7508 - | REGN7508 - |
|---|---|---|---|---|---|---|
| | Placebo - SC | SC | SC | SC | SC | SC |
| | Total | 125 mg | 250 mg | 450 mg | 600 mg | Total (N = 24) |
| Participants with at least one | (N = 8) | (N = 6) | (N = 6) | (N = 6) | (N = 6) | REGN7508 - |
| TEAE | 5 (62.5%) | 2 (33.3%) | 3 (50.0%) | 3 (50.0%) | 4 (66.7%) | 12 (50.0%) |
| TEAE with Severity of | 1 (12.5%) | 0 | 0 | 0 | 0 | 0 |
| Severe | | | | | | |
| TE-SAE | 0 | 0 | 0 | 0 | 0 | 0 |
| Treatment-related TEAE | 3 (37.5%) | 0 | 1 (16.7%) | 2 (33.3%) | 1 (16.7%) | 4 (16.7%) |
| TEAE leading to study | 0 | 0 | 0 | 0 | 0 | 0 |
| discontinuation | | | | | | |
| TEAE leading to death | 0 | 0 | 0 | 0 | 0 | 0 |
| TEAE leading to dose | 0 | 0 | 0 | 0 | 0 | 0 |
| interruption | | | | | | |

Abbreviations: N, number of participants; SAE, serious adverse event; SC, subcutaneous; TEAE, treatment-emergent adverse event.

An overview of the frequency of TEAEs by SOC and PT is presented in Table 6. Among participants who received REGN7508, the percentage of participants who experienced at least 1 TEAE was higher with IV administration (24/36; 66.7%) compared with SC administration (12/24; 50.0%). SOCs with TEAEs observed at a ≥5% incidence in the total REGN7508 group compared to the total placebo group were: Respiratory, thoracic and mediastinal disorders (11.7% REGN7508 versus 5.0% placebo); Infections and infestations (10.0% REGN7508 versus 5.0% placebo); Skin and subcutaneous tissue disorders (5.0% REGN7508 versus 0% placebo) (Table 6).

For the SOC of Respiratory, thoracic and mediastinal disorders, the difference was driven by the incidence of Cough (5.0% REGN7508 versus 0% placebo) (Table 6). Cough was reported by 1 participant who received REGN7508 125 mg SC and 2 participants who received REGN7508 250 mg SC. For the SOC of Infections and infestations, the difference was driven by the incidence of Rhinitis (5.0% REGN7508 versus 0% placebo) (Table 6). Rhinitis was reported by 2 participants who received REGN7508 IV: 1 participant in each of the REGN7508 15 mg and 60 mg IV groups, respectively, and 1 participant who received REGN7508 SC (450 mg). For the SOC of Skin and subcutaneous tissue disorders, the difference was driven by the incidence of Miliaria (1 participant in the REGN7508 30 mg IV group), Ingrowing nail (1 participant in the REGN7508 60 mg IV group), and Rash macular (1 participant in the REGN7508 125 mg SC group) in participants who received REGN7508. No participants who received placebo reported TEAEs in the SOC of Skin and subcutaneous tissue disorders (Table 6).

TABLE 6
Treatment-Emergent Adverse Events by Primary System Organ Class
and Preferred Term - All Cohorts - Safety Analysis Set - Overall

| | Placebo Total | | | REGN7508 Total | | |
|---|---|---|---|---|---|---|
| Primary System Organ Class | IV | SC | IV + SC | IV | SC | IV + SC |
| Preferred Term | (N = 12) | (N = 8) | (N = 20) | (N = 36) | (N = 24) | (N = 60) |
| Participants with at least one such | 8 (66.7%) | 5 (62.5%) | 13 (65.0%) | 24 (66.7%) | 12 (50.0%) | 36 (60.0%) |
| TEAE, n (%) | | | | | | |
| Nervous system disorders | 1 (8.3%) | 3 (37.5%) | 4 (20.0%) | 8 (22.2%) | 4 (16.7%) | 12 (20.0%) |
| Headache | 1 (8.3%) | 1 (12.5%) | 2 (10.0%) | 5 (13.9%) | 4 (16.7%) | 9 (15.0%) |
| Dizziness | 0 | 0 | 0 | 2 (5.6%) | 0 | 2 (3.3%) |
| Disturbance in attention | 0 | 0 | 0 | 1 (2.8%) | 0 | 1 (1.7%) |
| Drug withdrawal headache | 0 | 0 | 0 | 1 (2.8%) | 0 | 1 (1.7%) |
| Presyncope | 0 | 1 (12.5%) | 1 (5.0%) | 0 | 0 | 0 |
| Syncope | 0 | 1 (12.5%) | 1 (5.0%) | 0 | 0 | 0 |
| General disorders and administration | 1 (8.3%) | 1 (12.5%) | 2 (10.0%) | 6 (16.7%) | 2 (8.3%) | 8 (13.3%) |
| site conditions | | | | | | |
| Catheter site bruise | 0 | 0 | 0 | 1 (2.8%) | 1 (4.2%) | 2 (3.3%) |
| Catheter site pain | 0 | 0 | 0 | 1 (2.8%) | 1 (4.2%) | 2 (3.3%) |
| Catheter site swelling | 0 | 0 | 0 | 2 (5.6%) | 0 | 2 (3.3%) |
| Influenza like illness | 0 | 0 | 0 | 2 (5.6%) | 0 | 2 (3.3%) |
| Chills | 1 (8.3%) | 0 | 1 (5.0%) | 0 | 1 (4.2%) | 1 (1.7%) |
| Feeling cold | 0 | 0 | 0 | 1 (2.8%) | 0 | 1 (1.7%) |
| Pyrexia | 0 | 0 | 0 | 1 (2.8%) | 0 | 1 (1.7%) |
| Injection site reaction | 0 | 1 (12.5%) | 1 (5.0%) | 0 | 0 | 0 |
| Gastrointestinal disorders | 2 (16.7%) | 2 (25.0%) | 4 (20.0%) | 3 (8.3%) | 4 (16.7%) | 7 (11.7%) |
| Abdominal pain | 0 | 1 (12.5%) | 1 (5.0%) | 0 | 1 (4.2%) | 1 (1.7%) |
| Constipation | 0 | 0 | 0 | 1 (2.8%) | 0 | 1 (1.7%) |
| Diarrhoea | 0 | 0 | 0 | 0 | 1 (4.2%) | 1 (1.7%) |
| Dyspepsia | 0 | 0 | 0 | 1 (2.8%) | 0 | 1 (1.7%) |
| Faeces discoloured | 0 | 0 | 0 | 1 (2.8%) | 0 | 1 (1.7%) |
| Haematochezia | 0 | 0 | 0 | 0 | 1 (4.2%) | 1 (1.7%) |
| Nausea | 1 (8.3%) | 0 | 1 (5.0%) | 1 (2.8%) | 0 | 1 (1.7%) |
| Vomiting | 1 (8.3%) | 1 (12.5%) | 2 (10.0%) | 0) | 1 (4.2%) | 1 (1.7%) |
| Abdominal discomfort | 0 | 1 (12.5%) | 1 (5.0%) | 0 | 0 | 0 |
| Abdominal pain lower | 1 (8.3%) | 0 | 1 (5.0%) | 0 | 0 | 0 |
| Respiratory, thoracic and mediastinal | 0 | 1 (12.5%) | 1 (5.0%) | 4 (11.1%) | 3 (12.5%) | 7 (11.7%) |
| disorders | | | | | | |
| Cough | 0 | 0 | 0 | 0 | 3 (12.5%) | 3 (5.0%) |
| Nasal congestion | 0 | 1 (12.5%) | 1 (5.0%) | 1 (2.8%) | 1 (4.2%) | 2 (3.3%) |
| Oropharyngeal pain | 0 | 0 | 0 | 1 (2.8%) | 1 (4.2%) | 2 (3.3%) |
| Epistaxis | 0 | 1 (12.5%) | 1 (5.0%) | 1 (2.8%) | 0 | 1 (1.7%) |
| Oropharyngeal discomfort | 0 | 0 | 0 | 1 (2.8%) | 0 | 1 (1.7%) |
| Infections and infestations | 1 (8.3%) | 0 | 1 (5.0%) | 4 (11.1%) | 2 (8.3%) | 6 (10.0%) |
| Rhinitis | 0 | 0 | 0 | 2 (5.6%) | 1 (4.2%) | 3 (5.0%) |
| COVID-19 | 0 | 0 | 0 | 1 (2.8%) | 1 (4.2%) | 2 (3.3%) |
| Ear infection | 0 | 0 | 0 | 1 (2.8%) | 0 | 1 (1.7%) |
| Nasopharyngitis | 1 (8.3%) | 0 | 1 (5.0%) | 0 | 0 | 0) |
| Musculoskeletal and connective | 0 | 2 (25.0%) | 2 (10.0%) | 1 (2.8%) | 4 (16.7%) | 5 (8.3%) |
| tissue disorders | | | | | | |
| Back pain | 0 | 1 (12.5%) | 1 (5.0%) | 1 (2.8%) | 3 (12.5%) | 4 (6.7%) |
| Pain in extremity | 0 | 0 | 0 | 0 | 2 (8.3%) | 2 (3.3%) |
| Myalgia | 0 | 1 (12.5%) | 1 (5.0%) | 0 | 0 | 0 |
| Injury, poisoning and procedural | 3 (25.0%) | 0 | 3 (15.0%) | 3 (8.3%) | 1 (4.2%) | 4 (6.7%) |
| complications | | | | | | |
| Arthropod sting | 0 | 0 | 0 | 1 (2.8%) | 0 | 1 (1.7%) |
| Hand fracture | 0 | 0 | 0 | 1 (2.8%) | 0 | 1 (1.7%) |
| Head injury | 0 | 0 | 0 | 0 | 1 (4.2%) | 1 (1.7%) |
| Skin abrasion | 1 (8.3%) | 0 | 1 (5.0%) | 1 (2.8%) | 0 | 1 (1.7%) |
| Eye injury | 1 (8.3%) | 0 | 1 (5.0%) | 0 | 0 | 0 |
| Road traffic accident | 1 (8.3%) | 0 | 1 (5.0%) | 0 | 0 | 0 |
| Thermal burn | 1 (8.3%) | 0 | 1 (5.0%) | 0 | 0 | 0 |
| Skin and subcutaneous tissue | 0 | 0 | 0 | 2 (5.6%) | 1 (4.2%) | 3 (5.0%) |
| disorders | | | | | | |
| Ingrowing nail | 0 | 0 | 0 | 1 (2.8%) | 0 | 1 (1.7%) |
| Miliaria | 0 | 0 | 0 | 1 (2.8%) | 0 | 1 (1.7%) |
| Rash macular | 0 | 0 | 0 | 0 | 1 (4.2%) | 1 (1.7%) |
| Eye disorders | 0 | 0 | 0 | 0 | 2 (8.3%) | 2 (3.3%) |
| Blepharitis | 0 | 0 | 0 | 0 | 1 (4.2%) | 1 (1.7%) |
| Ocular discomfort | 0 | 0 | 0 | 0 | 1 (4.2%) | 1 (1.7%) |
| Blood and lymphatic system | 0 | 0 | 0 | 1 (2.8%) | 0 | 1 (1.7%) |
| disorders | | | | | | |
| Lymphadenopathy | 0 | 0 | 0 | 1 (2.8%) | 0 | 1 (1.7%) |
| Investigations | 0 | 0 | 0 | 1 (2.8%) | 0 | 1 (1.7%) |
| Occult blood positive | 0 | 0 | 0 | 1 (2.8%) | 0 | 1 (1.7%) |
| Psychiatric disorders | 0 | 0 | 0 | 1 (2.8%) | 0 | 1 (1.7%) |
| Insomnia | 0 | 0 | 0 | 1 (2.8%) | 0 | 1 (1.7%) |
| Immune system disorders | 1 (8.3%) | 0 | 1 (5.0%) | 0 | 0 | 0 |
| Allergy to arthropod sting | 1 (8.3%) | 0 | 1 (5.0%) | 0 | 0 | 0 |

Abbreviations: IV, intravenous; N, number of participants; SC, subcutaneous; TEAE, treatment-emergent adverse event.
MedDRA (Version 27.0) coding dictionary applied.
A participant who reported 2 or more TEAEs with the same preferred term was counted only once for that term.
A participant who reported 2 or more TEAEs with different preferred terms within the same system organ class was counted only once in that system organ class.
Sorting of this table is based on decreasing frequency of the total treatment group.

The majority of participants experienced TEAEs that were mild or moderate in severity. The proportion of participants who experienced at least 1 mild or moderate TEAE was comparable between the total placebo group (mild TEAE: 40.0% [8/20]; moderate TEAE: 15.0% [3/20]) and the total REGN7508 group (mild TEAE: 43.3% [26/60]; moderate TEAE: 16.7% [10/60]). Two participants experienced at least 1 severe TEAE during the study; both participants were in the total placebo group. One participant in the placebo IV total group experienced a severe TEAE of Road traffic accident; the TEAE was reported as non-serious and assessed as not related to study drug. One participant in the placebo SC total group experienced a severe TEAE of Syncope; the TEAE was reported as non-serious and assessed as related to study drug.

The percentage of participants who experienced a treatment-related TEAE was similar between the total placebo group (20.0%; 4/20) and the total REGN7508 group (18.3%; 11/60) (Table 7). The most frequent treatment-related TEAE in participants treated with REGN7508 was Headache (8.3%; 5/60 REGN7508 versus 0% placebo). Among participants who received REGN7508, the frequency of the treatment-related TEAEs in the IV cohort (19.4%; 7/36) was comparable to the SC cohort (16.7%; 4/24). There was no dose-dependent trend in the total number of treatment-related TEAEs reported across the individual REGN7508 IV and SC groups.

TABLE 7
Treatment-Related Treatment-Emergent Adverse Events by Primary System Organ
Class and Preferred Term - All Cohorts - Safety Analysis Set - Overall

| | Placebo Total | | | REGN7508 Total | | |
|---|---|---|---|---|---|---|
| Primary System Organ Class | IV | SC | IV + SC | IV | SC | IV + SC |
| Preferred Term | (N = 12) | (N = 8) | (N = 20) | (N = 36) | (N = 24) | (N = 60) |
| Participants with at least one such | 1 (8.3%) | 3 (37.5%) | 4 (20.0%) | 7 (19.4%) | 4 (16.7%) | 11 (18.3%) |
| TEAE, n(%) | | | | | | |
| Nervous system disorders | 0 | 1 (12.5%) | 1 (5.0%) | 3 (8.3%) | 2 (8.3%) | 5 (8.3%) |
| Headache | 0 | 0 | 0 | 3 (8.3%) | 2 (8.3%) | 5 (8.3%) |
| Syncope | 0 | 1 (12.5%) | 1 (5.0%) | 0 | 0) | 0 |
| Gastrointestinal disorders | 1 (8.3%) | 1 (12.5%) | 2 (10.0%) | 2 (5.6%) | 2 (8.3%) | 4 (6.7%) |
| Constipation | 0 | 0 | 0 | 1 (2.8%) | 0 | 1 (1.7%) |
| Faeces discoloured | 0 | 0 | 0 | 1 (2.8%) | 0 | 1 (1.7%) |
| Haematochezia | 0 | 0 | 0 | 0 | 1 (4.2%) | 1 (1.7%) |
| Vomiting | 1 (8.3%) | 0 | 1 (5.0%) | 0 | 1 (4.2%) | 1 (1.7%) |
| Abdominal discomfort | 0 | 1 (12.5%) | 1 (5.0%) | 0 | 0 | 0 |
| Abdominal pain | 0 | 1 (12.5%) | 1 (5.0%) | 0 | 0 | 0 |
| Nausea | 1 (8.3%) | 0 | 1 (5.0%) | 0 | 0 | 0 |
| General disorders and administration | 0 | 1 (12.5%) | 1 (5.0%) | 1 (2.8%) | 1 (4.2%) | 2 (3.3%) |
| site conditions | | | | | | |
| Chills | 0 | 0 | 0 | 0 | 1 (4.2%) | 1 (1.7%) |
| Feeling cold | 0 | 0 | 0 | 1 (2.8%) | 0 | 1 (1.7%) |
| Injection site reaction | 0 | 1 (12.5%) | 1 (5.0%) | 0 | 0 | 0 |
| Investigations | 0 | 0 | 0 | 1 (2.8%) | 0 | 1 (1.7%) |
| Occult blood positive | 0 | 0 | 0 | 1 (2.8%) | 0 | 1 (1.7%) |
| Respiratory, thoracic and mediastinal | 0 | 1 (12.5%) | 1 (5.0%) | 1 (2.8%) | 0 | 1 (1.7%) |
| disorders | | | | | | |
| Epistaxis | 0 | 1 (12.5%) | 1 (5.0%) | 1 (2.8%) | 0 | 1 (1.7%) |

Abbreviations: IV, intravenous; SC, subcutaneous.
A participant who reported 2 or more TEAEs with the same preferred term is counted only once for that term.
A participant who reported 2 or more TEAEs with different preferred terms within the same system organ class is counted only once in that system organ class.

Sorting of this table is based on decreasing frequency of the total treatment group.

There were no TEAEs leading to death reported in this study (Table 3). There were no treatment-emergent SAEs reported in this study (Table 3). There were no treatment-emergent SAEs reported in this study (Table 3). This was a single-dose administration study. There were no dose modifications due to TEAEs. There were no TEAEs resulting in permanent study discontinuation (Table 3). Protocol-defined AESIs for this study were: Any moderate to severe bleeding (both spontaneous and non-spontaneous); Any moderate to severe infusion reactions; Any severe injection site reactions (ISRs); Any moderate or severe hypersensitivity reactions potentially related to study treatment. There were no treatment-emergent AESIs reported in this study. There were no infusion-related reactions (IRRs) reported during the study.

No participants (0/24; 0%) in the REGN7508 SC total group experienced an ISR. One participant (1/8; 12.5%) in the placebo SC total group experienced an ISR. The TEAE of ISR was nonserious, mild, and assessed as related to study drug.

### Evaluation of Clinical Laboratory Tests

Clinical laboratory safety parameters evaluated in this study were: Hematology; Blood chemistry; Urinalysis; Bleeding time test (BTT); Fecal occult blood test (FOBT). The data showed no dose-dependent trends or clinically meaningful changes in hematology, blood chemistry, urinalysis, BTT, or the incidence of positive FOBT. Identified laboratory abnormalities were isolated abnormal findings with no associated clinical manifestations deemed to be related to study drug.

There were no dose-dependent trends observed in the incidence of positive FOBT, and no apparent differences were observed across the IV and SC cohorts and the total placebo and total REGN7508 groups.

### Pharmacokinetics

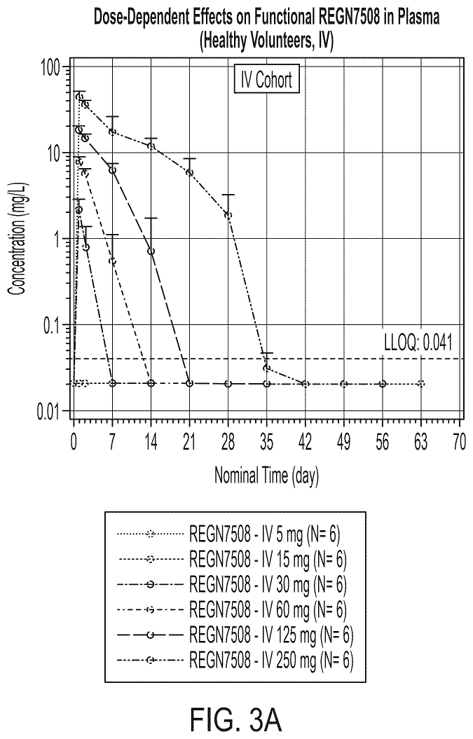

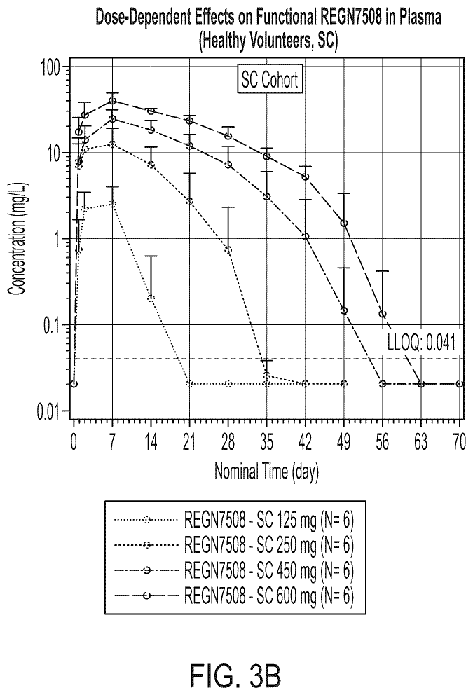

Concentrations of functional REGN7508 in plasma following a single IV dose were below the limit of quantitation (BLQ) at all timepoints in the 5 and 15 mg dose cohorts and quantifiable for only 2, 7, and 14 days following IV doses of 30 mg, 60 mg, and 125 mg, respectively. SC doses were quantifiable for functional REGN7508, 14 days post dose following the 125 mg dose. Following single doses of REGN7508, the concentration-time profiles of functional REGN7508 were characterized by a brief distribution (IV) or absorption (SC) phase, followed by a linear elimination phase and a terminal nonlinear, target-mediated elimination phase. Target-mediated disposition is evidenced by faster elimination at lower doses for both IV and SC administration (FIGS. 3A-3B). Maximum concentrations of REGN7508 were achieved immediately following IV administration and about 5-8 days following SC administration.

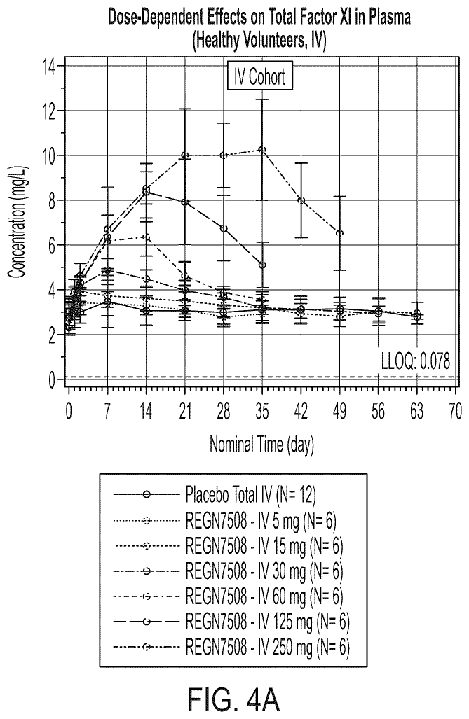

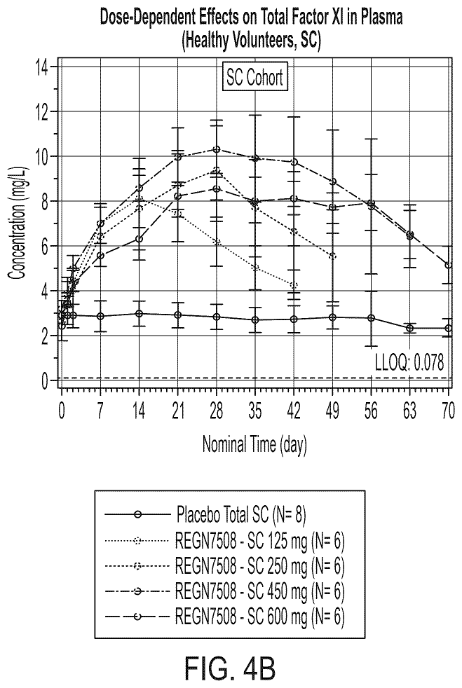

The total FXI concentration vs time profiles at each dose are presented at FIGS. 4A-4B. REGN7508 treatment resulted in a dose-dependent increase in total FXI concentrations up to a dose of 250 mg IV or 450 mg SC. An apparent decrease was observed in total FXI concentration at the 600 mg SC dose relative to 450 mg; however, the fold change from baseline was higher at 600 mg compared to 450 mg. Maximal increase in FXI concentration was approximately 3-fold and was sustained for up to 8 weeks post-dose at the top dose of 600 mg SC. Even though the plot shows a decrease in total FXI concentrations with the 600 mg SC dose, when the effect is plotted as fold change from baseline, the magnitude of the change was shown to be dose dependent throughout the range of doses tested.

The PK parameters were determined by noncompartmental methods using Phoenix® WinNonlin® Version 8.3.4 and are summarized in Table 8 by route of administration and dose.

All timepoints in the 5 and 15 mg IV dose cohorts were below the limit of quantitation (BLQ) and quantifiable for only 2, 7, and 14 days following IV doses of 30 mg, 60 mg, and 125 mg, respectively, hence area under the curve (AUC) and, where appropriate, other derived parameters were reported only for doses of 30 mg and above. Maximum concentration (Cₘₐₓ) increased more than proportionally from 30 to 60 mg and approximately proportionally to dose following 60 to 250 mg following IV dosing, while AUC values increased more than dose-proportionally (Table 8). Observed Cₘₐₓ increased more than proportionally between 125 mg and 250 mg and approximately proportionally from 250 mg to 600 mg. AUC values increase more than dose proportionally between 125 mg and 450 mg and approximately proportionally between 450 mg and 600 mg. Tₘₐₓ was approximately 5 to 8 days across dose levels (Table 8).

TABLE 8
Summary of Pharmacokinetic Parameters Calculated from Concentrations of Functional
REGN7508 in Plasma by Treatment Group in Healthy Adult Participants (PKAS)

| | (N = 6) | | | | | |
|---|---|---|---|---|---|---|
| Parameter (Units) | n | Mean | Median | SD | Min | Max |
| | REGN7508 - IV 5 mg | | | | | |
| Cₘₐₓ (mg/L) | 6 | 0 | 0 | 0 | 0 | 0 |
| Cₘₐₓ/Dose (mg/L/mg) | 6 | 0 | 0 | 0 | 0 | 0 |
| tₘₐₓ (day) | 6 | 0 | 0 | 0 | 0 | 0 |
| | REGN7508 - IV 15 mg | | | | | |
| Cₘₐₓ (mg/L) | 6 | 0 | 0 | 0 | 0 | 0 |
| Cₘₐₓ/Dose (mg/L/mg) | 6 | 0 | 0 | 0 | 0 | 0 |
| tₘₐₓ (day) | 6 | 0 | 0 | 0 | 0 | 0 |
| | REGN7508 - IV 30 mg | | | | | |
| AUCₗₐₛₜ (day*mg/L) | 6 | 1.98 | 2.02 | 0.574 | 1.03 | 2.61 |
| AUC_{last/Dose} (day*mg/L/mg) | 6 | 0.0661 | 0.0675 | 0.0191 | 0.0343 | 0.0870 |
| Cₘₐₓ (mg/L) | 6 | 2.23 | 2.11 | 0.613 | 1.61 | 3.41 |
| Cₘₐₓ/Dose (mg/L/mg) | 6 | 0.0743 | 0.0704 | 0.0204 | 0.0537 | 0.114 |
| tₘₐₓ (day) | 6 | 1.21 | 1.04 | 0.411 | 1.04 | 2.05 |
| tₗₐₛₜ (day) | 6 | 1.71 | 2.04 | 0.517 | 1.04 | 2.05 |
| | REGN7508 - IV 60 mg | | | | | |
| AUCₗₐₛₜ (day*mg/L) | 6 | 18.8 | 18.3 | 8.14 | 10.3 | 30.6 |
| AUC_{last/Dose} (day*mg/L/mg) | 6 | 0.314 | 0.305 | 0.136 | 0.171 | 0.510 |
| Cₘₐₓ (mg/L) | 6 | 7.74 | 7.68 | 1.15 | 6.06 | 9.56 |
| Cₘₐₓ/Dose (mg/L/mg) | 6 | 0.129 | 0.128 | 0.0192 | 0.101 | 0.159 |
| tₘₐₓ (day) | 6 | 1.04 | 1.04 | 0.00114 | 1.04 | 1.04 |
| tₗₐₛₜ (day) | 6 | 5.32 | 6.92 | 2.54 | 2.04 | 7.00 |
| | REGN7508 - IV 125 mg | | | | | |
| AUCₗₐₛₜ (day*mg/L) | 6 | 89.5 | 88.8 | 25.4 | 63.0 | 131 |
| AUC_{last/Dose} (day*mg/L/mg) | 6 | 0.716 | 0.711 | 0.203 | 0.504 | 1.05 |
| AUC_{inf} (day*mg/L) | 4 | 108 | 99.1 | 28.8 | 85.5 | 150 |
| AUC_{inf/Dose} (day*mg/L/mg) | 4 | 0.868 | 0.793 | 0.230 | 0.684 | 1.20 |
| _{AUCextrap} (%) | 4 | 4.59 | 2.40 | 5.36 | 1.03 | 12.6 |
| Cₘₐₓ (mg/L) | 6 | 18.1 | 18.2 | 2.42 | 15.4 | 20.9 |
| Cₘₐₓ/Dose (mg/L/mg) | 6 | 0.145 | 0.146 | 0.0193 | 0.123 | 0.167 |
| tₘₐₓ (day) | 6 | 1.04 | 1.04 | 0.00106 | 1.04 | 1.04 |
| tₗₐₛₜ (day) | 6 | 12.2 | 14.5 | 4.02 | 7.00 | 15.0 |
| t_{1/2} (day) | 4 | 3.10 | 2.65 | 1.20 | 2.24 | 4.84 |
| CL (L/day) | 4 | 1.21 | 1.26 | 0.269 | 0.832 | 1.46 |
| Vₛₛ (L) | 4 | 6.11 | 6.14 | 0.283 | 5.75 | 6.42 |
| | REGN7508 - IV 250 mg | | | | | |
| AUCₗₐₛₜ (day*mg/L) | 6 | 374 | 382 | 86.9 | 248 | 489 |
| AUC_{last/Dose} (day*mg/L/mg) | 6 | 1.50 | 1.53 | 0.348 | 0.994 | 1.95 |
| AUC_{inf} (day*mg/L) | 4 | 389 | 388 | 112 | 253 | 525 |
| AUC_{inf/Dose} (day*mg/L/mg) | 4 | 1.55 | 1.55 | 0.447 | 1.01 | 2.10 |
| AUCₑₓₜᵣₐₚ (%) | 6 | 2.00 | 1.44 | 2.59 | 0.0419 | 6.98 |
| Cₘₐₓ (mg/L) | 6 | 44.1 | 44.2 | 7.36 | 34.2 | 52.9 |
| Cₘₐₓ/Dose (mg/L/mg) | 6 | 0.177 | 0.177 | 0.0294 | 0.137 | 0.212 |
| tₘₐₓ (day) | 6 | 1.04 | 1.04 | 0.00161 | 1.04 | 1.05 |
| tₗₐₛₜ (day) | 6 | 29.0 | 28.0 | 5.06 | 21.0 | 35.0 |
| t_{1/2} (day) | 4 | 5.05 | 4.68 | 1.82 | 3.38 | 7.47 |
| CL (L/day) | 4 | 0.688 | 0.645 | 0.215 | 0.476 | 0.987 |
| Vₛₛ (L) | 4 | 5.82 | 5.58 | 0.665 | 5.36 | 6.77 |
| | REGN7508 - SC 125 mg | | | | | |
| AUCₗₐₛₜ (day*mg/L) | 5 | 19.7 | 14.6 | 12.3 | 12.3 | 41.4 |
| AUC_{last/Dose} (day*mg/L/mg) | 5 | 0.157 | 0.116 | 0.0987 | 0.0987 | 0.331 |
| Cₘₐₓ (mg/L) | 6 | 2.72 | 3.06 | 1.50 | 0 | 4.50 |
| Cₘₐₓ/Dose (mg/L/mg) | 6 | 0.0217 | 0.0245 | 0.0120 | 0 | 0.0360 |
| tₘₐₓ (day) | 6 | 5.00 | 7.00 | 3.17 | 0 | 7.01 |
| tₗₐₛₜ (day) | 5 | 8.41 | 7.01 | 3.14 | 7.00 | 14.0 |
| | REGN7508 - SC 250 mg | | | | | |
| AUCₗₐₛₜ (day*mg/L) | 6 | 177 | 132 | 133 | 86.1 | 433 |
| AUC_{last/Dose} (day*mg/L/mg) | 6 | 0.709 | 0.526 | 0.532 | 0.344 | 1.73 |
| AUC_{inf} (day*mg/L) | 2 | 149 | — | — | 99.6 | 199 |
| AUC_{inf/Dose} (day*mg/L/mg) | 2 | 0.596 | — | — | 0.398 | 0.795 |
| AUCₑₓₜᵣₐₚ (%) | 3 | 1.87 | 2.30 | 1.66 | 0.0318 | 3.27 |
| Cₘₐₓ (mg/L) | 6 | 13.3 | 10.7 | 8.39 | 6.40 | 28.4 |
| Cₘₐₓ/Dose (mg/L/mg) | 6 | 0.0530 | 0.0426 | 0.0335 | 0.0256 | 0.114 |
| tₘₐₓ (day) | 6 | 5.35 | 7.01 | 2.60 | 2.00 | 7.06 |
| tₗₐₛₜ (day) | 6 | 24.3 | 21.0 | 5.99 | 20.0 | 35.0 |
| t_{1/2} (day) | 2 | 3.79 | — | — | 3.78 | 3.80 |
| CL/F (L/day) | 2 | 1.88 | — | — | 1.26 | 2.51 |
| Vz/F (L) | 2 | 10.3 | — | — | 6.86 | 13.8 |
| | REGN7508 - SC 450 mg | | | | | |
| AUCₗₐₛₜ (day*mg/L) | 6 | 475 | 447 | 186 | 242 | 811 |
| AUC_{last/Dose} (day*mg/L/mg) | 6 | 1.05 | 0.994 | 0.414 | 0.538 | 1.80 |
| AUC_{inf} (day*mg/L) | 3 | 578 | 499 | 211 | 417 | 817 |
| AUC_{inf/Dose} (day*mg/L/mg) | 3 | 1.28 | 1.11 | 0.470 | 0.928 | 1.82 |
| AUCₑₓₜᵣₐₚ (%) | 5 | 0.830 | 0.400 | 1.22 | 0.0307 | 2.95 |
| Cₘₐₓ (mg/L) | 6 | 24.3 | 23.7 | 7.08 | 16.0 | 36.7 |
| Cₘₐₓ/Dose (mg/L/mg) | 6 | 0.0541 | 0.0526 | 0.0157 | 0.0356 | 0.0816 |
| tmax (day) | 6 | 7.01 | 7.00 | 0.00651 | 7.00 | 7.02 |
| tlast (day) | 6 | 38.5 | 38.5 | 10.6 | 21.0 | 49.0 |
| t1/2 (day) | 3 | 4.37 | 5.05 | 1.30 | 2.88 | 5.19 |
| CL/F (L/day) | 3 | 0.844 | 0.903 | 0.269 | 0.551 | 1.08 |
| Vz/F (L) | 3 | 5.06 | 4.48 | 1.33 | 4.12 | 6.58 |
| | REGN7508 - SC 600 mg | | | | | |
| AUCₗₐₛₜ (day*mg/L) | 6 | 925 | 935 | 134 | 719 | 1110 |
| AUC_{last/Dose} (day*mg/L/mg) | 6 | 1.54 | 1.56 | 0.224 | 1.20 | 1.85 |
| AUC_{inf} (day*mg/L) | 4 | 943 | 950 | 170 | 740 | 1130 |
| AUC_{inf/Dose} (day*mg/L/mg) | 4 | 1.57 | 1.58 | 0.284 | 1.23 | 1.89 |
| AUCₑₓₜᵣₐₚ (%) | 6 | 1.67 | 1.51 | 1.65 | 0.0156 | 4.12 |
| Cₘₐₓ (mg/L) | 6 | 40.8 | 40.5 | 8.07 | 27.4 | 51.4 |
| Cₘₐₓ/Dose (mg/L/mg) | 6 | 0.0680 | 0.0674 | 0.0134 | 0.0457 | 0.0857 |
| tₘₐₓ (day) | 6 | 8.17 | 7.01 | 2.86 | 6.99 | 14.0 |
| tₗₐₛₜ (day) | 6 | 49.0 | 49.0 | 4.41 | 42.0 | 56.0 |
| t_{1/2} (day) | 4 | 7.29 | 7.42 | 0.690 | 6.35 | 7.97 |
| CL/F (L/day) | 4 | 0.653 | 0.635 | 0.123 | 0.529 | 0.811 |
| Vz/F (L) | 4 | 6.91 | 7.13 | 1.62 | 4.85 | 8.52 |

n = Number of participants contributing to the statistical results; SD = Standard deviation

### Pharmacodynamics

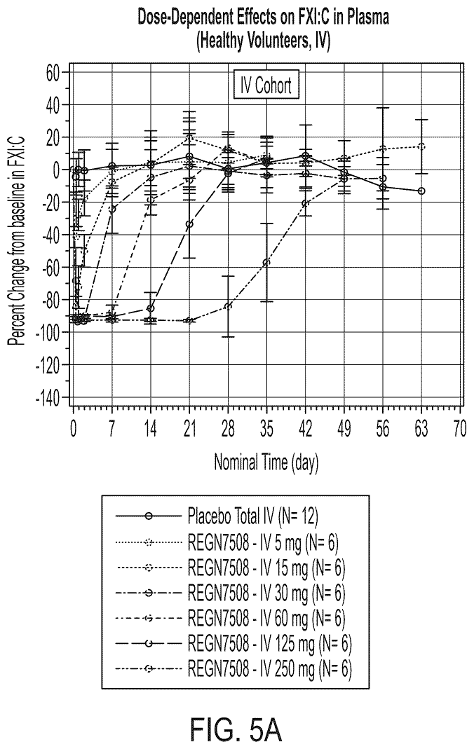

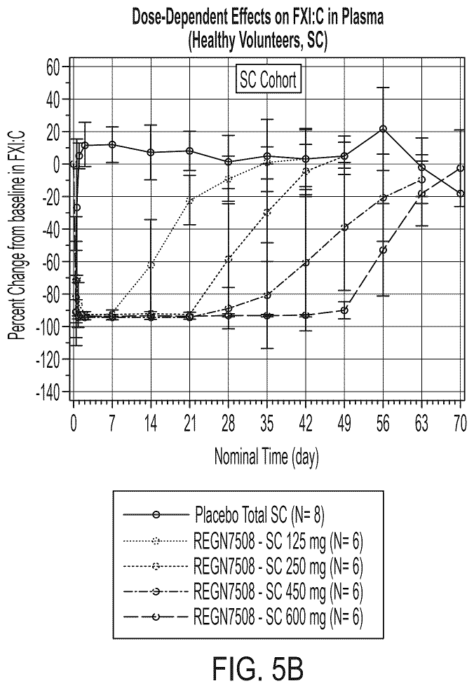

Mean percent change from baseline in FXI:C vs time profiles are presented in FIGS. 5A-5B. REGN7508 treatment produced a rapid decrease in FXI:C. The magnitude and duration of the decrease in FXI:C was REGN7508 dose-dependent. Maximal effect (≥92% inhibition) was maintained for ~21 days with doses of 250 mg (IV and SC) and 450 mg and ~42 days post-dose with the 600 mg SC dose (FIGS. 5A-5B).

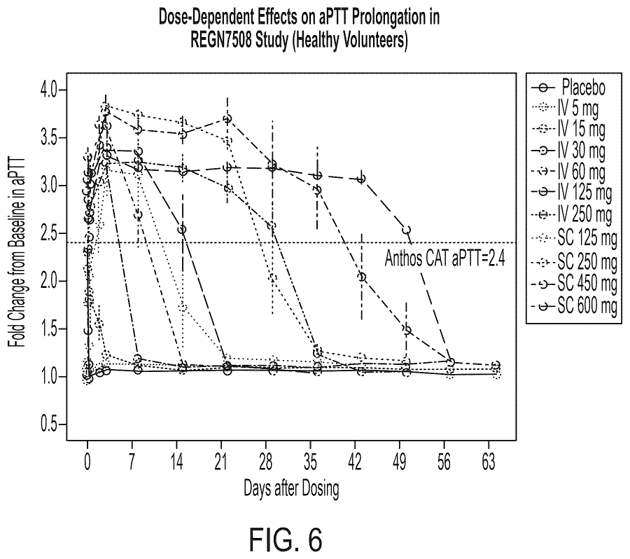

In clotting assays, REGN7508 increased aPTT relative to baseline (i.e., no antibody) in a concentration-dependent manner, see FIG. 6; increases of up to 2.5-fold in plasma were observed at the range of doses tested (REGN7508 5-250 mg IV, 125-600 mg SC). The 2-fold increase of aPTT relative to baseline was estimated to occur at ≥30 mg IV, ≥125 mg SC for REGN7508 in participant plasma. Following the single dose administration of the anti-FXI monoclonal antibody, aPTT values returned to within 10% of normal by Day 64. No change in PT relative to baseline was observed up to the maximum antibody concentration tested.

A dose-dependent prolongation of the aPTT was observed with a mean peak of approximately 3.3-fold elevation in aPTT observed at the 250 mg IV dose level, which was maintained for >21 days for all participants and decreased to mean aPTT elevation of 1.2-fold by day 36. No dose-dependent trends have been observed in the PT, BTT or FOBT tests to date.

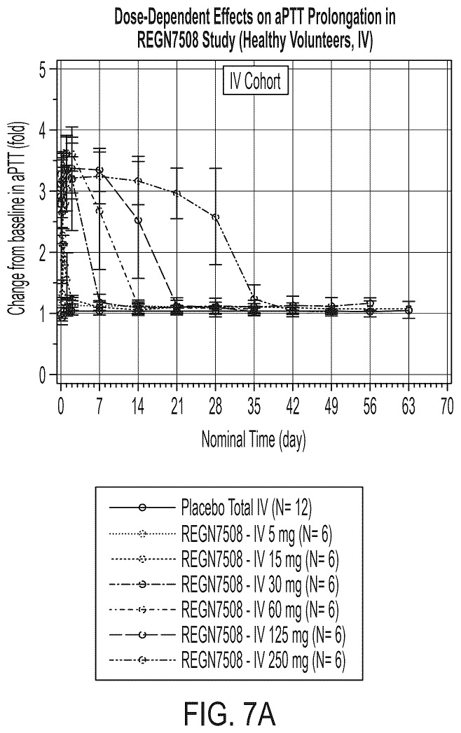

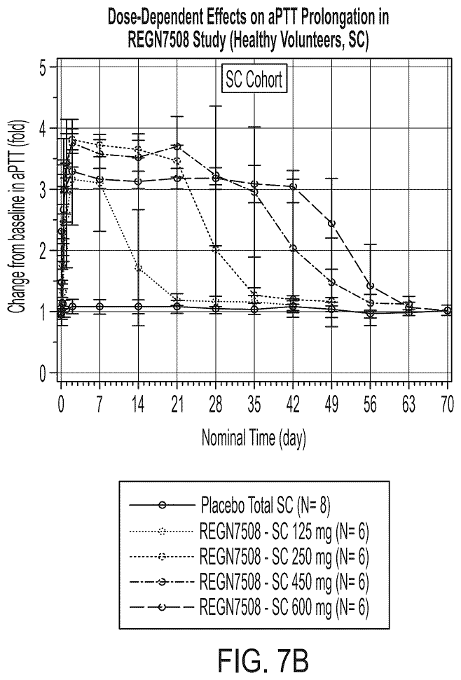

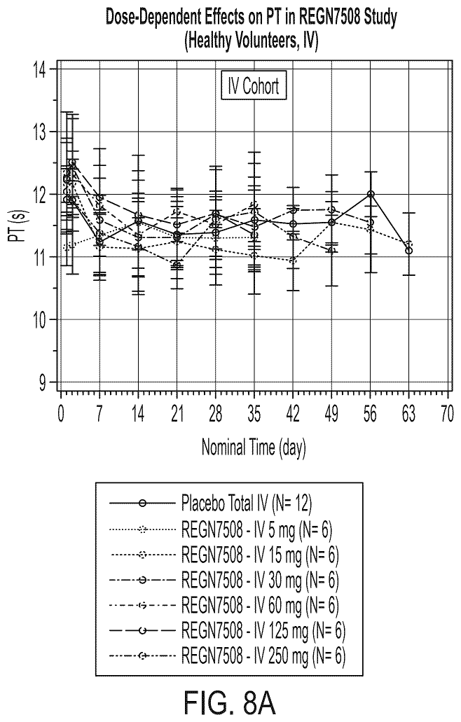

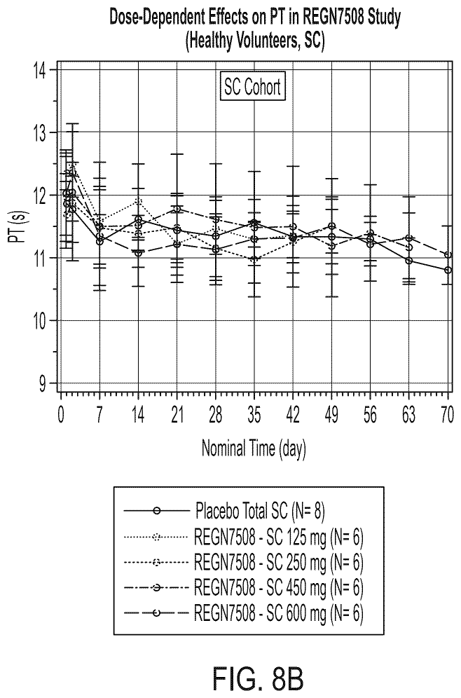

Mean fold change from baseline in aPTT versus time profiles are presented in FIGS. 7A-7B. REGN7508 treatment produced a rapid increase in aPTT; maximum effects occurred for most participants at the first measured timepoint after drug administration. The magnitude and duration of the increase in aPTT was dose-dependent. While the effect at the lowest dose tested (5 mg) was negligible, higher doses produced maximum and progressively longer duration of aPTT increase. At the highest dose for IV (250 mg) and SC (600 mg) administration, the duration of maximal effect was prolonged ≥21 days and ≥42 days, respectively. The mean maximum increase in aPTT ranged from 3.3-fold to 3.8-fold across all cohorts. Mean (±Standard Deviation (SD)) PT vs time profiles at each dose are presented in FIGS. 8A-8B. There were no apparent effects of REGN7508 treatment (IV or SC) on PT at any of the doses tested.

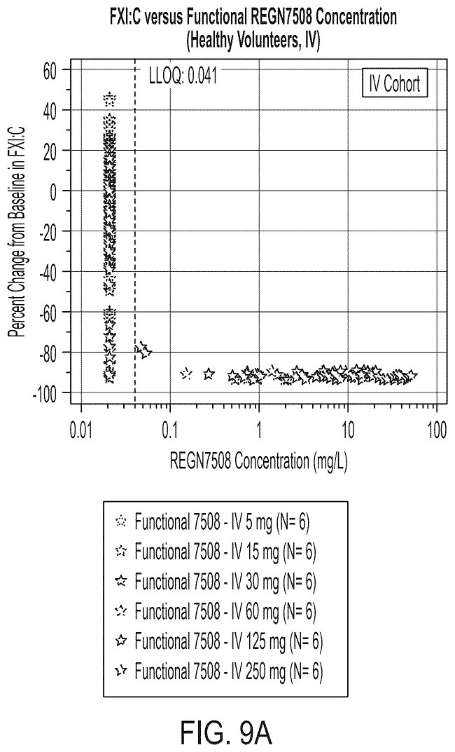

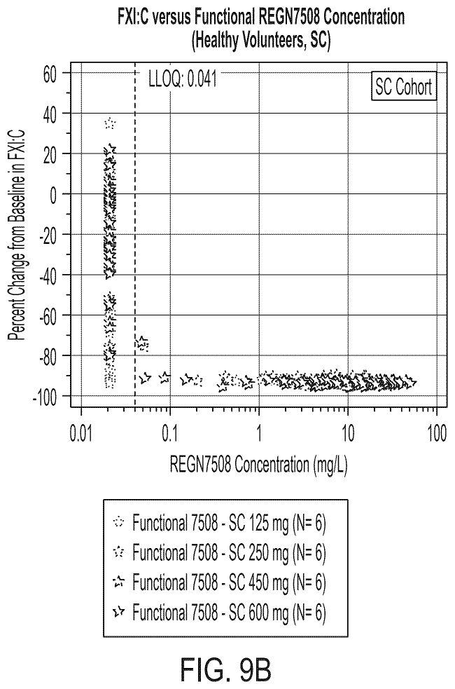

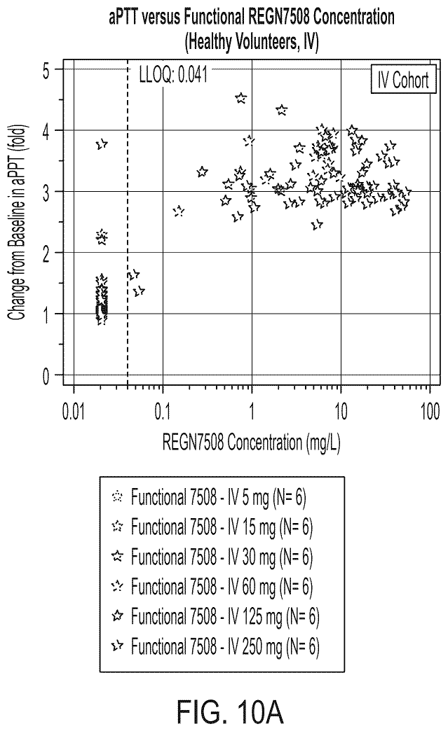

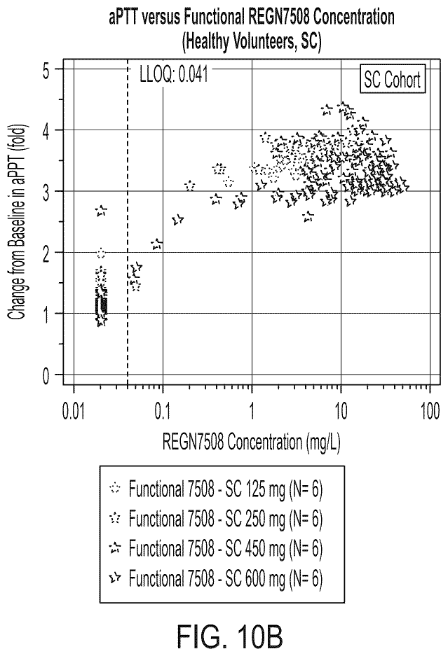

C-R analysis for this study is based on functional REGN7508 concentration in plasma. A scatter plot of mean change from baseline in FXI:C vs concentration of functional REGN7508 (log scale) is shown in FIGS. 9A-9B. The relationship between FXI:C and concentration is steep, with concentration<0.5 mg/L producing maximum measurable suppression of FXI:C (FXI:C assay had a LLOQ of 8% of normal). A scatter plot of fold change from baseline in aPTT vs concentration of functional REGN7508 (log scale) is shown in FIGS. 10A-10B. The relationship between aPTT and concentration is steep, with concentration<2 mg/L producing maximum prolongation of aPTT.

The exploratory biomarker variables in this study were intrinsic-pathway-triggered thrombin generation and extrinsic-pathway-triggered thrombin generation. In TGAs where thrombin generation was induced by EA via the intrinsic pathway in human plasma, REGN7508 increased the lag time for thrombin generation up to 5-fold relative to baseline (no antibody), reduced peak thrombin levels down to 10% of baseline, and reduced endogenous thrombin potential down to 40% of baseline. REGN7508 exerted these effects in a dose-dependent manner with maximal effects achieved at doses of ≥30 mg IV, ≥125 mg SC for REGN7508.

When thrombin generation was induced by TF via the extrinsic pathway, REGN7508 slightly reduced peak thrombin levels down to 90% of baseline and reduced endogenous thrombin potential down to 90% of baseline. REGN7508 exerted these effects in a concentration-dependent manner with maximal effects achieved at concentrations of ≥30 mg IV, ≥125 mg SC for REGN7508. No concentration-dependent increases in lag time for thrombin generation were observed with REGN7508 up to the maximum antibody dose tested (250 mg IV and 600 mg SC for REGN7508).

### Immunogenicity

Two participants from the placebo cohort exhibited pre-existing immunoreactivity to REGN7508. There was no treatment-boosted or treatment-emergent immunogenicity to REGN7508 observed for any participant.

### Interpretation of Clinical Pharmacology Results

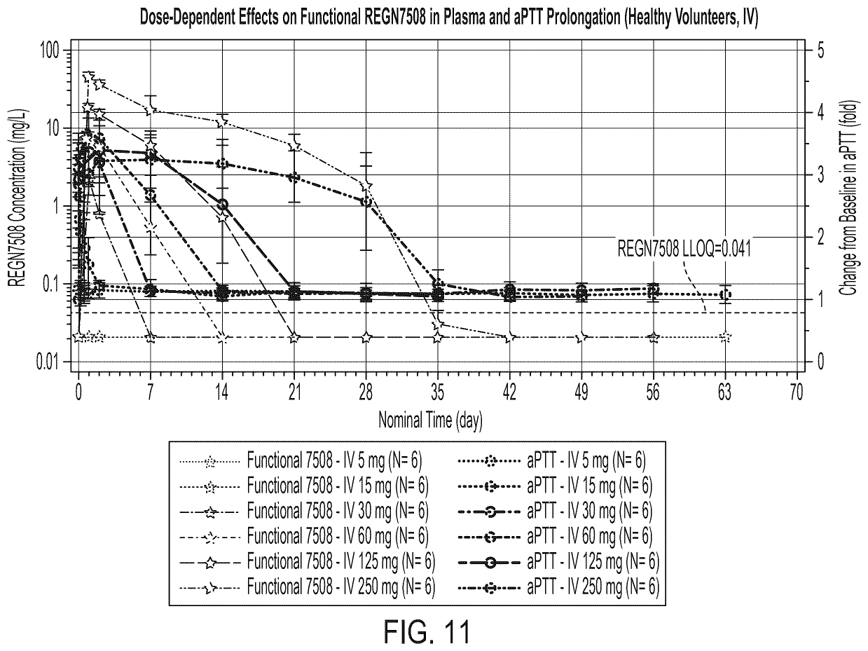

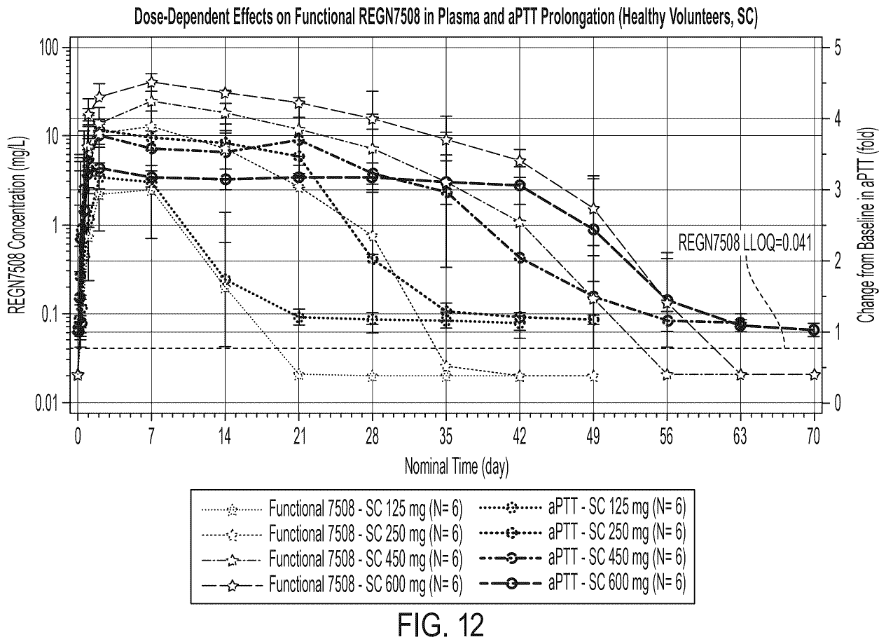

The PD effect runs parallel to the functional REGN7508 concentration, which expresses drug available to bind to its target. FIG. 11 shows the profiles over time of functional REGN7508, along with change from baseline in aPTT following IV dosing of REGN7508>30 mg; FIG. 12 shows the corresponding graph following SC dose administration. The graphs illustrate the difference in the PK profiles of functional REGN7508 concentrations and their relationship with the PD effect of REGN7508.

IV and SC administration reduced FXI:C to undetectable levels; however, due to FXI:C assay limitations (LLOQ 8% of normal activity), it is not possible to determine whether REGN7508 completely suppressed the FXI coagulation. The asymptotic relationship between FXI:C and aPTT with drug concentration indicates that the higher doses of REGN7508 (>250 mg IV or SC) investigated in this study provided optimal inhibition of FXI and can be used in subsequent clinical studies.

### Conclusions

Overall, REGN7508 administered IV or SC was generally well tolerated with no deaths, treatment-emergent SAEs, treatment-emergent AESIs, or TEAEs leading to study discontinuation. The incidence of TEAEs was similar between the total REGN7508 group and the total placebo group. The majority of TEAEs were of mild or moderate severity. Two severe TEAEs were reported (Syncope and Road traffic accident); these occurred in participants treated with placebo. The severe TEAE of Syncope was assessed as related to study drug and the TEAE of Road traffic accident was assessed as not related to study drug. The proportion of participants that experienced treatment-related TEAEs was similar between the total REGN7508 group and the total placebo group. No clinically meaningful safety concerns related to laboratory parameters were identified.

Functional REGN7508 exhibited non-linear PK, indicative of target-mediated clearance. REGN7508 treatment resulted in a dose-dependent increase, up to a maximum of 3-fold, in total FXI concentrations up to a dose of 250 mg IV or 450 mg SC. An apparent decrease was observed in total FXI concentration at the 600 mg SC dose relative to 450 mg; however, the fold change from baseline was higher at 600 mg compared to 450 mg. Dose-dependent reduction in FXI:C and prolongation of aPTT were observed in this study. Maximal PD effects were achieved at relatively low doses; however, the higher doses used in the study (>250 mg IV or SC) achieved prolonged PD effects over 2-4 weeks. No treatment-boost or treatment-effect ADA responses to REGN7508 were observed in any participant.

### Example 6: Randomized, Double-Blind, Placebo-Controlled Study to Evaluate the Safety, Tolerability, Pharmacokinetics, and Pharmacodynamics of Single Ascending Doses of REGN9933 in Healthy Adult Subjects

This randomized, double-blind, placebo-controlled, single-center study was designed to evaluate the safety, tolerability, PK, and PD of single ascending intravenous (IV) and subcutaneous (SC) doses of the human monoclonal antibody (mAb) REGN9933 in healthy adults. The study design using sentinel dosing, sequential cohorts, and planned review of emerging safety and any available PK and PD data allows for progressing through multiple dose levels expeditiously yet safely.

The study population comprised healthy male and permanently amenorrheic female subjects between 18 and 55 years who are at low risk for bleeding and thrombosis. Advanced age is a risk factor for both bleeding and clotting, so a very conservative age range was chosen for adult subjects (Li, 2017) (Yousufuddin, 2019). Women capable of menstruation were excluded since the literature reports that some FXI-deficient women may experience heavy menstruation (menorrhagia) (Kadir, 1999). Subjects requiring medications associated with bleeding risk, including nonsteroidal anti-inflammatory drugs, were excluded. Each participants is required to have a normal PT, aPTT, and platelet count, a negative fecal occult blood test (FOBT), and normal bleeding time test (BTT) prior to randomization to ensure bleeding risk is as low as possible before receiving study drug. All of these design elements were included to limit the risk of bleeding in this first-in-human study.

The primary objective was to evaluate the safety and tolerability of single doses of REGN9933 in healthy subjects. Safety was assessed by the incidences and severities of treatment-emergent adverse events (TEAEs). In addition, several assessments were included in this study to monitor and explore bleeding risk. PT is a standard and validated measure of extrinsic/common pathway activation. Elevated PT following administration of anticoagulation therapies inhibiting the extrinsic/common pathway is associated with an elevated bleeding risk (Woodruff, 2018).

Bleeding time tests and FOBTs were also included in this study as exploratory endpoints and additional assessments of bleeding. Bleeding time tests were measured on day 1 and day 3 to assess bleeding times during anticipated maximal concentration of REGN9933 in the IV and SC cohorts, respectively, and a postdose stool sample is collected no sooner than 3 days after dose administration (to allow for gastrointestinal transit time of red blood cells) but no later than day 8. Flexibility in the timing of stool collection is provided to accommodate each volunteer's habits. Additionally, BTTs and FOBTs were measured at the week 4 follow-up visit to assess any evidence of bleeding risk after prolonged exposure to the study drug. It is anticipated there will be no observed changes in PT, bleeding time, or fecal occult blood in this study, as no changes in these parameters were seen in preclinical studies of REGN9933. Isolated abnormal BTTs and FOBTs were not considered to be actionable safety measures due to poor positive predictive value for clinically significant bleeding events and the inter- and intra-subject variability (Gewirtz, 1996) (Urbas, 2017). However, safety, tolerability, and any bleeding events will be reviewed.

The PD endpoint aPTT is a standard clinically validated measure of intrinsic/common pathway activation. Normal aPTT values are generally between 30 and 40 seconds. In preclinical PK/PD studies in NHPs, REGN9933 increased aPTT approximately 2-fold from baseline over the dose range without increasing PT. In a separate repeat-dose toxicology study in NHPs, REGN9933 also prolonged aPTT with no increase in PT or bleeding times and no detection of fecal blood.

Of note, prolongation of aPTT is not considered a safety signal since elevations are not necessarily associated with bleeding risk (Kitchens, 2005) (Tagariello, 2017). This is especially true in the context of genetic loss and pharmacologic inhibition of the intrinsic pathway (Koch, 2019) (Lorentz, 2019). As discussed above, FXI deficiency is generally associated with minimal spontaneous bleeding risk, although some patients experience increased bleeding after trauma or surgery to sites with high fibrinolytic activity such as the urinary tract, mouth, and nose (Asakai, 1991) (Salomon, 2006). However, REGN9933 exposure is expected to mimic FXII deficiency more closely than FXI deficiency, since REGN9933 blocks activation of FXI by FXIIa but not by other mechanisms. FXII deficiency is characterized by little or no risk of spontaneous or surgical bleeding despite marked prolongation of aPTT (Lammle, 1991) (Girolami, 2004). Notably, Phase 1 studies with three different FXI binding mAbs have been reported. All showed ≥2-fold changes in aPTT at the highest doses, and none reported any bleeding complications (Lorentz, 2019) (Koch, 2019) (Thomas, 2019). In contrast, elevations of aPTT that are due to administration of heparin may be associated with increased bleeding risk due to the inhibition of thrombin by heparin (Alquwaizani, 2013).

The length of the Inpatient Treatment Period was chosen to provide ample time to conduct dense PK/PD sampling and allow for close monitoring of the subjects for AEs and for performance of the BTTs. The length of the Outpatient Follow-Up Period is designed to allow for full characterization of exposure-response relationships, to provide ample time for monitoring for AEs, and to ensure that PD effects are resolved (as judged by aPTT values) for the majority of subjects receiving REGN9933 for at least a week prior to the End-of-Study (EOS). The EOS is no sooner than day 36. For lower dose-level cohorts, it is anticipated PD effects resolve prior to day 29, and EOS will occur on day 36. For higher dose-level cohorts, it is anticipated PD effects will be observed beyond day 36, and the EOS will be extended to ensure subjects are followed until PD effects resolve, as described in the Study Design Description. The Outpatient Follow-Up Period is extended for the entire cohort and not just individuals in order to avoid inadvertent unblinding of study site personnel that might occur if individuals were scheduled for extended follow-up. However, as an additional safety measure, prolonged follow-up may be suggested for individuals.

### Objectives

The primary objective of this study was to evaluate the safety and tolerability of single doses of REGN9933 in healthy adult participants. The secondary objectives included evaluation of the effects of single doses of REGN9933 on intrinsic/common pathway coagulation; evaluation of the effects of single doses of REGN9933 on extrinsic/common pathway coagulation; characterization of the drug (total and functional) concentration profiles and pharmacokinetics (PK) following single escalating doses of REGN9933; characterization of concentration profiles of total and free FXI following single escalating doses of REGN9933; and assessment of the immunogenicity of single doses of REGN9933.

The exploratory objectives of this study are to explore the effects of single doses of REGN9933 on FXI activity; explore the effects of single doses of REGN9933 on intrinsic-pathway-triggered thrombin generation; explore the effects of single doses of REGN9933 on extrinsic-pathway-triggered thrombin generation; explore the effects of single doses of REGN9933 on subclinical gastrointestinal blood loss; explore the effects of single doses of REGN9933 on bleeding time; explore the relationship between indicators of coagulation activity and systemic concentrations of REGN9933; explore the mechanism of action of REGN9933 (related to efficacy and/or safety), the coagulation cascade, and related diseases; and to explore whether potential differences in pharmacodynamics (PD) and safety are associated with genotype and gene expression and to further study FXI and coagulation-related diseases, using whole blood DNA and RNA collected from consented optional substudy participants.

### Study Design

This was a randomized, double-blind, placebo-controlled, single-site first-in-human study of REGN9933. The objective of this study is to evaluate the safety, tolerability, PK, and PD of REGN9933, a monoclonal antibody that blocks coagulation FXI, in healthy adults. This study uses a single ascending dose design. Subjects were randomized in a 6:2 ratio of REGN9933:placebo in up to 9 dose cohorts: 5 intravenous (IV) dose administration, 2 subcutaneous (SC) dose administration, and 2 optional cohorts.

In order to optimize safety, the first two subjects in each cohort (sentinel subjects) were dosed at least 48 hours prior to the remaining subjects in the cohort and are randomized in a 1:1 manner (REGN9933:placebo). Maximal PD effects with IV doses were expected within 48 hours based on results of preclinical PK/PD studies. The remaining subjects in the cohort were dosed only after both sentinel subjects in the cohort had completed at least 48 hours of safety assessments and the data had been reviewed by the investigator and Sponsor with the decision documented by the investigator and the Sponsor. Subcutaneously dosed cohorts may be initiated once all subjects enrolled in an equivalent or higher intravenously dosed cohort have been observed for at least 7 days poststudy drug administration and have completed day 8 (visit 6) safety assessments and after the data have been reviewed at a Dose Escalation Review meeting.

Escalation to the next dose cohort occured once all subjects enrolled in a cohort have been observed for at least 7 days poststudy drug administration and have completed day 8 (visit 6) safety assessments and after the data have been reviewed at a Dose Escalation Review meeting (although screening for the next dose cohort may begin prior to confirmation that the current dose is safe).

The Dose Escalation Review meeting was led by a designated member of the Sponsor clinical team (generally either the Medical Director/Study Director or the clinical trial manager) and at a minimum was attended by Global Patient Safety (GPS) and other individuals, including the investigator and representatives from Clinical Operations, Clinical Pharmacology, and Regulatory Affairs may be included. Additionally, dose escalation decisions were made with input from a Sponsor medical officer with relevant clinical expertise who is not otherwise directly involved in the trial. Any escalation in dose level could not occur until sufficient data (e.g., simulated dose predictions and empiric activated partial thromboplastin time (aPTT) data were available to predict that the next dose level is unlikely to result in a population mean aPTT that is approximately >2× the upper limit of normal (ULN) at 2 months postdose.

### Dose Selection

Single IV doses beginning with 3 mg and ≤11, ≤30, ≤100, and ≤300, as well as single SC doses≤300 mg and ≤600 mg have been selected for study. Dose escalation will occur provided adequate safety/tolerability data without an untoward duration of PD effect is observed at the previous lower doses. A single IV dose of 1000 mg or equivalent SC dose has been identified as the maximum allowed dose that may be used in an optional cohort (or cohorts), assuming adequate safety/tolerability data is observed at lower doses.

Dose selection was informed from the totality of nonclinical pharmacology, safety, PK, and PK/PD studies. PD response was assessed as the fold change from baseline in aPTT values, as preclinical data demonstrate REGN9933 elevates aPTT without any measurable impact on other coagulation parameters such as PT and bleeding time. Additionally, available clinical data suggest antithrombotic effects of FXI inhibition occur with maximal prolongation of aPTT in a safe and well-tolerated manner (Lorentz, 2019). Preclinical studies were conducted in NHPs because NHPs were identified as a clinically relevant species based upon similarities in the FXI amino acid sequence and plasma concentration. Furthermore, REGN9933 binding affinity in NHPs is similar to that in humans.

Systemic concentration of REGN9933 and aPTT data from two NHP PK/PD studies (R3448-PK-19085 and R9933-PK-20098), together with additional REGN9933 concentration data from a Good Laboratory Practice (GLP) NHP toxicology study (R9933-TX-20070), were used to build PK/PD models of REGN9933 in NHPs, which described the concentration of REGN9933 in serum and its relationship with changes in aPTT using a standard Hill equation (Wagner, 1968). Human PK profiles were predicted from the observed PK profile in NHPs via a standard allometric scaling approach (Dong, 2011). As in vitro plasma aPTT assays suggested differences in potency between humans and NHPs (R9933-PH-20164), PK/PD models were constructed under two different assumptions:
(i) the concentration-response relationship in normal healthy volunteers (NHVs) is the same as that observed in NHP PK/PD studies, or
(ii) the concentration-response relationship in NHVs is the same as that observed in the human plasma aPTT assay.

Models incorporating these assumptions were used to compute a range of expected aPTT response profiles in NHVs. The maximum-fold change from baseline in aPTT (Eₘₐₓ) values was predicted to be 2-fold under assumption (i) and 2.6-fold under assumption (ii). Similarly, the concentration of REGN9933 needed to achieve 50% of the maximum effect was estimated to be 14 mg/L under assumption (i) and 2.1 mg/L under assumption (ii). Using simulations from the modeling described above, an IV dose of 3 mg in NHVs is predicted to yield a peak concentration (Cₘₐₓ) of 0.830 mg/L, a maximum aPTT fold change of 0.1% of Eₘₐₓ under assumption (i), and a maximum aPTT fold change of 24% of Eₘₐₓ under assumption (ii). The aPTT effect at this dose is anticipated to result in no detectable effect under assumption (i) and immediately decline from its maximum value and return to within 10% of baseline within 2 weeks under assumption (ii) and therefore is considered to represent a minimal PD effect in human subjects.

Based on the above assessment, a starting dose of 3 mg REGN9933 administered as a single IV infusion was selected for this first-in-human (FIH) clinical study. Dose escalation will progress at no more than half-log intervals (approximately 3-fold) up to a maximum of 1000 mg (which may be used in an optional cohort). Additional simulations from the PK/PD models suggest a single IV dose of 10 mg is expected to result in an aPTT profile that transiently achieves 2% of Eₘₐₓ under assumption (i) and 58% of Eₘₐₓ under assumption (ii) before returning to baseline. Subsequent nominal IV dose levels of 30 mg and 100 mg are expected to result in aPTT profiles that briefly achieve 22% and 84% of Eₘₐₓ, respectively, under assumption (i) or 85% and 96% of Eₘₐₓ, respectively, under assumption (ii) before returning to baseline. The 300 mg IV dose and the 1000 mg IV dose (the maximum allowed dose) are expected to achieve near-maximal aPTT prolongation and are anticipated to maintain aPTT elevations>10% above baseline values for 8 and 17 weeks, respectively, under assumption (i) or 20 and 30 weeks, respectively, under assumption (ii). Single SC doses of 300 and 600 mg are predicted to achieve near-maximal aPTT prolongation and to achieve and maintain aPTT elevations>10% above baseline values for 6 and 10 weeks, respectively, under assumption (i) or 18 and 23 weeks, respectively, under assumption (ii). Attainment of Eₘₐₓ will be inferred when no subsequent increase in aPTT is observed in at least one escalated dose (i.e., when a plateau in the exposure-response relationship is observed). The goal of dose escalation is to fully explore exposure-response relationships and to define Eₘₐₓ while minimizing exposure of volunteers to REGN9933.

The doses tested in the FIH study were supported by the results of the GLP 5 week repeat-dose and 13 week recovery toxicology study in NHPs. REGN9933 was well-tolerated with a no-observed-adverse effect level (NOAEL) of 100 mg/kg IV. The exposure observed at the NOAEL in the NHPs was over 7,000-fold (based on Cₘₐₓ) and over 21,000-fold (based on area under the curve [AUC]) greater than those predicted in humans for the starting dose of 3 mg IV. As noted, the maximum allowed dose in this study is 1000 mg IV and may be used in an optional cohort (or cohorts), assuming adequate safety/tolerability data is observed at lower doses. Using the predicted human PK, the calculated exposure at the maximum planned dose of 1000 mg are a Cₘₐₓ of 277 mg/L and an AUC_{inf} of 8270 day mg/L. The NOAEL exposure levels are therefore over 20-fold greater (either based on Cₘₐₓ or AUC).

### Study Duration

The minimum study duration clinical follow-up (the last in-person visit) for each participant was approximately 36 days, including the 3 day Inpatient Treatment Period and at least 33 days in the Outpatient Clinic Follow-Up Period, followed by a day 100 telephone call for all participants which will be the end of study. If the length of the Outpatient Clinic Follow-up is more than 100 days, then the questions from the telephone call (regarding pregnancy and SAEs) can be answered in-person at the last Outpatient Clinic Follow-up visit.

If 3 or more participants in a given cohort have aPTT values that are >10% above the ULN on the day 29 visit, then the Outpatient Clinic Follow-Up Period is extended, and all participants in the cohort continue to return to the study site every 7±2 days for follow-up visits. The Extended Follow-Up continues until fewer than 3 of the participants in the cohort have aPTT values that are >10% above the ULN. Once this occurs, all participants in the cohort return 7±2 days later for the End of Outpatient Clinic Follow-up (EOF) visit. At the higher doses, total follow-up may be 9 weeks or longer.

Subcutaneously-dosed cohorts will be initiated only after an equivalent or higher IV dose level has been evaluated. Therefore, for SC-dosed cohorts only, the timing of the EOS visit may be determined based on analysis of aPTT data from completed IV cohorts. Total exposures are expected to be less with SC dosing than IV dosing at the same dose level. Therefore, resolution of aPTT prolongation will occur earlier in SC cohorts compared to the same dose level in IV cohorts. Timing of the EOS visits in the SC cohorts will be chosen in advance such that aPTT will have normalized.

There will be 2 pre-specified SC cohorts. One SC cohort will test a dose level of 100 mg REGN9933/placebo; the EOS visit for this SC cohort will occur on day 36. The other SC cohort will test a dose level of 300 mg REGN9933/placebo; the EOS visit for this SC cohort will occur on or before day 64. Sentinel dosing is required for all cohorts, including these SC cohorts.

At the discretion of the investigator and the Sponsor, individual participant follow-up may be further extended based on the need to monitor aPTT or extend the safety evaluation.

Each participant will have a day 100 telephone call to ensure compliance with the contraception rules as well as to record any new SAEs.

### End of Study

The end of study is defined as the date the last participant completes the end of last in-person or telephone visit, withdraws from the study, or is lost to follow-up (i.e., the study participant can no longer be contacted by the investigator.

### Population

Up to 56 adult participants will be randomized in up to 5 sequential, ascending single IV dose cohorts and 2 sequential, ascending single SC dose cohorts. An additional 2 optional cohorts may be exercised. Up to 72 participants may be randomized if the optional cohorts are conducted.

The study population will consist of healthy males and females (postmenopausal or permanently unable to menstruate due to a surgical procedure or other medical condition), 18 to 55 years of age (inclusive), with low risk for bleeding or thrombosis based on medical history and physical and laboratory evaluations.

### Inclusion Criteria

A participant must meet the following criteria to be eligible for inclusion in the study:
Male or female 18 to 55 years (inclusive) at the screening visit;
Body mass index between 18.0 and 32.5 kg/m² (inclusive) at the screening visit;
Judged by the investigator to be in good health based on medical history, physical examination, vital sign measurements, and ECGs performed at screening and/or prior to administration of initial dose of study drug;
Participant is in good health based on laboratory safety testing obtained at the screening visit and/or prior to administration of initial dose of study drug; Participant with suspected or confirmed Gilbert's disease can be enrolled in the study;
Normal aPTT, normal PT, and normal platelet counts at screening period and at the day −1 visit as defined by the local laboratory;
Hemoglobin value≥11.0 g/dL for females and ≥12.9 g/dL for males at the screening and day 1 visits;
Negative FOBT during screening period;
Normal BTT at day −1 as defined by the study site;
Participant is willing and able to comply with clinic visits and study-related procedures; and
Provide informed consent signed by study participant.

### Exclusion Criteria

A participant who met any of the following criteria was excluded from the study:
History of any major surgical procedure or clinically significant physical trauma, in the opinion of the investigator, that may pose a risk to the participant by study participation;
Whole blood donation within the previous 56 days or plasma donation within the previous 7 days prior to the screening visit;
Members of the clinical site study team and/or his/her immediate family, unless prior approval granted by the Sponsor;
Pregnant or breastfeeding women;
Women of childbearing potential (WOCBP), defined as women who are fertile, following menarche until becoming postmenopausal, unless permanently surgically sterile. The only allowed permanent sterilization methods for this study are hysterectomy and/or bilateral oophorectomy;
A postmenopausal state is defined as no menses for 12 months without an alternative medical cause. A high follicle-stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to the Clinical Trial Facilitation Group guidance;
Sexually active men with WOCBP partners who are unwilling to use the following forms of medically acceptable birth control during the study through end of study visit: vasectomy with medical assessment of surgical success OR consistent use of a condom;
History of clinically significant cardiovascular, respiratory, hepatic, renal, gastrointestinal, endocrine, hematological, psychiatric, neurological, or dermatologic disease, as assessed by the investigator, that may confound the results of the study or poses an additional risk to the participant by study participation. This includes but is not limited to history of thrombotic events, known bleeding disorders, iron-deficiency anemia, central nervous system bleeding, and gastrointestinal bleeding including that caused by hemorrhoids or anal fissures;
Presents any concern to the study investigator that might confound the results of the study or poses an additional risk to the participant by their participation in the study;
Hospitalized for any reason within 30 days of the screening visit;
Using the Modification of Diet in Renal Disease equation has an estimated glomerular filtration rate (GFR) of <60 mL/min/1.73 m² at screening;
Note: If participant has a GFR below 60 mL/min/1.73 m², one repeat test is allowed. Participant may be enrolled if repeat test demonstrates GFR of >60 mL/min/1.73 m²;
Current smoker or former smoker, including e-cigarettes, who stopped smoking within 12 months prior to the screening visit;
Confirmed positive drug test result at the screening visit and/or prior to randomization or a history of drug abuse within a year prior to the screening visit;
History of alcohol abuse within the last 2 years prior to the day 1 visit;
Is positive for human immunodeficiency virus (HIV) or hepatitis B surface antigen (HBsAg) at the screening visit. Evidence of prior hepatitis B immunization or prior resolved hepatitis B infection is not an exclusion;
Is positive for hepatitis C antibody and if so, positive for qualitative (i.e., detected or not detected) hepatitis C virus RNA test at the screening visit;
Any malignancy, except for nonmelanoma skin cancer or cervical/anus in situ, that have been resected with no evidence of metastatic disease for 3 years prior to the screening visit
History of significant multiple and/or severe allergies (e.g., latex gloves) or has had an anaphylactic reaction to prescription or nonprescription drugs or food;
Participated in any clinical research study evaluating another investigational drug including biologics or therapy, including specific immunotherapy, within 90 days or at least 5 half-lives (whichever is longer) of an investigational biologic drug or at least 4 weeks for other investigational drug prior to the screening visit;
Use of any prescription and nonprescription medications or nutritional supplements from approximately 2 weeks or 5 half-lives, whichever is longer, prior to first administration of the study drug through the EOF except the permitted medications listed. This includes anticoagulants and antiplatelets, including NSAIDs or salicylic acid; or
Unwilling or unable to comply with the permitted and prohibited medications specifications for this study.

### Treatments

REGN9933 drug product is supplied as a 265 mg lyophilized powder in sterile single-use vials. Placebo to match REGN9933 is supplied as a lyophilized powder in single sterile-use vials.

Participants will be randomized in a 6:2 ratio of REGN9933:placebo in up to 9 dose cohorts: 5 IV dose administration cohorts; 2 SC dose administration cohorts; 2 optional cohorts.

Intravenous dose levels will start at 3 mg. Doses for subsequent cohorts will increase at no more than half-log increments. The SC dose levels tested will be 100 mg and 300 mg. The maximum dose in this study that may be used in an optional cohort is 1000 mg IV, assuming adequate safety/tolerability data is observed at lower doses.

### Endpoints

The primary endpoint is the incidences and seventies of treatment-emergent adverse events (TEAE) in participants treated with REGN9933 or placebo. The secondary endpoints are: change from basement in aPTT through EOS visit; change in baseline in prothrombin time (PT) through the EOS visit; concentrations of total REGN9933 in serum and functional REGN9933 in plasma through the EOS visit; change from baseline in total FXI concentrations through the EOS visit; and incidence of antidrug antibodies to REGN9933 overtime.

### Procedures and Assessments

No formal efficacy assessments were be performed. Safety assessments included monitoring vital signs (semi-recumbent blood pressure, pulse, temperature, and respiration), physical examination, ECG, fecal occult blood test (FOBT), bleeding time test (BTT), laboratory evaluations (PT, hematology, chemistry, and urinalysis), and the monitoring and reporting of adverse events (AEs). Pharmacodynamics assessments were made using measurements of aPTT. Samples were collected for drug concentration, ADA incidences, concentration of total target, and exploratory assessments.

Blood samples were optionally collected and/or stored for future research and/or pharmacogenomics substudies for participants who choose to participate in the optional components of the main study.

Activated partial thromboplastin time (aPTT), Factor XI functional activity, and Thrombin Generation Assays (TG) were discussed in Example 5.

No formal statistical hypothesis testing were carried out in this dose escalation study.

### Results

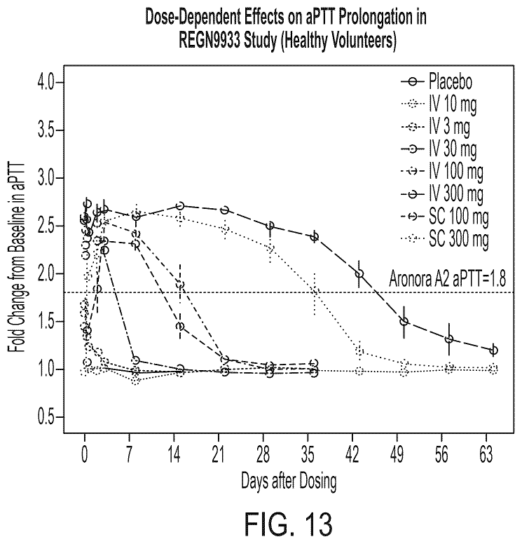

40 healthy adult men and post-menopausal and/or surgically sterile women have been randomized in this ongoing clinical study across 5 IV-dosed cohorts, and all of these 40 study participants have completed end-of-study (EOS) visits. Each cohort contained 8 subjects (6 randomized to REGN9933 and 2 randomized to placebo) at the following dose levels: 3 mg, 10 mg, 30 mg, 100 mg, and 300 mg. All subjects in the first 4 IV dose level cohorts (3, 10, 30, and 100 mg REGN9933/placebo) were able to complete the EOS visit on day 36. All participants in the last IV-dosed cohort (300 mg REGN9933/placebo) had extended outpatient follow-up visits after day 36 and had aPTT values return to ≤10% above the upper limit of normal (ULN) by approximately day 64 (FIG. 13).

No adverse events (AEs) have been reported as related to study drug. No severe nor serious AEs have been reported.

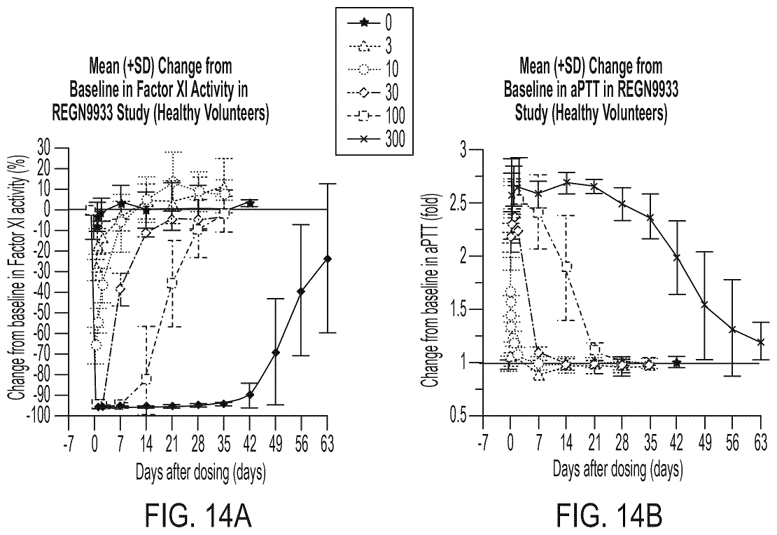

After administration of ≥100 mg IV REGN9933, FXI activity was reduced by >80% and returned to within 10% of baseline by day 35 for all doses<300 mg IV, and within 20% of baseline by day 64 for the 300 mg IV dose, see FIG. 14A.

A dose-dependent aPTT prolongation has been observed across these 5 IV-dosed cohorts with an aPTT mean fold-change from baseline of approximately 2.8 in subjects receiving 300 mg IV of REGN9933 (FIG. 14B). More specifically, in clotting assays, REGN9933 increased aPTT relative to baseline (i.e., no antibody) in a concentration-dependent manner; increases of up to 2.5-fold in plasma were observed at the range of doses tested (REGN9933: 10-300 mg IV, 100-300 mg SC), see FIG. 13. The 2-fold increase of aPTT relative to baseline was estimated to occur at ≥30 mg IV, ≥100 mg SC for REGN9933 in participant plasma. Following the single dose administration of each subject anti-FXI monoclonal antibody, aPTT values returned to within 10% of normal by Day 64. No change in PT relative to baseline was observed up to the maximum antibody concentration tested.

In TGAs where thrombin generation was induced by EA via the intrinsic pathway in human plasma, REGN9933 increased the lag time for thrombin generation up to 5-fold relative to baseline (no antibody), reduced peak thrombin levels down to 10% of baseline, and reduced endogenous thrombin potential down to 40% of baseline. REGN9933 exerted these effects in a dose-dependent manner with maximal effects achieved at doses of ≥30 mg IV, ≥100 mg SC.

When thrombin generation was induced by TF via the extrinsic pathway, REGN9933 slightly reduced peak thrombin levels down to 90% of baseline and reduced endogenous thrombin potential down to 90% of baseline. REGN9933 exerted these effects in a concentration-dependent manner with maximal effects achieved at concentrations of ≥30 mg IV, ≥100 mg SC. No concentration-dependent increases in lag time for thrombin generation were observed with REGN9933 up to the maximum antibody dose tested (300 mg IV and SC for REGN9933).

### Pharmacokinetics and Pharmacodynamics

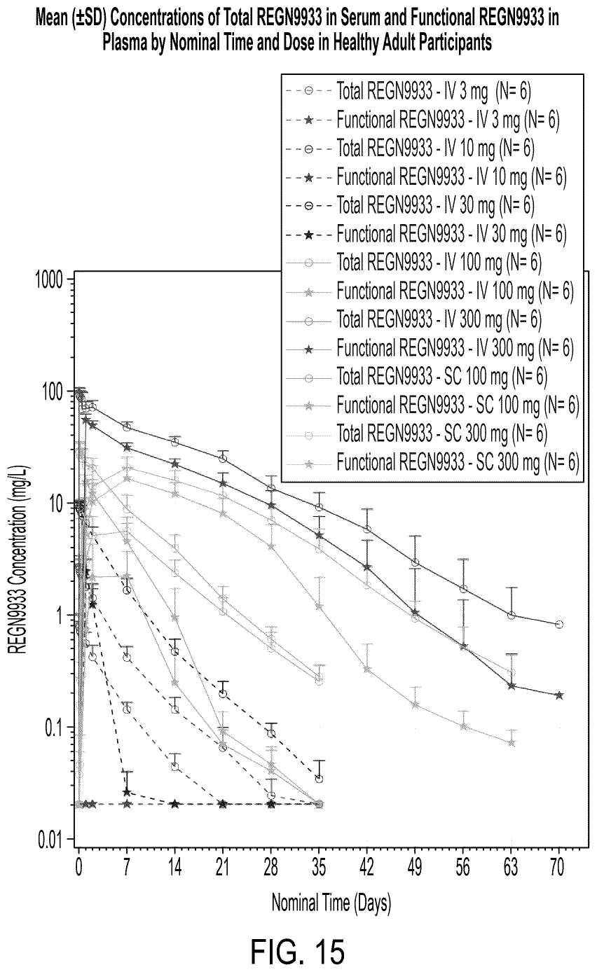

The concentration-time profiles of total REGN9933 in serum and functional REGN9933 in plasma following IV administration are presented in FIG. 15. Functional REGN9933 represents antibody with one or two free binding sites, available to bind its target (Factor XI). Total REGN9933 represents all antibody, regardless of binding status. Following the initial distribution phase, the rate of decline of concentration for the lower IV dose groups was faster in comparison to the 300 mg IV cohort. This is consistent with target-mediated elimination, suggesting some saturation of the target-mediated elimination pathway at the highest IV dose. Following SC administration, maximum concentrations of REGN9933 were observed approximately 7 days after dosing (FIG. 15).

Consistent with the target-mediated kinetics displayed by the concentration-time profiles, the PK of REGN9933 was described as nonlinear. This nonlinear behavior was evident from the greater than dose-proportional increase in the mean AUC values, along with a decrease in CL, observed over the dose range studied (3 mg 1 to 300 mg IV). Following IV administration, Cmax increased in an approximately dose proportional fashion (Table 9 and Table 10).

Similar to IV administration, nonlinearity was observed following SC administration of REGN9933 100 or 300 mg, with AUC values increasing more than proportionally to dose and CL/F decreasing with dose. Cₘₐₓ increased approximately dose proportionally for total REGN9933 and more than dose proportionally for functional REGN9933 following SC administration (Tables 9 and 10).

TABLE 9
Mean (±SD) Pharmacokinetic Parameters Calculated from Concentrations of Total REGN9933
in Serum by Treatment Group in Healthy Adult Participants (Study R9933-HV-2107, PKAS)

| | IV | | | | | SC | |
|---|---|---|---|---|---|---|---|
| | 3 mg | 10 mg | 30 mg | 100 mg | 300 mg | 100 mg | 300 mg |
| | (N = 6) | (N = 6) | (N = 6) | (N = 6) | (N = 6) | (N = 6) | (N = 6) |
| Parameter (Units) | Mean (SD) | Mean (SD) | Mean (SD) | Mean (SD) | Mean (SD) | Mean (SD) | Mean (SD) |
| AUCₗₐₛₜ (day*mg/L) | 2.95 (0.764) | 10.3 (2.38) | 39.1 (7.87) | 185 (45.7) | 1255 (200.00) | 78.6 (23.5) | 466 (133) |
| AUC_{inf} (day*mg/L) | 3.24 (0.713) | 10.7 (2.44) | 39.6 (7.83) | 187 (45.9) | 1264 (205.00) | 81.1 (24.1) | 469 (135) |
| CL for IV or CL/F for SC | 0.960 (0.196) | 0.983 (0.268) | 0.780 (0.136) | 0.560 (0.126) | 0.243 (0.039) | 1.33 (0.401) | 0.698 (0.259) |
| (L/day) | | | | | | | |
| Cₘₐₓ (mg/L) | 0.799 (0.229) | 2.73 (0.661) | 9.30 (1.26) | 29.8 (6.08) | 98.8 (13.0) | 6.04 (2.06) | 20.9 (5.06) |
| Tₘₐₓ (day) | — | — | — | — | — | 5.34 (2.59) | 6.17 (2.04) |
| Vss for IV or Vz/F for SC (L) | 4.88 (0.960) | 5.29 (1.01) | 4.48 (0.753) | 4.17 (0.854) | 3.59 (0.457) | 12.6 (3.98) | 7.96 (3.19) |

TABLE 10
Mean (±SD) Pharmacokinetic Parameters Calculated from Concentrations of Functional REGN9933
in Serum by Treatment Group in Healthy Adult Participants (Study R9933-HV-2107, PKAS)

| | IV | | | | | SC | |
|---|---|---|---|---|---|---|---|
| | 3 mg | 10 mg | 30 mg | 100 mg | 300 mg | 100 mg | 300 mg |
| | (N = 6) | (N = 6) | (N = 6) | (N = 6) | (N = 6) | (N = 6) | (N = 6) |
| Parameter (Units) | Mean (SD) | Mean (SD) | Mean (SD) | Mean (SD) | Mean (SD) | Mean (SD) | Mean (SD) |
| AUCₗₐₛₜ (day*mg/L) | — | — | 3.52 (2.22) | 78.3 (26.8) | 774 (134) | 20.3 (15.3) | 312 (101) |
| AUC_{inf} (day*mg/L) | — | — | | 78.6 (26.8) | 775 (135) | 23.6 (15.1) | 313 (101) |
| CL for IV or CL/F for SC (L/day) | — | — | | 1.43 (0.589) | 0.396 (0.0650) | 7.23 (7.03) | 1.09 (0.529) |
| Cₘₐₓ (mg/L) | — | — | 2.45 (0.698) | 15.3 (2.69) | 55.3 (7.39) | 2.73 (1.79) | 16.5 (4.23) |
| Tₘₐₓ (day) | — | — | — | — | — | 3.67 (2.59) | 7.01 (0.008) |
| Vss for IV or Vz/F for SC (L) | — | — | — | 6.43 (1.31) | 5.35 (0.622) | 45.8 (40.3) | 21.9 (14.6) |

IV = intravenous; N = Number of participants in each treatment group; PKAS = Pharmacokinetic Analysis Set; SC = subcutaneous; SD = Standard deviation
Only SC cohorts were included for Tₘₐₓ

REGN9933 treatment produced a rapid decrease in FXI activity (FXI:C), and maximum effects occurred on average at the first measured timepoint after IV drug administration at 12 hours post-dose or at 2 to 7 days post-dose following SC administration. The magnitude and duration of the decrease in FXI:C was REGN9933 dose-dependent. While the effect at the lowest IV dose tested (3 mg IV) was limited, higher IV doses (≥30 mg IV) resulted in near complete suppression of FXI:C. The LLOQ of the FXI:C assay was 5% FXI activity. At the highest dose of 300 mg IV, the maximal reduction in FXI:C was maintained in all participants for ≥35 days. The 300 mg SC dose produced a similar magnitude of effect on FXI:C but for a shorter duration compared to IV administration. At the 100 mg dose level, both IV and SC administration resulted in similar magnitude and duration of effect on FXI:C (FIG. 14A).

REGN9933 treatment produced a rapid increase in aPTT, with levels increasing to >2-fold baseline by the first measured timepoint after IV drug administration at 0.5 hours post-dose for the 30 mg to 300 mg cohorts and at 1 to 7 days post-dose after SC administration for the 100 mg and 300 mg cohorts (FIG. 14B). The magnitude and duration of the increase in aPTT were REGN9933 dose-dependent. While the effect at the lowest IV dose tested (3 mg) was negligible, higher IV doses produced marked increases in aPTT. At the highest IV dose tested (300 mg), the duration of the effect was prolonged and near-maximal increases in aPTT were maintained in all participants for ≥21 days. At corresponding dose levels, SC administration of REGN9933 resulted in a similar magnitude of effect on aPTT with a modestly shorter duration and greater variability relative to IV administration. The maximum observed aPTT in any individual at any dose was 85.6 seconds.

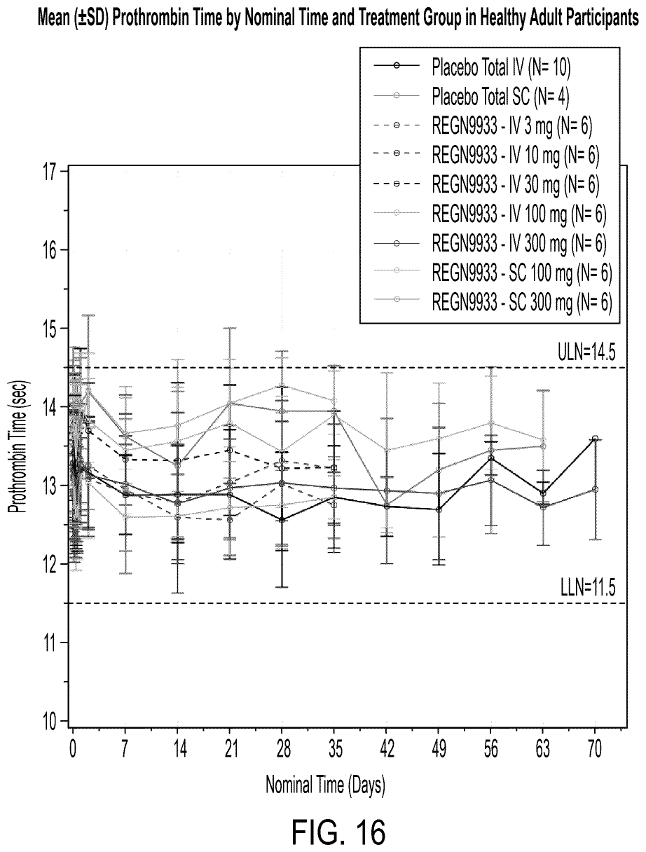

There were no apparent effects of REGN9933 treatment on PT at any of the doses tested (FIG. 16).

### Example 7: A Parallel Group Study in Healthy Participants to Quantitate Increases in Subclinical Gastrointestinal Blood Loss Following Administration of Aspirin Alone or in Combination with Rivaroxaban or REGN9933 or REGN7508

The goal of the study was to compare subclinical gastrointestinal (GI) bleeding following administration of rivaroxaban combined with aspirin to aspirin alone, and to aspirin combined with REGN9933 and REGN7508.

The study comprised a 1-week baseline period (on no study treatment) followed by 2 weeks on a study treatment regimen. This baseline-controlled design allowed for more robust comparisons between groups because of normalization of treatment effects on fecal hemoglobin content (FHC) to a given individual's baseline values. Based on extrapolation from published studies involving high dose Non-steroidal anti-inflammatory drugs (NSAIDs) and/or aspirin and from study R0000-HV-2229 in healthy volunteers, the equivalent of <1 mL blood per day was expected at baseline, with an estimated increase of 0 to 5 mL/day on the study medication regimen.

Healthy volunteers without risk factors for bleeding complications (as judged by history, physical exam, and laboratory evaluation) were included to minimize risk. Consistent with a healthy population, participants also must have had an estimated glomular filtration rate (eGFR) of ≥60 mL/min/1.73 m². This range was intended to identify healthy participants, not those with underlying renal disease. There was no requirement for modification of rivaroxaban dose in this population. Healthy volunteers allowed for assessment of bleeding without the complications of the medical issues present in a patient population. Alcohol was prohibited during the study to avoid risk of bleeding due to alcohol-related gastritis.

HemoQuant is an established chemical method for determining quantitative subclinical heme content in the stool, measured as FHC, and is sensitive to both proximal and distal GI bleeding (Schwartz, 1983). HemoQuant has the advantage of not requiring radioactive tracers, unlike 51-chromium RBC labeling, a traditional method for quantitative GI blood loss assessment. HemoQuant has been reported to be more sensitive than 51-chromium (Schwartz, 1983) and to be able to quantify subtle increases in GI blood loss in patients taking aspirin compared to no aspirin (Greenberg, 1996) or in patients with GI lesions as compared to healthy controls (Ahlquist, 1985). FHC as judged by HemoQuant in healthy volunteers are low (<1 mg hemoglobin/g stool) with a minimal inter-subject variability (SD<1 mg/g). HemoQuant was performed on the 2 weekly stool sample collections to allow for sufficient data to assess reproducibility within participants, to average out sampling error, and to improve power to detect changes induced by study medications while not unduly burdening volunteers.

Four arms were included, and participants received an aspirin regimen likely to result in measurable subclinical GI bleeding, dosed either as monotherapy, or in combination with rivaroxaban, REGN7508, or REGN9933. The goal was to measure subclinical GI bleeding associated with rivaroxaban combined with aspirin compared to aspirin alone, and to aspirin combined with REGN9933 or REGN7508. The aspirin and rivaroxaban regimens were designed with consideration of clinical relevance, safety, and yet enough evidence of association with bleeding events in patient data such that increases in subclinical GI blood loss are likely. All the arms include aspirin, which is known to damage mucosa of intestinal track and predispose to subclinical blood loss. The aspirin alone arm defined blood levels in the absence of anticoagulants.

Rivaroxaban, a DOAC, was added in one of the arms as there are multiple clinical scenarios where an anticoagulant may be added to aspirin. This arm includes full dose (20 mg QD) rivaroxaban, a dose indicated for stroke prevention in atrial fibrillation.

The duration of the treatment period (2 weeks) was chosen based on literature reports that subclinical GI blood loss increases for at least 2 weeks after the initiation of study medications (Hunt, 2000) (Lynch, 1989) and based on data from study R0000-HV-2229 where increases in FHC were observed following a 2-week treatment period with either aspirin alone or aspirin combined with rivaroxaban.

The study was open-label. The need for blinding to group assignment was judged to be minimal as the primary endpoint assessment was a laboratory evaluation of stool, which was performed by personnel blinded to group assignment. The sponsor was unblinded.

Failure of volunteers to properly take study medication can reduce potential changes in FHC, which is an important factor in interpreting the data. Therefore, participant adherence was assessed by pill diaries and pill counts at visit 4.

The sample size per arm was up to approximately 56 participants. This sample size provided 80% power to detect a 35% increase in FHC as measured by HemoQuant assay, which was the observed difference in FHC change from baseline between rivaroxaban plus aspirin and aspirin alone in study R0000-HV-2229. Approximately 10% of the participants enrolled were first-generation Japanese to support further clinical investigation in Japanese participants as part of the future global development of REGN9933 and REGN7508.

### Objectives

The primary objective of this study is to compare subclinical GI bleeding following administration of rivaroxaban combined with aspirin to 1) aspirin alone, 2) REGN9933 combined with aspirin, and 3) REGN7508 combined with aspirin. The secondary objectives include evaluation of the safety and tolerability of REGN9933 and REGN7508 when combined with aspirin (evaluated alongside aspirin alone and aspirin plus rivaroxaban); evaluation of the major and non-major bleeding risk of REGN9933 and REGN7508 when administered with aspirin; evaluation of the PK of REGN9933; evaluation of the PK of REGN7508; assessment of the PD effects of REGN9933 and REGN7508 on intrinsic and extrinsic coagulation pathways; assessment of the immunogenicity of REGN9933; and assessment of the immunogenicity of REGN7508. The exploratory objectives of this study are to explore the minor bleeding risk of REGN9933 and REGN7508 when administered with aspirin; explore whether REGN9933 or REGN7508 increases subclinical GI blood loss in participants receiving aspirin compared to their own baseline; determine rates of GI blood loss over time; explore biomarkers related to FXI inhibition by REGN9933 and REGN7508; and study REGN9933 and REGN7508 mechanism of action and biology related to efficacy and/or safety, the coagulation cascade, and related diseases

### Study Design

This study is a randomized open-label parallel group trial in healthy volunteers at low risk for bleeding. Key inclusion/exclusion criteria included generally good health, no history of anemia, no bleeding diathesis, nor GI pathology. Use of medications that may promote GI bleeding (such as non-steroidal anti-inflammatory drugs) or which may interfere with metabolism of rivaroxaban were prohibited during the study and for 2 weeks or 5 half-lives, whichever is longer, prior to first administration of the study treatments. Screening (visit 1) included confirming inclusion/exclusion criteria, full medical history, and physical and laboratory evaluations. Following screening (days −30 to −1), the study included an approximate 1-week baseline period (days 1 to 7) followed by a 2-week treatment period (days 8 to 21) involving exposure to aspirin either as monotherapy or in combination with either single dose REGN9933 300 mg IV, single dose REGN7508 250 mg IV, or rivaroxaban 20 mg once daily orally. On day 8, participants were randomized 1:1:1:1 to 1 of 4 different treatment arms. Randomization was stratified by Japanese versus Non-Japanese. Approximately 10% of the participants enrolled were first-generation Japanese. Stool samples were collected twice a week (at least 1 day apart) throughout the baseline and treatment periods for the primary endpoint analysis of quantitative fecal hemoglobin with HemoQuant, a chemical laboratory test of FHC. In addition to the screening visit, there was a clinic baseline visit (visit 2) at study initiation (day 1) for reconfirming inclusion/exclusion criteria and evaluating fecal occult blood test (FOBT). Participants were required to have negative FOBT to ensure bleeding risk was as low as possible prior to randomization. Abnormal FOBT was not an actionable safety measure but excluded a participant from this study. Participants were advised to avoid consumption of red meat (such as beef, lamb, goat, bison, and liver) and more than approximately 250 mg of vitamin C per day for at least 72 hours prior to collection of the first stool sample for FOBT through the last stool sample collection during the treatment period. These dietary restrictions were to avoid misinterpretation of the FOBT results and HemoQuant assessments. Participants were advised to abstain from alcohol for the duration of the study to avoid risk of bleeding due to alcohol-related gastritis. A third clinic visit was completed at day 8 to reconfirm inclusion/exclusion criteria, to evaluate a second FOBT, to administer REGN9933 or REGN7508, and to distribute aspirin and rivaroxaban. The FOBT result on day 8 was reviewed to reconfirm inclusion/exclusion criteria prior to randomization and administration/distribution of study treatments, as a negative FOBT at visits 2 and 3 was required for inclusion in the study. A fourth clinic visit took place on day 29±3 to assess compliance (via pill counts and review of pill diary), adverse events (AEs), and laboratory evaluations (including hematology). Participants were followed until the end of study (EOS) clinic visit on day 100.

The laboratory performing HemoQuant testing was blinded to sample origin. Safety was assessed by monitoring of AEs, with particular attention paid to bleeding events. Further, lab testing of hemoglobin was assessed at screening (visit 1), baseline (visit 2), visit 4, and EOS (visit 5).

### Dose Selection

Participants were randomized to take aspirin or aspirin combined with an anticoagulant (REGN9933, REGN7508, or rivaroxaban). Doses of aspirin and aspirin combined with rivaroxaban were chosen to be clinically relevant, safe, and yet likely to result in increases in subclinical GI bleeding based on available clinical evidence.

### Rationale for Aspirin Dose

Aspirin 75 mg QD was chosen since “low dose” aspirin is in common clinical usage and is recommended by guidelines for use in a variety of patients including those with atherosclerotic cardiovascular disease. In study R0000-HV-2229, aspirin 75 mg once daily (QD) treatment was well tolerated and resulted in a statistically significant increase in FHC over baseline as measured by HemoQuant assay.

### Rationale for Rivaroxaban Dose

The combination of aspirin 75 mg QD and rivaroxaban 20 mg QD is also clinically relevant. Rivaroxaban 20 mg QD is the dose approved in the EU and UK for the indication of prevention of stroke in nonvalvular atrial fibrillation. Notably, in a large (N=14,264) trial of rivaroxaban 20 mg QD in atrial fibrillation, approximately 35% of patients took aspirin at some point during the study (Patel, 2011).

Results from published clinical studies carried out in healthy male participants have demonstrated that rivaroxaban is well tolerated in healthy volunteers at single doses up to 80 mg, or multiple doses of 30 mg twice daily (BID), and in combination with aspirin (as discussed below); these doses were higher than the dose that will be administered in this clinical study.

This clinical study protocol involves treatment with only 20 mg orally QD, in combination with aspirin 75 mg orally QD.

In study R0000-HV-2229, addition of rivaroxaban 20 mg QD to aspirin 75 mg QD was well tolerated and was associated with increases in FHC relative to aspirin alone, as measured by HemoQuant assay.

### Rationale for REGN9933 Dose

A single 300 mg IV dose of REGN9933 was chosen based on data from the single ascending dose study of REGN9933 in healthy volunteers (study R9933-HV-2107) which explored the safety, PK, and PD effects of single IV or SC doses of REGN9933 up to 300 mg. A single dose of 300 mg REGN9933 administered IV was well tolerated and produced maximal inhibition of FXI activity and maximal effects on aPTT (approximately 2.7-fold change from baseline, corresponding to absolute aPTT values of approximately 60 to 80 sec) in all individuals for ≥14 days, without any measurable effect on PT. The duration of the effect was prolonged and near-maximal increases in aPTT were maintained in all participants for ≥21 days.

Exposures (Cₘₐₓ and AUC) from a 300 mg IV dose are at least 60-fold lower than the NOAEL exposures from the 13-week GLP toxicology study in monkeys (R7508-TX-21013).

### Rationale for REGN7508 Dose

A single 250 mg IV dose of REGN7508 was chosen based on data from an ongoing single ascending dose study of REGN7508 in healthy volunteers (study R7508-HV-21102) which explores the safety, PK, and PD effects of single IV or SC doses of REGN7508 up to 250 mg. A single dose of 250 mg administered IV produced maximal increases in aPTT (3.3-fold) for >14 days, without any measurable effect on PT. On average, aPTT values returned to baseline 35 days postdose for the 250 mg dose. Exposures (Cₘₐₓ and AUC) at the selected dose of 250 mg IV are at least 75-fold lower than NOAEL exposure levels based on either AUC or Cₘₐₓ.

### Study Duration and End of Study (EOS)

The duration of the study for a participant was approximately 100 days, excluding the screening period. The EOS was defined as the date the global last participant completes the last in-person or telephone visit, withdraws from the study, or was lost to follow-up (i.e., the study participant could no longer be contacted by the investigator).

### Population

Up to approximately 56 participants were included in each of the 4 arms, for a total of up to approximately 224 participants.

The study population comprised healthy males and females (postmenopausal or permanently surgically sterile and unable to menstruate), 18 to 55 years of age (inclusive), with low risk for bleeding or thrombosis based on medical history and physical and laboratory evaluations. Approximately 10% of the participants enrolled were first-generation Japanese.

### Inclusion Criteria

A participant must have met the following criteria to be eligible for inclusion in the study:
Male or female 18 to 55 years (inclusive) at the screening visit;
For first-generation Japanese participants:
   Must be Japanese, born in Japan, and have both biologic parents and 4 biologic grandparents who are ethnically Japanese and born in Japan;
   Have maintained a Japanese lifestyle, with no significant change since leaving Japan, including having access to Japanese food and adhering to a Japanese diet; and
   Have lived <10 years outside of Japan;
Body mass index between 18.0 and 32.5 kg/m² (inclusive) at the screening visit;
Judged by the investigator to be in good health based on medical history, physical examination, vital sign measurements, and ECGs performed at screening and/or prior to administration of initial dose of study treatment;
Participant was in good health based on laboratory safety testing obtained at the screening visit and/or prior to administration of initial dose of study treatment;
Note: Participants with suspected or confirmed Gilbert's disease could be enrolled in the study;
Normal aPTT, normal PT, and normal platelet counts at screening period and at the day 1 visit as defined by the local laboratory;
Hemoglobin value≥11.0 g/dL for females and ≥12.9 g/dL for males values within the normal range, per local laboratory, at the screening and day 1 visits;
Negative FOBT at Baseline (visit 2) and visit 3;
Note: The FOBT result at visit 3 was reviewed to reconfirm inclusion/exclusion criteria prior to randomization and distribution of study treatments at visit 3. If the FOBT test was positive or any of the inclusion/exclusion criteria were no longer met at visit 3, the participant was not randomized and did not take the study treatments;
Participant was willing and able to comply with clinic visits and study-related procedures; and
Provide informed consent signed by study participant.

### Exclusion Criteria

A participant who met any of the following criteria was excluded from the study:
History of any major surgical procedure or clinically-significant physical trauma in the last 6 months, in the opinion of the investigator, that may pose a risk to the participant by study participation;
Whole blood donation within the previous 56 days or plasma donation within the previous 7 days prior to the screening visit;
Members of the clinical site study team and/or his/her immediate family;
Pregnant or breastfeeding women;
Women of child-bearing potential (WOCBP), defined as women who are fertile following menarche until becoming postmenopausal, unless permanently sterile. Permanent sterilization methods include hysterectomy, bilateral salpingectomy, and bilateral oophorectomy;
A postmenopausal state was defined as no menses for 12 months without an alternative medical cause. A high follicle-stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to the Clinical Trial Facilitation Group guidance;
Sexually active men with WOCBP partners who were unwilling to use the following forms of medically acceptable birth control during the study through day 100 (EOS): vasectomy with medical assessment of surgical success OR consistent use of a condom;
History of clinically significant cardiovascular, respiratory, hepatic, renal, gastrointestinal, endocrine, hematological, psychiatric, neurological, or dermatologic disease, as assessed by the investigator, that may confound the results of the study or poses an additional risk to the participant by study participation. This includes but is not limited to history of thrombotic events, known bleeding disorders, iron-deficiency anemia, central nervous system bleeding, and gastrointestinal bleeding including that caused by hemorrhoids or anal fissures;
Presents any concern to the study investigator that might confound the results of the study or poses an additional risk to the participant by their participation in the study;
Hospitalized for any reason within 30 days of the screening visit;
eGFR (using the Modification of Diet in Renal Disease study equation, the Chronic Kidney Disease Epidemiology Collaboration equation, or equivalent equation) of <60 mL/min/1.73 m² at screening;
Note: If participant had an eGFR below 60 mL/min/1.73 m², one repeat test was allowed. Participant could be enrolled if repeat test demonstrates eGFR of >60 mL/min/1.73 m²;
Current smoker or former smoker, including e-cigarettes, who stopped smoking within 12 months prior to the screening visit;
History of illicit drug or alcohol abuse within the last 2 years prior to the day 1 visit. Note: Urine drug screen could be collected according to the site's operating procedures and/or at the discretion of the investigator;
Was positive for human immunodeficiency virus (HIV) or hepatitis B surface antigen (HBsAg) at the screening visit. Evidence of prior hepatitis B immunization or prior resolved hepatitis B infection was not an exclusion;
Any malignancy, except for nonmelanoma skin cancer or cervical/anus in situ, that had been resected with no evidence of metastatic disease for 3 years prior to the screening visit;
History of significant multiple and/or severe allergies (e.g., latex gloves) or has had an anaphylactic reaction to prescription or nonprescription drugs or food;
Participated in any clinical research study evaluating another investigational drug including biologics or therapy, including specific immunotherapy, within 90 days or at least 5 half-lives (whichever is longer) of an investigational biologic drug or at least 4 weeks for other investigational drug prior to the screening visit;
Use of any prescription and nonprescription medications or nutritional supplements from approximately 2 weeks or 5 half-lives, whichever is longer, prior to first administration of the study treatment through the EOS except the permitted medications listed. This includes anticoagulants and antiplatelets, including NSAIDs or salicylic acid;
Had received a COVID-19 vaccination within 1 week of day 1 or for which the planned COVID-19 vaccinations would not be completed 1 week prior to day 1;
Unwilling or unable to comply with the permitted and prohibited medications and procedures specifications or dietary restrictions for this study; or
Had elective surgery planned to occur prior to EOS.

### Treatments

REGN9933 was provided as a 300 mg IV single dose. REGN7508 was provided as a 250 mg IV single dose. Aspirin was provided as a 75 mg oral dose, once daily, for two weeks. Rivaroxaban was provided as a 20 mg oral dose, once daily, for two weeks.

TABLE 11
Study Design

| Intervention Name | Aspirin | REGN9933 | REGN7508 | Rivaroxaban |
|---|---|---|---|---|
| Intervention | 75 mg | 300 mg | 250 mg | 20 mg |
| Description | Aspirin | REGN9933 | REGN7508 | Rivaroxaban |
| | PO, QD | IV, once | IV, once | PO, QD |
| Type | Drug | Biologic | Biologic | Drug |
| Dose | Tablet | Lyophilized | Lyophilized | Tablet |
| Formulation | | Powder | Powder | |
| Unit Dose | 75 mg | 265 mg in | 265 mg in | 20 mg |
| Strength(s) | | 20 mL vial | 20 mL vial | |
| Dosage Level(s) | 75 mg QD | 300 mg, once | 250 mg, once | 20 mg QD |
| | for 2 weeks | | | for 2 weeks |
| Route of | Oral | IV infusion | IV infusion | Oral |
| Administration | | | | |

### Endpoints

The primary endpoint was the change from baseline in fecal hemoglobin content (FHC) as measured by the average of week 1 and 2 FHC compared with the baseline FHC. The secondary endpoints were incidence and severity of treatment-emergent adverse events (TEAE) through the end of study (EOS); incidence of major bleeding and clinically relevant non-major (CRNM) bleeding (International Society on Thrombosis and Haemostasis [ISTH] criteria) through the EOS; concentrations of REGN9933 through EOS; concentrations of REGN7508 through EOS; change in activated partial thromboplastin (aPTT) time and prothrombin time (PT) from baseline through EOS; incidence and titer of anti-drug antibodies (ADA) to REGN9933 over time; and incidence and titer of ADA to REGN7508 overtime. The exploratory endpoints were the incidence of minor bleeding through the EOS; FHC; differences in baseline-adjusted FHC in each arm in stool samples from week 2 of the treatment period as compared to stool samples from week 1 of the treatment period; absolute concentration over time and change of FXI concentration from baseline; and the change in measures of coagulation pathway function compared to baseline, including, but not limited to FXI functional activity (FXI:C).

### Procedures and Assessments

### Efficacy Procedures

Stool samples for quantitative fecal hemoglobin test (HemoQuant) were collected at home and returned to the study site.

### Safety Assessments

Safety assessments included monitoring of vital signs, physical examination, ECG, laboratory testing, and the monitoring and reporting of AEs.

Blood samples were collected for drug concentration, Factor XI concentration, ADA, aPTT, PT, FXI activity, immunoglobulin G, and exploratory biomarker and research measurements and assessments.

### Results and Conclusions

Methods: In this Phase 1, randomized, open-label study (NCT06444178), healthy volunteers received single-dose intravenous Factor XI inhibitor REGN9933 300 mg or REGN7508 250 mg+aspirin 75 mg once daily (QD); rivaroxaban 20 mg QD+aspirin 75 mg QD; or aspirin 75 mg QD alone. Primary endpoint was fecal hemoglobin change assessed by HemoQuant stool test. Pooled REGN9933/REGN7508+aspirin data were compared with rivaroxaban+aspirin post hoc. Secondary endpoints included impact of REGN9933/REGN7508 on activated partial thromboplastin time (aPTT) and prothrombin time (PT).

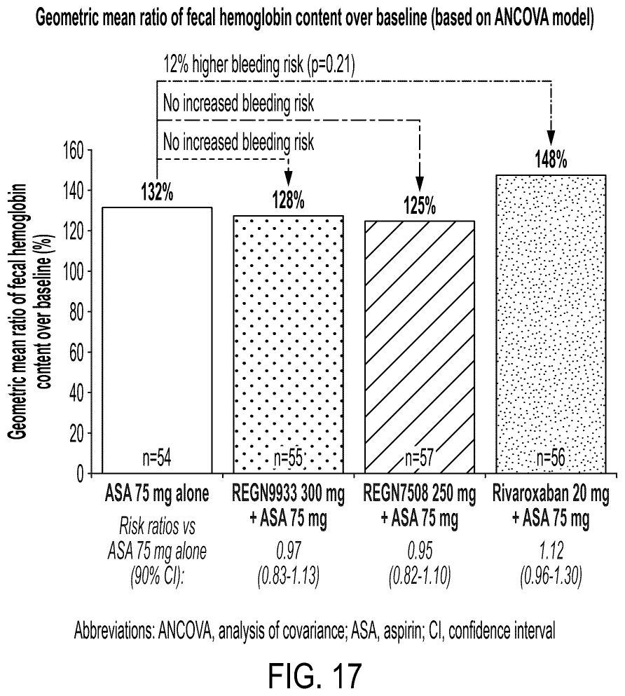

Overall, 224 participants were treated. REGN9933+aspirin and REGN7508+aspirin caused no more bleeding vs aspirin alone, whereas rivaroxaban+aspirin increased bleeding by 12% vs aspirin alone (FIG. 17). Pooled REGN9933/REGN7508+aspirin data showed 14% less bleeding vs rivaroxaban+aspirin (p<0.05). REGN9933 and REGN7508 showed durable aPTT prolongation without impacting PT.

Addition of Factor XI inhibitors did not increase subclinical GI bleeding vs aspirin alone, whereas rivaroxaban+aspirin increased bleeding vs aspirin alone.

This Phase 1 study demonstrated that rivaroxaban plus ASA increased subclinical GI bleeding more than ASA alone, whereas the addition of Factor XI inhibitors did not increase GI bleeding in healthy volunteers. Focused Factor XI inhibition with REGN9933 or REGN7508 may address the unmet need for an alternative adjunctive antithrombotic approach on top of antiplatelet therapies, with no increased risk of bleeding compared with standard-of-care anticoagulants.

### Example 8: A Multicenter, Randomized, Open-Label, Active Control Study of REGN7508 for Prevention of Venous Thromboembolism After Elective, Unilateral, Total Knee Arthroplasty

The population for this study was adults undergoing elective unilateral total knee arthroplasty (TKA). Participants less than 50 years of age were excluded, due to a significantly lower incidence of symptomatic venous thromboembolism (VTE) relative to older participants (Lee, 2015). Weight greater than 130 kg was exclusionary to limit the variability in exposure and PD effect of the fixed dosing regimen of the antibody. Since venography provided most of the events that make up the primary endpoint, those with or at high risk of contrast allergy were excluded. Participants with contraindications to enoxaparin, such as a history of HIT were not enrolled. Several risk factors for abnormal bleeding or clotting were also exclusionary. Finally, WOCBP were excluded, since the risk of bleeding in the setting of FXI inhibition in this subpopulation is not yet characterized; further, the age cutoff of >50 years of age already precludes many WOCBP.

Participants were stratified by 2 factors: age (<70 vs ≥70 years of age), based on the higher incidence of DVT in the ≥70 years age group (Lee, 2015); and site, to account for local variation in surgical and anesthesia practices.

Several medications are approved in the countries in which the trial is being conducted for the prevention of VTE after TKA; therefore, a placebo-treated group would not have been appropriate. Enoxaparin, a LMWH, is approved for the prevention of VTE after orthopedic surgery. In EU countries, the approved dose is 40 mg SC once daily (Inhixa [Summary of Product Characteristics], 2021) (CLEXANE [SmPC], 2022). Dosing started 12 to 24 hours after surgery in this study, per the label. The duration of treatment was a minimum of 7 to 14 days per the label, and this could be extended up to 35 days (Inhixa [Summary of Product Characteristics], 2021) (CLEXANE [SmPC], 2022) although the benefit of longer duration treatment is more pronounced after total hip replacement relative to TKA (Comp, 2001) (Eikelboom, 2001). In this study, study drug treatment occured through the time of venography (or day 12, whichever was earlier). Several recent studies using this paradigm have utilized enoxaparin as an active comparator (Büller, 2015) (Verhamme, 2021) (Weitz, 2020) (Weitz, 2021), allowing for benchmarking of results from this study to other agents in development.

Timing of initiation of anticoagulants for prophylaxis of thrombosis following total knee replacement is a balance between safety and efficacy. Dosing closer to the time of surgery may confer additional efficacy but at the expense of increased bleeding risk. Enoxaparin was started at 12 to 24 hours post-operatively, consistent with labeled dosing regimens and REGN7508 was also dosed at 12 to 24 hours post-surgery, to allow a direct comparison. Dosing of FXI inhibitors in this time range, as well as pre-operatively, has been shown to be well-tolerated in published trials of FXI inhibitors (Büller, 2015) (Verhamme, 2021) (Weitz, 2020).

Regarding duration of treatment, for participants assigned to study drug treatment with enoxaparin, subsequent VTE prophylaxis (beyond day 12 or day of venography) was determined by the site investigator as guided by local standard of care. However, for participants assigned to REGN7508, use of anticoagulants for VTE prophylaxis was prohibited, because REGN7508 is expected to elevate aPTT for up to 28 days, returning to baseline by 35 days postdose.

Treatment is administered in an open-label manner because enoxaparin is packaged in pre-filled syringes, making blinding operationally challenging. In addition, performing the study in a blinded manner would have required some (one third) of the participants to take placebo injections, which would have increased participant burden. To mitigate bias, participants were randomized to treatment assignment. To further mitigate bias in outcome assessment, adjudication of outcomes related to VTE and bleeding (i.e., results of venograms and any confirmatory study for symptomatic VTE; categorization of bleeding events) were performed by a centralized independent AC that is blinded to treatment assignment.

The primary efficacy endpoint, which is a composite of confirmed asymptomatic DVT, confirmed symptomatic VTE (symptomatic DVT of the leg or nonfatal PE), and unexplained death for which PE cannot be ruled out, was designed to comprehensively capture VTE events and has been used in prior post-TKA VTE studies (Verhamme, 2021) (Weitz, 2020) (Weitz, 2021). Asymptomatic DVT is best detected with venography because of its high sensitivity relative to other modalities. In a study that directly compared venography to ultrasound for detection of asymptomatic DVT following hip or knee replacement surgery in patients receiving anticoagulant prophylaxis, the overall incidence of DVT detected by venography was 18.9%, compared to 11.5% with ultrasound (Schellong, 2007). Unilateral venography of the operated leg detects more than 80% of DVTs in patients undergoing unilateral TKA. Relative to bilateral procedure, unilateral venography reduces discomfort and improves compliance with the procedure with relatively limited loss of event detection. Venography is performed at day 10 (±2 days), based on early studies suggesting that most DVTs form prior to day 12 after surgery (Gallus, 1976).

The safety secondary endpoint was chosen to capture clinically relevant bleeding events. The ISTH criteria will be used for both major (Schulman, 2010) and CRNM (Kaatz, 2015) bleeds. Both have clear, consensus definitions that allowed for robust adjudication.

Minor bleeds were not included in the secondary safety endpoint due to lack of clear criteria, which are crucial due to unblinded assignment of treatment. As minor bleeding occurs more frequently and might therefore be of interest, incidence of minor bleeding was tabulated separately as an exploratory endpoint. Minor bleeding comprises any bleeding AE that does not qualify as either major bleeding or CRNM.

The length of study follow-up was 75 days. Data from the concurrent FIH study, R7508-HV-21102, showed that aPTT had returned to within 20% of normal values in all treated participants by day 36 following a single 250 mg IV dose, which is the dose used in this trial. Enoxaparin will be dosed through the time of venography (or day 12, whichever is earlier); based on the half-life (4.5 to 7 hours), pharmacologically relevant levels are not present a few days after the last dose.

### Objectives

The primary objective of this study is to evaluate the efficacy of REGN7508 for the prevention of venous thromboembolism (VTE) after unilateral total knee arthroplasty (TKA), compared to enoxaparin. The secondary objectives were to evaluate the bleeding risk (i.e. major and CRNM bleeding) of REGN7508 after unilateral TKA through time of venography, compared to enoxaparin; assess overall safety and tolerability of REGN7508 in participants undergoing TKA; evaluate the efficacy of REGN7508 in prevention of clinically-relevant VTE, compared to enoxaparin; evaluate the efficacy of REGN7508 in prevention of DVT detected by venography, compared to enoxaparin; evaluate the PK of REGN7508 after single IV administration; assess PD effects of REGN7508 on intrinsic and extrinsic coagulation pathways (i.e., activated partial thromboplastin time [aPTT], prothrombin time [PT]); and assess immunogenicity following a single dose of REGN7508 over time. The exploratory objectives were to explore the efficacy of REGN7508 in prevention ofVTE after unilateral TKA through the EOS, compared to enoxaparin; explore the efficacy of REGN7508 in prevention of symptomatic, clinical thrombosis, compared to enoxaparin; explore the extent of DVT burden on venography in participants receiving REGN7508 compared to enoxaparin; explore the bleeding risk (i.e., major bleeds and CRNM) of REGN7508 after unilateral TKA through EOS compared to enoxaparin; explore the occurrence of minor bleeding compared to enoxaparin; explore biomarkers related to FXI inhibition by REGN7508; expand molecular understanding of venous thromboembolism, and related diseases; explore relationships between PK, biomarkers of anticoagulant activity, and efficacy; study REGN7508 mechanism of action in the coagulation cascade, VTE, and related disease; explore whether potential differences in participant efficacy and safety are associated with genotype and gene expression and further study FXI and coagulation-related diseases, using optional whole blood collected from consented participants.

### Study Design

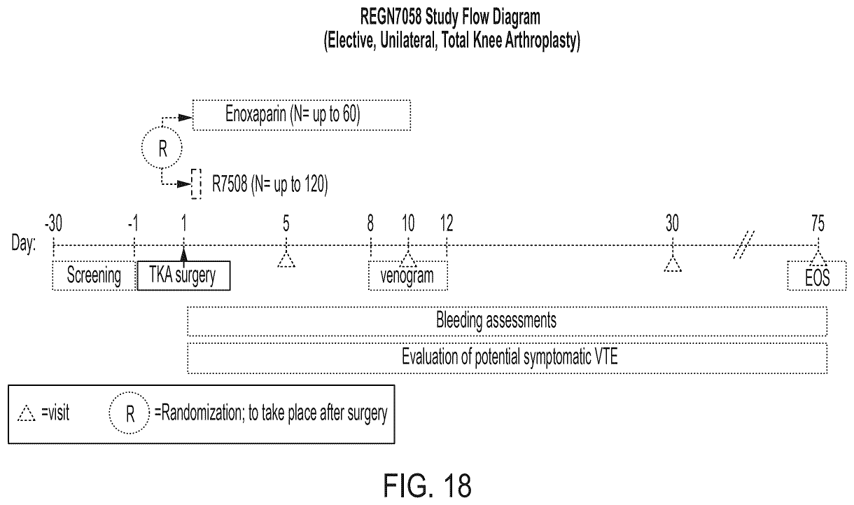

This is a multicenter, multinational, randomized, open-label, active control study of a single dose of IV REGN7508 for prevention of VTE in participants undergoing an elective unilateral TKA (FIG. 18).

Participants who were at least 50 years of age and undergoing elective unilateral TKA were randomized after surgery to 1 of 2 treatment arms—REGN7508 or enoxaparin—in a ratio of 2:1, in a parallel manner. Participants will be stratified based on study site and age (<70 vs ≥70 years of age). Approximately 120 participants were enrolled in the REGN7508 arm, and 60 participants were enrolled in the comparator arm, for a total of up to approximately 180 participants in the study.

Participants were randomized after completion of surgery to receive a single dose of REGN7508 250 mg IV, or enoxaparin 40 mg subcutaneous (SC) daily through the time of venography.

Discharge from the hospital was determined by the site investigator based on individual participant recovery and local practice. Participants underwent unilateral venography of the operated leg performed on day 10 (+2 days). While in the hospital after surgery and at subsequent study visits, participants were assessed for signs and symptoms of symptomatic DVT and pulmonary embolism (PE). If DVT or PE was suspected, objective confirmatory testing was obtained using either lower extremity ultrasound, conventional venography, computed tomography venography, or magnetic resonance venography.

Participants were followed until the end-of-study (EOS) visit on day 75. Assessments included scheduled venography; adverse event (AE) monitoring; physical exams; clinical laboratory testing (hematology, chemistry, coagulation, and urinalysis); electrocardiogram (ECG); PK, PD, and biomarker sampling.

### Dose Selection

A single 250 mg IV dose of REGN7508 was selected based on data from the ongoing first-in-human (FIH) study, a single ascending dose study with REGN7508 in healthy volunteers (see Example 5 above), which explored the safety, PK, and PD effects, of single IV doses of REGN7508 up to 250 mg. Study participants each received a single 0.5 hour IV infusion of REGN7508 or matching placebo and blood samples for measurement of concentrations of REGN7508 in serum and PD assays (including Factor XI activity, PT and aPTT) were collected at intervals. REGN7508 exposure (AUC and Cₘₐₓ) increased proportionally to dose between 5 and 125 mg. Based on limited data (concentration on day 8 only), exposure appeared to increase proportionally with the 250 mg dose. REGN7508 treatment had no apparent effect on PT, but produced a rapid, dose-dependent decrease in Factor XI activity and a rapid, dose-dependent increase in aPTT without significant safety, tolerability, or bleeding findings.

Single doses of REGN7508≥15 mg produced a dose-dependent sustained inhibition of FXI activity and increases in aPTT, with a mean maximum fold increase of 3.3, which was achieved at the 30 mg dose. Further increase in dose did not result in an increase in the magnitude of aPTT prolongation, but in longer duration of the effect. Based on these observed data it is expected that a REGN7508 dose of 250 mg will maintain maximal inhibition of FXI activity and maximal effects on aPTT in all individuals for ≥14 days. PD data from study Example 5, above, showed that aPTT values returned to within 20% of normal values by 35 days following the 250 mg dose.

REGN7508 exposures from a 250 mg IV dose are projected to be approximately >60-fold lower than corresponding exposures at the non-clinical toxicology NOAEL.

### Study Duration and End of Study (EOS)

The duration of the study for a participant was approximately 75 days, excluding the screening period. The definition for EOS (trial) is the date of global last participant's last visit, or the date of withdrawal from the study, or lost to follow-up (i.e., the study participant can no longer be contacted by the investigator).

### Population

The study population comprised adults who were at least 50 years of age and undergoing a primary elective, unilateral TKA.

### Inclusion Criteria

A participant must have met the following criteria to be eligible for inclusion in the study:
Is a male or female≥50 years of age at the screening visit, undergoing a primary elective unilateral TKA;
Has a body weight≤130 kg at screening visit;
Is judged by the investigator to be in good health based on medical history, physical examination, vital sign measurements, and ECG's performed at screening and/or prior to administration of initial dose of study drug;
Is in good health based on laboratory safety testing obtained during the screening period, including: a. Platelet count>150K; b. Normal INR and aPTT during screening; c. Hemoglobin>10/dL; d. ALT or AST<3×ULN, or total bilirubin<2× ULN;
Note: Participants with suspected or confirmed Gilbert's disease can be enrolled in the study;
Note: Laboratory assessments for eligibility may be repeated once at the principal investigator's discretion. The participant can be enrolled if the repeat test demonstrates a laboratory value that meets eligibility criteria;
Willing and able to comply with clinic visits and study-related procedures including venography; and
Provide informed consent signed by the study participant.

### Exclusion Criteria

A participant who met any of the following criteria was excluded from the study:
Any condition that, as assessed by the investigator, may confound the results of the study or poses an additional risk to the participant by study participation;
History of bleeding in the 6 months prior to dosing requiring hospitalization or transfusion;
history of intracranial or intraocular bleeding, excessive operative or post-operative bleeding, and traumatic spinal or epidural anesthesia; history of bleeding diathesis (e.g., Hemophilia A or B, von Willebrand's Factor deficiency);
History of thromboembolic disease or thrombophilia;
History of major surgery, including brain, spinal, or ocular, within approximately the past 6 months;
History of major trauma within approximately the past 6 months prior to dosing;
Hospitalized (>24 hours) for any reason within 30 days of the screening visit;
Using the MDRD equation, has an estimated GFR of <45 mL/min/1.73 m² at the screening visit;
Note: If participant has a GFR below 45 mL/min/1.73 m², 1 repeat test is allowed. Participant may be enrolled if repeat test demonstrates GFR of >45 mL/min/1.73 m²;
History of hypersensitivity or contraindication (per local label) to any of the study treatments or their excipients;
Contraindication to anticoagulation in the opinion of the investigator;
Has received or plans to receive preoperative enoxaparin on the day prior to TKA surgery;
Allergy to contrast agents, poor venous access, or other factors that might preclude venography;
Expected post-operative use of epidural/spinal catheter;
Participants who have traumatic epidural/spinal anesthesia or excessive intra-operative blood loss during TKA surgery should not be randomized to receive study treatment;
Has a history of alcohol or drug abuse;
Note: Urine drug screen may be collected according to the site's operating procedures and/or at the discretion of the investigator;
Known history of HIV infection, or ongoing chronic hepatitis B or hepatitis C infection;
Prior resolved hepatitis B infection is not an exclusion;
Any malignancy, except for nonmelanoma skin cancer or cervical/anus in situ, that have been resected with no evidence of metastatic disease for 3 years prior to the screening visit;
History of significant multiple and/or severe allergies (e.g., latex gloves) or has had an anaphylactic reaction to prescription or nonprescription drugs or food;
Participated in any clinical research study evaluating another investigational drug including biologics or therapy, including specific immunotherapy, within 90 days or at least 5 half-lives (whichever is longer) of an investigational biologic drug or at least 4 weeks for other investigational drug prior to the screening visit;
Unwilling or unable to comply with the prohibited medication specifications for this study, including: a. Participants who require therapeutic (full-dose) anticoagulation should be excluded; b. Participants who require antiplatelet therapy (except for Acetylsalicylic acid [ASA]≤100 mg/day) should be excluded;
Presents any concern to the study investigator that might confound the results of the study or poses an additional risk to the participant by their participation in the study;
Members of the clinical site study team and/or his/her immediate family, unless prior approval granted by the sponsor;
Has received a COVID-19 vaccination within 1 week of planned start of study medication or for which the planned COVID-19 vaccinations would not be completed 1 week prior to start of study drug;
Sexually active men with WOCBP partners who are unwilling to use medically acceptable birth control through day 75 (EOS): vasectomy with medical assessment of surgical success OR consistent use of a condom.
Women of childbearing potential, defined as women who are fertile following menarche until becoming postmenopausal, unless permanently sterile. Permanent sterilization methods include hysterectomy, bilateral salpingectomy, and bilateral oophorectomy; or
A postmenopausal state is defined as no menses for 12 months without an alternative medical cause. A high follicle-stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to the Clinical Trial Facilitation Group guidance. Pregnancy testing and contraception are not required for women who are postmenopausal or permanently sterile.

### Treatments

REGN7508 was provided as a 250 mg IV single dose. Enoxaparin was provided as a 40 SC dose, once daily, through the time of venography (or day 12, whichever is earlier).

TABLE 12
Study Design

| Intervention Name | REGN7508 | Enoxaparin |
|---|---|---|
| Intervention Description | 250 mg IV, | 40 mg SC, daily through time of venography |
| | single dose | (or day 12, whichever is earlier) |
| Dose Formulation | Lyophilized | Pre-filled Syringe-Liquid Solution |
| | Powder | |
| Unit Dose Strength(s) | 265 mg in 20 | 40 mg, 0.4 mL, pre-filled syringe |
| | mL vial | |
| Dosage Level(s) | 250 mg, once | 40 mg, daily, through the time of venography |
| | | (or day 12, whichever is earlier) |
| Route of Administration | IV infusion | SC injection |

### Endpoints

The primary endpoint is the incidence of confirmed, adjudicated VTE through day 12 (REGN7508 vs enoxaparin). The secondary endpoints are: incidence of major bleeding and CRNM bleeding through time of venography (or day 12, whichever is earlier); incidence of treatment-emergent adverse events (TEAE) through the EOS; incidence of major VTE through day 12 (REGN7508 vs enoxaparin); incidence of DVT as measured by venography of the operated leg on day 10±2 days (REGN7508 vs enoxaparin); concentrations of REGN7508 through EOS; change in aPTT from baseline through end of study; change in PT from baseline through EOS; and incidence and titer of anti-drug antibodies (ADA) to REGN7508 through end of study. The exploratory endpoints are: incidence of major VTE through the EOS; incidence of confirmed symptomatic DVT in either leg through day 12 and through the EOS; incidence of confirmed pulmonary embolism (PE) through day 12 and through the EOS; incidence of fatal PE, which includes sudden death for which PE cannot be ruled out, through day 12 and through the EOS; in participants with DVT detected on venogram, distribution of DVT (proximal versus distal), for proximal DVT, size of VTE, for distal DVT, the number of veins affected; incidence of major bleeding and CRNM bleeding through EOS; incidence of minimal bleeding through time of venography and through the EOS; and change in measures of coagulation pathway function compared to baseline including, but not limited to: FXI:C (FXI coagulant activity assay), FXI concentration, and TGA for extrinsic and intrinsic pathways.

### Procedures and Assessments

Efficacy Procedures: Venography of the operated leg was performed on day 10 (+2 days). If DVT or PE was suspected at any point during the study, objective confirmatory testing was carried out.

Safety Procedures: Safety assessments included monitoring of vital signs, physical examination, ECG, assessment for bleeding, laboratory testing, and the monitoring and reporting of AEs.

Blood samples were collected for drug concentration, Factor XI concentration, ADA, and exploratory biomarker and research measurements and assessments.

### Statistical Plan

Statistical Hypothesis: The clinical hypothesis comparing the efficacy of REGN7508 to enoxaparin was assessed through a Bayesian estimation framework. Bayesian posterior probabilities were reported to estimate the incidence of confirmed VTE for participants treated with REGN7508 and enoxaparin, as well as the odds ratio (OR) of confirmed VTE in participants administered REGN7508 as compared to that of enoxaparin (OR_{RE}). The statistical hypothesis was OR_{RE}<1, i.e., the incidence of confirmed VTE was lower in the REGN7508 arm compared to the enoxaparin arm.

Justification of Sample Size: Up to a total of approximately 180 participants were randomized in a 2:1 allocation ratio to REGN7508 and enoxaparin.

The sample size was determined through Bayesian approach. The prior distribution of the treatment difference was assumed to be log(OR)~N(0,0.843), which was expressed as the log(OR). The prior distribution follows a normal distribution centered around no treatment difference and with a variance such that extreme treatment differences are expected to be unlikely, i.e., the probability that OR is between 0.25 and 4 is 90%.

For the purpose of choosing a sample size, the following quantities were estimated through simulation under the assumed event rates of VTE (13% for REGN7508 vs 22.4% for enoxaparin):
The probability of any beneficial effect of REGN7508 over enoxaparin, i.e., the posterior distribution of the OR comparing REGN7508 to enoxaparin of confirmed VTE. A beneficial effect is where OR_{RE}<1, and thus the study will estimate P(OR_{RE}<1|data).
In 75% of the simulated trials, in studies where the P(OR_{RE}<1|data)>0.95, the sample size was 120 participants or less. Therefore, a sample size of 120 participants per group was chosen.

For the active control arm (enoxaparin), the data from the previous trial (ROXI-VTE I) was incorporated through a Bayesian frame using the power prior approach. The weight of the prior data could be adjusted using a power parameter, which scales from 0 to 1, according to the similarity of the prior and current trial data. The power parameter was chosen to be 0.5, which is equivalent to discounting half of the prior data from the control arm in ROXI-VTE I. As a result, the active control (enoxaparin) arm in the present study was reduced by half compared to the size of the enoxaparin arm in ROXI-VTE I and hence 60 participants were enrolled in the enoxaparin arm in this study.

Statistical Methods: The treatment effect was estimated using the posterior distribution of OR_{RE}. For comparison of REGN7508 vs enoxaparin, different posterior probabilities will be provided, such as P(OR<0.5, 0.9, 1.0, 1.1, 1.2 etc|data).

The entire posterior density function for the treatment effect (OR_{RE}) was computed, along with the cumulative posterior distribution, which quantifies evidence for all possible treatment effects. A prior for treatment difference log(OR)~N(0,0.843) will be used. Bayesian summary statistics including two-sided 95% Highest Density Interval, posterior mean, and median were reported. Summary statistics based on the raw data including the number of participants reflected in the calculation (n), mean, standard deviation, Q1, median, Q3, minimum, and maximum were reported.

Safety was assessed by relevant parameters, including vital signs, physical examinations, ECGs, laboratory evaluations, and AEs.

### Results

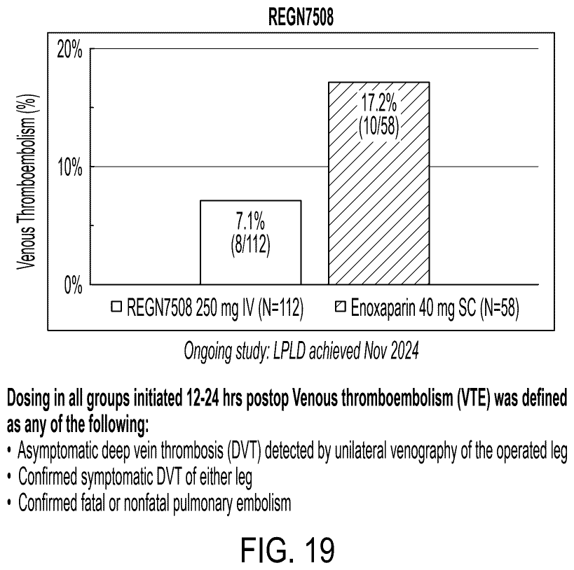

After administration of 250 mg IV REGN7508 12-24 hours post-surgery, the rate of VTE in the study population was 7.1%, see Table 13 and FIG. 19. The previously-published event rate for a placebo group was ~60%.

TABLE 13
Results

| Therapy | Dose | VTE Rate |
|---|---|---|
| Placebo | N/A | ~60% |
| REGN7508 | 250 mg IV | 7.1% |
| REGN9933 | 300 mg IV | 17.2% |

On the measure of venous thromboembolism (VTE) rates following surgery, a pooled analysis across both trials (Examples 8 and 9) showed REGN7508 was superior to enoxaparin and noninferior to apixaban, and REGN9933 was non-inferior to enoxaparin. All VTE events were asymptomatic, except for one symptomatic case of pulmonary embolism in the apixaban arm. Additionally, no major bleeding (including surgical site bleeding) or clinically relevant non-major bleeding occurred in any study arm. The only treatment-related AE in any arm was one case of minimal bleeding (contusion) reported in the enoxaparin arm. There were no treatment-related serious AEs (SAEs) in any arm.

### Example 9: A Multicenter, Randomized, Open-Label, Active-Control Study of REGN9933 For Prevention of Venous Thromboembolism After Elective, Unilateral, Total Knee Arthroplasty

The population for this study was adults undergoing elective, unilateral total knee arthroplasty (TKA). Participants less than 50 years of age were excluded, due to a significantly lower incidence of symptomatic venous thromboembolism (VTE) relative to older participants (Lee, 2015). Weight greater than 130 kg was exclusionary to limit the variability in exposure and PD effect of the fixed dosing regimen of the antibody. Since venography provided most of the events that make up the primary endpoint, those with or at high risk of contrast allergy were excluded. Participants with contraindications to enoxaparin, such as a history of heparin-induced thrombocytopenia (HIT), or apixaban were not enrolled. Several risk factors for abnormal bleeding or clotting were also exclusionary. Finally, women of child-bearing potential (WOCBP) were excluded, since the risk of bleeding in the setting of FXI inhibition in this subpopulation is not yet characterized; further, the age cutoff of >50 years of age already precludes nearly all WOCBP.

Participants was stratified by two factors: age (<70 vs ≥70 years of age), based on the higher incidence of DVT in the ≥70 years age group (Lee, 2015); and site, to account for local variation in surgical and anesthesia practices.

Several medications are approved in the countries in which the trial will be conducted for the prevention of VTE after TKA; therefore, a placebo-treated group would not have been appropriate. Enoxaparin, a low molecular weight heparin (LMWH), is approved for the prevention of VTE after orthopedic surgery. In European Union (EU) countries, the approved dose is 40 mg subcutaneous (SC) once daily (Inhixa [Summary of Product Characteristics], 2021). Dosing started 12 to 24 hours after surgery in this study, per the label. The duration of treatment is a minimum of 7 to 14 days per the label, and this could be extended up to 35 days (Inhixa [Summary of Product Characteristics], 2021) (Lovenox [Product Monograph], 2021), although the benefit of longer duration treatment is more pronounced after total hip replacement relative to TKA (Comp, 2001) (Eikelboom, 2001). In this study, treatment occurred through the time of venography (or day 12, whichever is earlier); subsequent VTE prophylaxis could be determined by the site investigator. Several studies using this paradigm utilized enoxaparin as an active comparator (Buller, 2015) (Verhamme, 2021) (Weitz, 2020) (Weitz, 2021), allowing for benchmarking of results from this study to other agents in development.

Apixaban, a DOAC, is approved for the prevention of VTE after orthopedic surgery (Eliquis [Summary of Product Characteristics], 2021), and has also shown efficacy in prevention of VTE after TKA. In these studies, the regimen of apixaban evaluated was 2.5 mg twice daily (BID), initiated 12 to 24 hours after surgery, and continued for 10 to 14 days after surgery (Lassen, 2010) (Lassen, 2009). Apixaban serves as a calibrator in this study; a difference between apixaban and enoxaparin is expected and was descriptively evaluated as a positive control. In addition, it was useful to gain an early understanding of how REGN9933 compares to apixaban.

Timing of initiation of anticoagulants for prophylaxis of thrombosis following total knee replacement is a balance between safety and efficacy. Dosing closer to the time of surgery may confer additional efficacy but at the expense of increased bleeding risk. Apixaban and enoxaparin were started at 12 to 24 hours post-operatively, consistent with labeled dosing regimens and REGN9933 was also dosed at 12 to 24 hours post-surgery, to allow a direct comparison. Dosing of FXI inhibitors in this time range, as well as pre-operatively, has been shown to well-tolerated in published trials of FXI inhibitors (Buller, 2015) (Verhamme, 2021) (Weitz, 2020).

Treatment was administered in an open-label manner because enoxaparin is packaged in pre-filled syringes, making blinding operationally challenging. In addition, performing the study in a blinded manner would have required most (two thirds) of the participants to take placebo injections, which would have increased participant burden. To mitigate bias, participants were randomized to treatment assignment. To further mitigate bias in outcome assessment, adjudication of outcomes related to VTE and bleeding (i.e., results of venograms and any confirmatory study for symptomatic VTE; categorization of bleeding events) was performed by a centralized adjudication committee (AC) that was blinded to treatment assignment.

The primary efficacy endpoint, which is a composite of confirmed asymptomatic DVT, confirmed symptomatic VTE (symptomatic DVT of the leg or non-fatal pulmonary embolism [PE]), and unexplained death for which PE cannot be ruled out, was designed to comprehensively capture VTE events and has been used in prior post-TKA VTE studies (Verhamme, 2021) (Weitz, 2020) (Weitz, 2021). Asymptomatic DVT is best detected with venography because of its high sensitivity relative to other modalities. In a study that directly compared venography to ultrasound for detection of asymptomatic DVT following hip or knee replacement surgery in patients receiving anticoagulant prophylaxis, the overall incidence of DVT detected by venography was 18.9%, compared to 11.5% with ultrasound (Schellong, 2007). Unilateral venography of the operated leg detects more than 80% of DVTs in patients undergoing unilateral TKA. Relative to bilateral procedure, unilateral venography reduces discomfort and improves compliance with the procedure with relatively limited loss of event detection. Venography was performed at day 10 (±2 days), based on early studies suggesting that most DVTs form prior to day 12 after surgery (Gallus, 1976).

The safety secondary endpoint was chosen to capture clinically relevant bleeding events. The International Society on Thrombosis and Hemostasis (ISTH) criteria were used for both major (Schulman, 2010) and CRNM (Kaatz, 2015) bleeds. Both have clear, consensus definitions that allowed for robust adjudication.

Minimal bleeds were not included in the secondary safety endpoint due to lack of clear criteria, which are crucial due to unblinded assignment of treatment. As minimal bleeding occurs more frequently and might therefore be of interest, incidence of minimal bleeding is tabulated separately as an exploratory endpoint. Minimal bleeding comprises any bleeding AE that does not qualify as either major bleeding or CRNM.

The length of study follow-up was 75 days. Data from study R9933-HV-2107 showed that aPTT had returned within 10% of normal values by Day 64 following a single 300 mg IV dose, which was the dose used in this trial. Both enoxaparin and apixaban were dosed through the time of venography (or day 12, whichever is earlier); based on their half-lives (enoxaparin 4.5 to 7 hours, apixaban 12 hours), pharmacologically relevant levels are not present a few days after the last dose.

### Objectives

The primary objective of the study is to evaluate the efficacy of REGN9933 for the prevention of VTE after unilateral TKA, compared to enoxaparin. The secondary objectives of the study are to evaluate the bleeding risk (i.e., major and CRNM bleeding) of REGN9933 after unilateral TKA through time of venography, compared to enoxaparin; to assess overall safety and tolerability of REGN9933 in participants undergoing TKA; to evaluate the efficacy of REGN9933 in prevention of clinically relevant VTE compared to enoxaparin; to evaluate the efficacy of REGN9933 in prevention of DVT detected by venography, compared to enoxaparin; to evaluate the pharmacokinetics (PK) of REGN9933 after single IV administration; to assess PD effects of REGN9933 on intrinsic and extrinsic coagulation pathways (i.e., aPTT, PT); to assess immunogenicity following a single dose of REGN9933 over time; and to compare the efficacy of enoxaparin and apixaban in prevention of VTE after unilateral TKA. The exploratory objectives of the study are to explore the efficacy of REGN9933 in prevention of VTE after unilateral TKA through the end of study, compared to enoxaparin and apixaban; explore the efficacy of REGN9933 in prevention of symptomatic, clinical thrombosis, compared to enoxaparin and apixaban; explore the efficacy of REGN9933 in prevention of DVT detected by venography, compared to apixaban; explore the extent of DVT burden on venography in participants receiving REGN9933 compared to enoxaparin and apixaban; explore the bleeding risk (i.e., major bleeds and CRNM) of REGN9933 after unilateral TKA through end of study compared to enoxaparin and apixaban; explore the occurrence of minor bleeding compared to enoxaparin and apixaban Explore biomarkers related to FXI inhibition by REGN9933; develop a molecular understanding of venous thromboembolism, and related diseases; explore relationships between PK, biomarkers of anticoagulant activity, and efficacy; to study REGN9933 mechanism of action the coagulation cascade, VTE, and related diseases; and explore whether potential differences in participant efficacy and safety are associated with genotype and gene expression and further study FXI and coagulation-related diseases, using optional whole blood DNA and RNA collected from consented subjects.

### Study Design

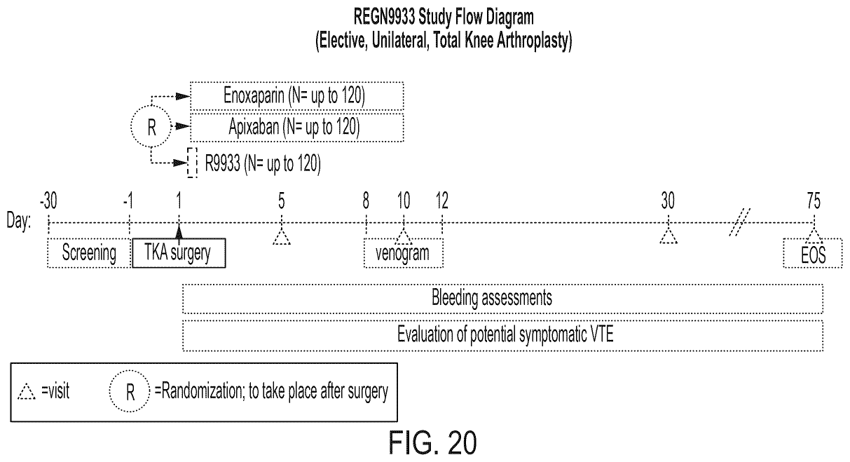

This is a multicenter, multinational, randomized, open label, active control study of a single dose of IV REGN9933 for prevention of VTE in participants undergoing an elective, unilateral, TKA (FIG. 20).

Participants who were at least 50 years of age and undergoing elective, unilateral TKA were randomized after surgery to one of 3 treatment arms-REGN9933, enoxaparin, or apixaban—in a ratio of 1:1:1, in a parallel manner. Participants were stratified based on study site and age (<70 vs ≥70 years of age). Approximately 120 participants will be enrolled in each arm, for a total of up to approximately 360 participants in the study.

Participants were randomized after completion of surgery to receive a single dose of REGN9933 300 mg IV, enoxaparin 40 mg subcutaneous (SC) daily through the time of venography, or apixaban 2.5 mg orally twice daily through the time of venography.

Discharge from the hospital was determined by the site investigator based on individual participant recovery and local practice. Participants underwent unilateral venography of the operated leg performed on day 10 (+2 days). While in the hospital after surgery and at subsequent study visits, participants were assessed for signs and symptoms of symptomatic DVT and pulmonary embolism (PE). If DVT or PE was suspected, objective confirmatory testing was obtained.

Participants were followed until the end-of-study visit on day 75. Assessments included scheduled venography; adverse event (AE) monitoring; physical exams; clinical laboratory testing (hematology, chemistry, coagulation, and urinalysis); electrocardiogram (ECG); PK, PD, and biomarker sampling.

### Dose Selection

A single 300 mg intravenous (IV) dose of REGN9933 was chosen based on data from a single ascending dose study with REGN9933 in healthy volunteers (Example 6, above—study R9933-HV-2107) which explored the safety, PK, and PD effects, of single IV doses of REGN9933 up to 300 mg. Study participants each received a single 0.5 hour IV infusion of REGN9933 or matching placebo and blood samples for measurement of concentrations of REGN9933 in serum and PD assays (including Factor XI activity, PT and aPTT) were collected at intervals. REGN9933 pharmacokinetics were non-linear across the 3 to 300 mg dose range in a manner consistent with target-mediated drug disposition. REGN9933 treatment had no apparent effect on PT, but produced a rapid, dose-dependent decrease in Factor XI activity and a rapid, dose-dependent increase in aPTT without significant safety, tolerability, or bleeding findings.

Single doses of REGN9933≥100 mg produced sustained inhibition of FXI activity and increases in aPTT in all treated individuals. Based on these observed data it is expected that a REGN9933 dose of 300 mg will maintain maximal inhibition of FXI activity and maximal effects on aPTT (~2.8-fold change from baseline, corresponding to absolute aPTT values of approximately 60 to 80 sec) in all individuals for ≥14 days. Pharmacodynamic data from Example 6, above, showed that aPTT values had returned to within 10% of normal values 64 days following the 300 mg dose.

REGN9933 exposures from a 300 mg IV dose are substantially lower than corresponding exposures at the non-clinical toxicology no observed adverse effect level (NOAEL). Observed average total REGN9933 exposures from a 300 mg dose in Example 6, above, were >60× lower than average exposures at the NOAEL dose (100 mg/kg IV) in the 5-week REGN9933 GLP monkey toxicology study (R9933-TX-20070).

Previous studies investigating the use of FXI inhibitors for prevention of post-operative VTE have shown that efficacy correlates with the magnitude of treatment effects on aPTT; maximum efficacy was achieved at doses which maintained near-maximal increases in aPTT (i.e., 2 to 3× increase from baseline) throughout the assessment period (Verhamme, 2021). It was therefore anticipated that a 300 mg dose of REGN9933 would produce maximal efficacy for prevention of VTE in this clinical setting.

### Study Duration and End of Study (EOS)

The duration of the study for a participant was approximately 75 days, excluding the screening period. The definition for end of study (trial) was the date of global last participant's last visit, or the date of withdrawal from the study, or lost to follow-up (i.e., the study participant could no longer be contacted by the investigator).

### Population

The study population comprised adults who were at least 50 years of age and undergoing an elective, unilateral TKA.

### Inclusion Criteria

A participant must have met the following criteria to be eligible for inclusion in the study:
Is a male or female≥50 years of age at the screening visit, undergoing elective unilateral TKA;
Has a body weight≤130 kg at screening visit;
Is judged by the investigator to be in good health based on medical history, physical examination, vital sign measurements, and ECGs performed at screening and/or prior to administration of initial dose of study drug;
Is in good health based on laboratory safety testing obtained during the screening period, including: a. Platelet count>150K; b. Normal PT, international normalized ration (INR), and aPTT during screening; c. Hemoglobin>10 g/dL d. Alanine aminotransferase (ALT) or aspartate aminotransferase (AST)<3× upper level of normal (ULN), or total bilirubin<2× ULN;
Note: Participants with suspected or confirmed Gilbert's disease can be enrolled in the study;
Willing and able to comply with clinic visits and study-related procedures including venography; and
Provide informed consent signed by study participant.

### Exclusion Criteria

A participant who met any of the following criteria was excluded from the study:
Any condition that, as assessed by the investigator, may confound the results of the study or poses an additional risk to the subject by study participation;
History of bleeding in the 6 months prior to dosing requiring hospitalization or transfusion; history of intracranial or intraocular bleeding, excessive operative or postoperative bleeding, and traumatic spinal or epidural anesthesia; history of bleeding diathesis (e.g., Hemophilia A or B, von Willebrand's Factor deficiency);
History of thromboembolic disease or thrombophilia;
History of major surgery, including brain, spinal, or ocular, within approximately the past 6 months;
History of major trauma within approximately the past 6 months prior to dosing;
Hospitalized (>24 hours) for any reason within 30 days of the screening visit;
Using the Modification of Diet in Renal Disease (MDRD) equation, has an estimated glomerular filtration rate (GFR) of <45 mL/min/1.73 m² at the screening visit;
Note: If subject has a GFR below 45 mL/min/1.73 m², one repeat test is allowed. Participant may be enrolled if repeat demonstrates GFR of >45 mL/min/1.73 m².;
History of hypersensitivity or contraindication (per local label) to any of the study treatments or their excipients;
Contraindication to anticoagulation in the opinion of the investigator;
Has received or plans to receive preoperative enoxaparin on the day prior to TKA surgery;
Allergy to contrast agents, poor venous access, or other factors that might preclude venography;
Expected post-operative use of epidural/spinal catheter;
Patients who have traumatic epidural/spinal anesthesia or excessive intra-operative blood loss during TKA surgery should not be randomized to receive study treatment;
Has a history of alcohol or drug abuse;
Known history of human immunodeficiency virus (HIV) infection, or ongoing chronic hepatitis B or hepatitis C infection. Prior resolved hepatitis B infection is not an exclusion;
Any malignancy, except for nonmelanoma skin cancer or cervical/anus in situ, that have been resected with no evidence of metastatic disease for 3 years prior to the screening visit;
History of significant multiple and/or severe allergies (e.g., latex gloves) or has had an anaphylactic reaction to prescription or nonprescription drugs or food;
Participated in any clinical research study evaluating another investigational drug including biologics or therapy, including specific immunotherapy, within 90 days or at least 5 half-lives (whichever is longer) of an investigational biologic drug or at least 4 weeks for other investigational drug prior to the screening visit;
Unwilling or unable to comply with the prohibited medication specifications for this study: a. Participants who require therapeutic (full-dose) anticoagulation should be excluded; b. Participants who require antiplatelet therapy (except for acetylsalicylic acid [ASA]≤100 mg/day) should be excluded;
Presents any concern to the study investigator that might confound the results of the study or poses an additional risk to the subject by their participation in the study;
Members of the clinical site study team and/or his/her immediate family, unless prior approval granted by the sponsor;
Has received a COVID-19 vaccination within 1 week of planned start of study medication or for which the planned COVID-19 vaccinations would not be completed 1 week prior to start of study drug;
Sexually active men with WOCBP partners who are unwilling to use medically acceptable birth control through day 75 (end of study [EOS]): vasectomy with medical assessment of surgical success OR consistent use of a condom;
Women of childbearing potential, defined as defined as women who are fertile following menarche until becoming postmenopausal, unless permanently sterile. Permanent sterilization methods include hysterectomy, bilateral salpingectomy, and bilateral oophorectomy; or
A postmenopausal state is defined as no menses for 12 months without an alternative medical cause. A high follicle-stimulating hormone (FSH) level in the postmenopausal range may be used to confirm a postmenopausal state in women not using hormonal contraception or hormonal replacement therapy. However, in the absence of 12 months of amenorrhea, a single FSH measurement is insufficient to determine the occurrence of a postmenopausal state. The above definitions are according to the Clinical Trial Facilitation Group (CTFG) guidance. Pregnancy testing and contraception are not required for women who are post-menopausal or permanently sterile.

### Treatments

Treatment arms were: REGN9933 300 mg IV, single dose administered as 0.5-hour infusion; Enoxaparin 40 mg SC, daily through the time of venography (or day 12, whichever is earlier); Apixaban 2.5 mg PO, BID through the time of venography (or day 12, whichever is earlier).

For REGN9933, a single IV dose was administered 12 to 24 hours after surgery (>12 hours after removal of the spinal/epidural anesthesia needle/catheter).

For enoxaparin, dosing began 12 to 24 hours after surgery (>12 hours after removal of the spinal/epidural anesthesia needle/catheter). Dosing continued through the time of venography (or day 12, whichever is earlier). If a participant was discharged from the hospital prior to the venogram, the participant could choose to self-administer enoxaparin or have a designated person administer enoxaparin at home. Injection training of enoxaparin was provided. After training, observation of self-administration/designated person was conducted by clinical site personnel. Once this observation was considered satisfactory, then the study drug could be subsequently administered independently by the participant/designated person for the remainder of the dosing period.

For apixaban, dosing began 12 to 24 hours after surgery (>12 hours after removal of the spinal/epidural anesthesia needle/catheter). Dosing continued through the time of venography (or day 12, whichever is earlier). Apixaban dosing continued at home if a participant was discharged from the hospital prior to the venogram.

A patient diary was provided to record compliance of at-home dosing of enoxaparin/apixaban. The diary was completed upon each study drug administration.

TABLE 14
Study Design

| Intervention Name | REGN9933 | Enoxaparin | Apixaban |
|---|---|---|---|
| Intervention | 300 mg IV, single | 40 mg SC, daily | 2.5 mg PO, BID |
| Description | dose administered as | through the time of | through the time of |
| | 0.5-hour infusion | venography | venography |
| Type | Biologic | Drug | Drug |
| Dose Formulation | Lyophilized Powder | Pre-filled Syringe- | Film Coated Tablet |
| | | Liquid Solution | |
| Unit Dose Strength(s) | 265 mg in 20 mL vial | 40 mg, 0.4 mL, pre- | 2.5 mg tablets, in a |
| | | filled syringe | blister pack/wallet |
| | | | carton |
| Dosage Level(s) | 300 mg, once | 40 mg, daily, through | 2.5 mg, BID, through |
| | | the time of | the time of |
| | | venography | venography |
| Route of | IV infusion | SC injection | Oral |
| Administration | | | |

### Endpoints

The primary endpoint is the incidence of confirmed, adjudicated VTE through day 12 (REGN9933 vs enoxaparin). The secondary endpoints are: incidence of major bleeding and clinically relevant nonmajor bleeding through time of venography (or day 12, whichever is earlier); incidence of treatment-emergent adverse events (TEAE) through the end of study; incidence of major VTE through day 12 (REGN9933 vs enoxaparin); incidence of DVT as measured by venography of the operated leg on day 10±2 days (REGN9933 vs enoxaparin); Concentrations of REGN9933 in serum through end of study; change in activated partial thromboplastin time from baseline through end of study; change in prothrombin time from baseline through end of study; incidence and titer of anti-drug antibodies (ADA) to REGN9933 through end of study; and incidence of confirmed, adjudicated VTE through day 12 (enoxaparin vs apixaban). The exploratory endpoints are: Incidence of confirmed VTE through day 12 (REGN9933 vs apixaban) and through the end of study; incidence of major VTE through the end of study; incidence of confirmed symptomatic DVT in either leg through day 12 and through the end of the study; incidence of confirmed PE through day 12 and through the end of study; incidence of fatal PE, which includes sudden death for which PE cannot be ruled out, through day 12 and through the end of study; incidence of DVT as measured by venography of the operated leg on day 10±2 days (REGN9933 vs apixaban); in participants with DVT detected on venogram: distribution of DVT (proximal versus distal), for proximal DVT, size of VTE, for distal DVT, the number of veins affected; incidence of major bleeding and CRNM bleeding through end of study; incidence of minimal bleeding through time of venography and through the end of study; and change in measures of coagulation pathway function compared to baseline, including, but not limited to: FXI:C ([Factor XI functional activity] FXI coagulant activity assay), Total FXI concentration, Thrombin generation assay (TGA) for extrinsic and intrinsic pathways.

### Procedures and Assessments

Efficacy Procedures: Venography of the operated leg was performed on day 10 (+2 days). If DVT or PE was suspected at any point during the study, objective confirmatory testing was carried out.

Safety Procedures: Safety assessments included body weight, height, monitoring of vital signs, physical examination, echocardiogram (ECG), assessment for bleeding, laboratory testing, and the monitoring and reporting of AEs.

Blood samples were collected for drug concentration, total Factor XI concentration, ADA, and exploratory biomarker and research measurements and assessments.

### Statistical Plan

Statistical Hypothesis: The clinical hypothesis comparing the efficacy of REGN9933 to enoxaparin was assessed through a Bayesian estimation framework. Bayesian posterior probabilities were reported to estimate the incidence of confirmed VTE for participants treated with REGN9933 and enoxaparin, as well as the odds ratio (OR) of confirmed VTE in participants administered REGN9933 as compared to that of enoxaparin (OR_{RE}). The statistical hypothesis OR_{RE}<1.

Justification of Sample Size: Up to a total of approximately 360 participants were randomized in a 1:1:1 allocation ratio to REGN9933, apixaban, or enoxaparin. Each arm had an approximate number of 120 participants.

The sample size was determined using the following assumptions: The event rate of VTE for REGN9933 was assumed to be 13% and was set to be 22.4% for enoxaparin; the prior distribution of the treatment difference was assumed to be log(OR)~N(0,0.843), which was expressed as the log(OR). The prior distribution follows a normal distribution centered around no treatment difference and with a variance such that extreme treatment differences are expected to be unlikely, i.e., the probability that OR is between 0.25 and 4 is 90%.

Using these assumptions, and for the purpose of choosing a sample size, the following quantities were simulated: the probability of any beneficial effect of REGN9933 over enoxaparin, i.e., the posterior distribution of the odds ratio comparing REGN9933 to enoxaparin of confirmed VTE. A beneficial effect is where OR_{RE}<1, and thus the study will estimate P(OR_{RE}<1|data); in 75% of the simulated trials, in studies where the P(OR_{RE}<1|data)>0.95, the sample size was 120 participants or less. Therefore, a sample size of 120 subjects per group was chosen.

Statistical Methods: The clinical objectives were estimated using the posterior distribution of OR_{RE}. For comparison of REGN9933 vs enoxaparin, different posterior probabilities will be provided, such as P(OR<0.5, 0.9, 1.0, 1.1, 1.2 etc. data).

The entire posterior density function for the treatment effect (OR_{RE} and the OR of confirmed VTE in participants administered REGN9933 as compared to that of apixaban) was computed, along with the cumulative posterior distribution, which quantifies evidence for all possible treatment effects. A prior for treatment difference log(OR)~N(0,0.843) will be used. Bayesian summary statistics including two-sided 95% Highest Density Interval, posterior mean, and median will be reported. Summary statistics based on the raw data including the number of participants reflected in the calculation (n), mean, standard deviation, Q1, median, Q3, minimum, and maximum were reported.

The Kaplan-Meier plot for cumulative incidence of confirmed VTE over time was provided by treatment groups. Subjects dropped out before the venogram or with missing venograms were considered as censored.

Safety was assessed by clinical and statistical review of all relevant parameters, including vital signs, physical examinations, ECGs, laboratory evaluations, and AEs.

### Results

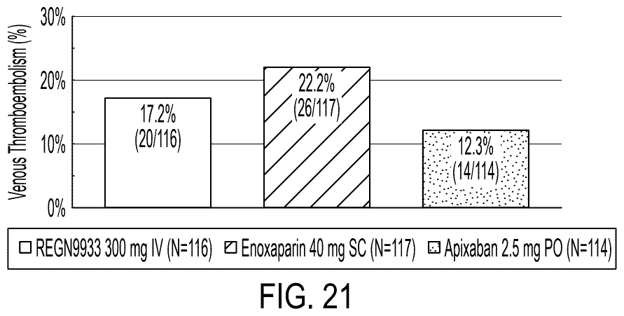

After administration of 300 mg IV REGN9933 12-24 hours post-surgery, the rate of VTE in the study population was 17.2%, see Table 13 and FIG. 21. The previously-published event rate for a placebo group was ~60%.

On the measure of venous thromboembolism (VTE) rates following surgery, a pooled analysis across both trials (Examples 8 and 9) showed REGN7508 was superior to enoxaparin and noninferior to apixaban, and REGN9933 was non-inferior to enoxaparin. All VTE events were asymptomatic, except for one symptomatic case of pulmonary embolism in the apixaban arm. Additionally, no major bleeding (including surgical site bleeding) or clinically relevant non-major bleeding occurred in any study arm. The only treatment-related AE in any arm was one case of minimal bleeding (contusion) reported in the enoxaparin arm. There were no treatment-related serious AEs (SAEs) in any arm.

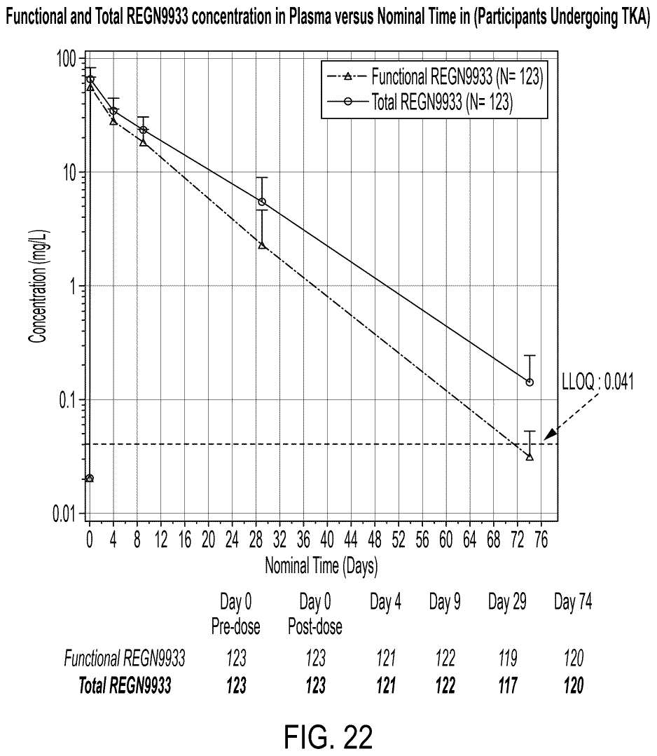

The concentration-time profiles of functional REGN9933 (all mAb regardless of binding status) and total REGN9933 (mAb with 1 or 2 free binding sites) declined in a biphasic manner following IV administration, indicating an initial distribution phase and a subsequent disposition phase (FIG. 22).

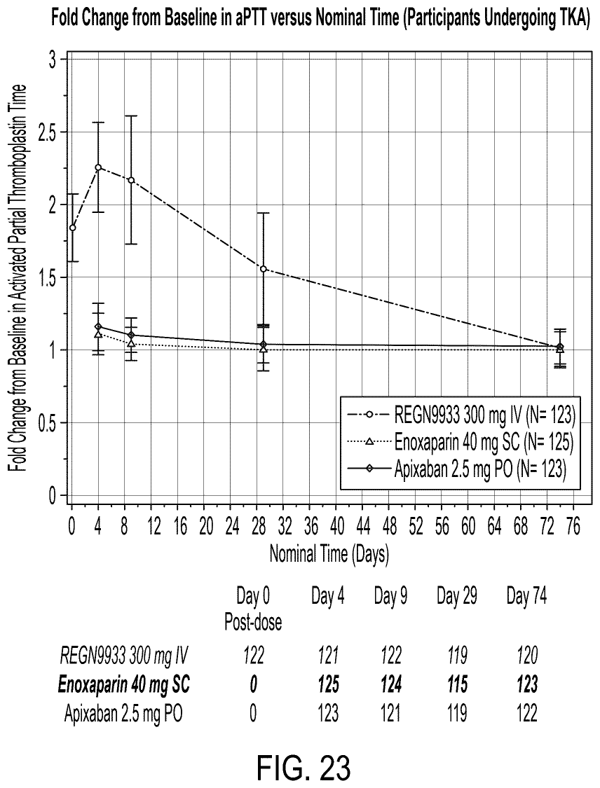

At the end of infusion of REGN9933, aPTT was already ~1.8 fold above baseline. Maximal aPTT prolongation of ~2.3-fold occurred 4 days postdose and values returned to baseline by 74 days postdose. The other treatment arms showed no apparent impact on aPTT (FIG. 23).

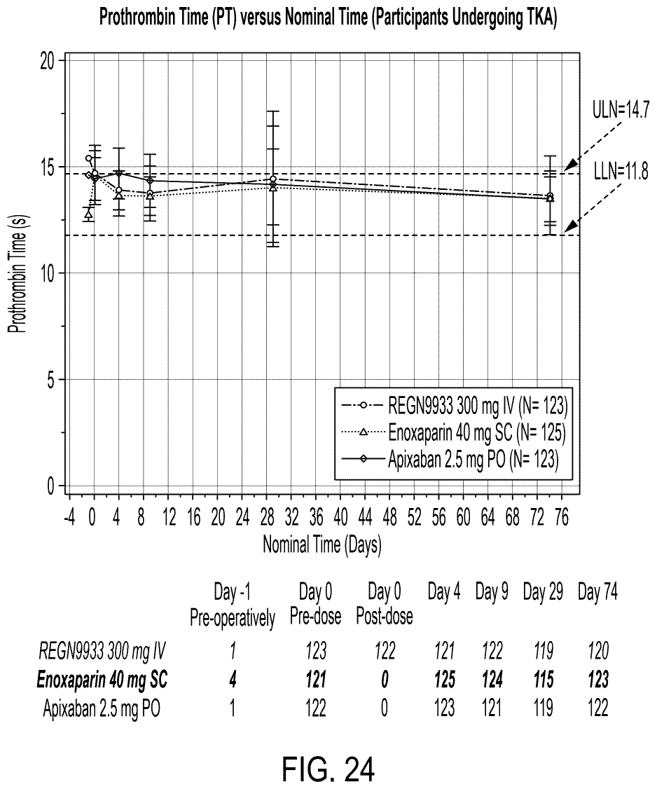

There were no apparent effects on PT following REGN9933, enoxaparin, or apixaban (FIG. 24).

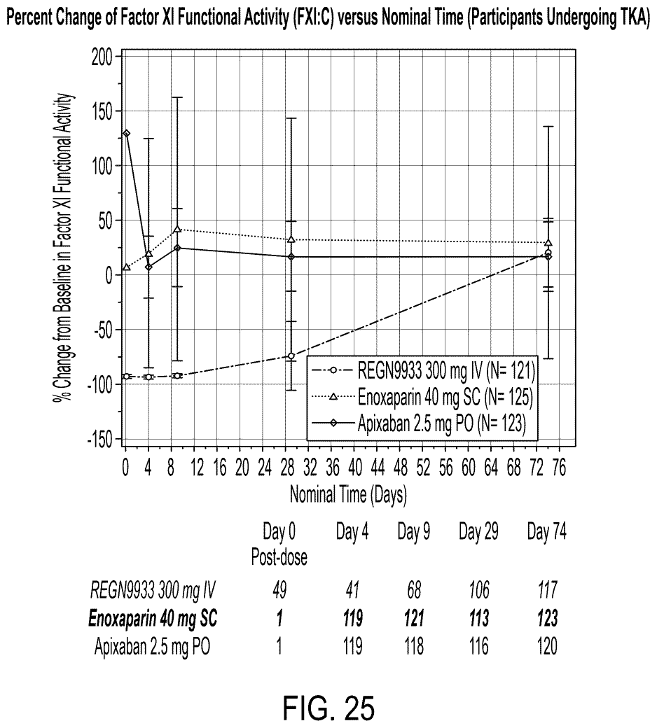

REGN9933 treatment produced a rapid decrease in FXI:C that was sustained through day 9 and returned to baseline by day 74. Maximum effects occurred generally at the first measured timepoint after IV drug administration. There was no observed impact on FXI:C from the other treatment groups (FIG. 25).

### Example 10: A Phase 3, Multicenter, Randomized, Open-Label, Sponsor-Blinded, Study to Evaluate REGN7508 Versus Apixaban and Enoxaparin for Prophylaxis of Venous Thromboembolism (VTE) after Elective Total Knee Arthroplasty (TKA)

This is a phase 3, multicenter, multinational, randomized, open-label, sponsor-blinded, active comparator-controlled study evaluating the efficacy, safety, and tolerability of a single dose of intravenous or subcutaneous REGN7508 administered at two different timepoints compared to standard of care anticoagulants apixaban and enoxaparin for the prophylaxis of VTE in participants undergoing elective TKA.

Excluding the screening period, the study duration is 90 days, which includes a treatment period of 12 days (±2 days) lasting from day of surgery through time of venography, and a follow-up period lasting through the EOS/ET visit on day 90. The screening period may vary from 1-30 days prior to TKA surgery.

Participants will be randomized on day 1 in a 4:1:4:1 ratio to receive either a single dose of REGN7508 250 mg IV within 3 hours of wound closure, a single dose of REGN7508 300 mg SC administered 2 to 4 hours after wound closure, standard of care apixaban 2.5 mg orally (PO) twice daily (BID), or standard of care enoxaparin 40 mg SC once daily (QD).

Participants randomized to SC REGN7508, apixaban, or enoxaparin will also receive an IV placebo infusion within ≤3 hours of wound closure after surgery. Treatment assignment will remain blinded until the end of IV infusion.

Randomization will occur prior to surgery. At the time of wound closure, the investigator must document that the participant continues to meet all inclusion/exclusion criteria and adequate hemostasis has been achieved prior to the blinded first study drug administration (IV REGN7508 or placebo). Participants who do not receive the blinded IV infusion are withdrawn from all further dosing in the study and may receive standard of care treatment at the investigator's discretion.

Treatment after the IV infusion will be administered in an open-label manner because each arm has a unique route of administration, making blinding operationally challenging. In addition, performing the study in a blinded manner would require 90% of participants to receive daily placebo injections, increasing participant burden and discomfort. The investigator will be blinded to individual treatment assignment through completion of the IV infusion. The sponsor will be blinded to individual treatment for the duration of the study. To further mitigate bias, the study will be performed in a blinded-endpoint design, with adjudication of suspected VTE and bleeding (i.e., results of venograms and any confirmatory study for symptomatic VTE; categorization of bleeding events) performed in a blinded fashion by a centralized Independent Adjudication Committee.

### Selected Inclusion Criteria:

Is a male or female≥18 (or the legal age of consent in the jurisdiction in which the study is taking place) years of age at the time of signing the informed consent.
Is undergoing a primary elective unilateral TKA.
Is in good health based on laboratory safety testing:
   Platelet count>100K
   INR/PT and aPTT no more than 25% greater than ULN
   Hemoglobin>10 g/dL
Body weight≤130 kg at screening visit, unless a radiology consultation can assure venography can be adequately performed

### Selected Exclusion Criteria:

Any condition that, as assessed by the investigator, may confound the results of the study or pose an additional risk to the participant by study participation.
History of bleeding in the 6 months prior to randomization requiring hospitalization or transfusion; history of intracranial or intraocular bleeding, excessive operative or post-operative bleeding, and traumatic spinal or epidural anesthesia; history of bleeding diathesis (e.g., Hemophilia A or B, von Willebrand's Factor deficiency).
History of thromboembolic disease or thrombophilia.
History of major surgery, including brain, spinal, or ocular, within approximately the past 6 months.
History of major trauma within approximately the past 6 months prior to randomization.
History of hypersensitivity or contraindication (per local label) to any of the study treatments or their excipients.
Contraindication to anticoagulation in accordance with the enoxaparin and apixaban product labels or in the opinion of the investigator.
Has received or plans to receive preoperative enoxaparin on the day prior to TKA surgery.

### Study Arms and Duration:

### Arm 1: IV REGN7508

REGN7508 250 mg IV, single dose
Dose will be initiated within 3 hours of wound closure at the end of surgery, provided hemostasis has been established

### Arm 2: IV Placebo, Followed by SC REGN7508

REGN7508 300 mg SC, single dose
IV placebo dose will be initiated within 3 hours of wound closure at the end of surgery
SC REGN7508 dose will be initiated 2 to 4 hours* after wound closure at the end of surgery, provided hemostasis has been established
*SC dose cannot be administered until the placebo IV infusion has been completed

### Arm 3: IV Placebo, Followed by Apixaban Active Comparator

Apixaban 2.5 mg PO BID
IV placebo dose will be initiated within 3 hours of wound closure at the end of surgery
Apixaban dosing will be initiated 12 to 24 hours after wound closure at the end of surgery, provided hemostasis has been established, and will continue twice daily through time of venography

### Arm 4: IV Placebo, Followed by Enoxaparin Active Comparator

Enoxaparin 40 mg SC QD
IV placebo dose will be initiated within 3 hours of wound closure at the end of surgery
Enoxaparin dosing will be initiated 12 to 24 hours after wound closure at the end of surgery, provided hemostasis has been established, and will continue once daily through time of venography

The proposed single dose of 250 mg IV of REGN7508 is based primarily on the observed safety, efficacy, and pharmacodynamic data from the phase 2 study in participants undergoing unilateral TKA (R7508-DVT-2360) who received a single 250 mg IV dose of REGN7508 12 to 24 hours post-surgery. REGN7508 markedly reduced the occurrence of asymptomatic lower extremity ipsilateral DVT compared with enoxaparin (7% versus 17%) without any reported major or CRNM bleeding.

The goal of the selected SC dose is to approximately match the same level of maximal aPTT prolongation as that obtained in the phase 2 study, when 250 mg of REGN7508 was administered IV 12 to 24 hours post-surgery. Informed from a model-based exposure-response analysis, the proposed SC dose of 300 mg when administered 2 to 4 hours after surgery is predicted to provide comparable magnitude and onset of PD effect (FXI:C inhibition and aPTT prolongation) relative to the time of surgery as 250 mg IV when the IV dose is administered 12 to 24 hours post-surgery (as was done in the phase 2 study). Additionally, the proposed SC dose of 300 mg is expected to provide maximum inhibition of the FXI coagulation pathway for at least 14 days post-surgery.

### Example 11: Assessing the Impact of REGN9933A2 and REGN7508CAT on Coagulation Relative to Other FXI-Targeted mAbs (Comparator 1 and Comparator 2) and Small Molecules (Small Molecule 1 and 2) In Vitro

To assess the impact of REGN9933 (A2 domain binder) and REGN7508 (catalytic domain binder) on coagulation relative to other FXI-targeted mAbs, which are FXI catalytic domain binder antibodies (comparator ab1 (Koch et al., Blood. 2019; 133(13):1507-1516) and comparator ab2 (Schaefer et al., J Mol Biol. 2019; 431(24):4817-4833)) and small molecules (small molecule 1 (described in U.S. Pat. No. 9,453,018) and small molecule 2 (https://pubchem.ncbi.nlm.nih.gov/compound (PubChem CID 135206011)), human and cynomolgus monkey plasma were used in an in vitro functional clotting assay.

### Methods

aPTT Assay

The activated partial thromboplastin time (aPTT) in plasma was determined using a STA Compact Max Analyzer. Test sample comprised of human plasma with an anti-Factor XI antibody or a small molecule inhibitor of Factor XI. The control sample was human plasma without the antibody or a small molecule inhibitor of Factor XI. Briefly, a total of 50 μl of human plasma sample was added to a cuvette and incubated at 37 °C for 240 sec. 50 ul of aPTTXL (Ellagic acid), was added to the sample as an activator, followed by the addition of 50 ul calcium chloride solution to start the reaction. The measured clot time for the test sample and the control sample were measured; the ratio of the clot time of the test sample relative to the control sample were plotted against concentration of the Factor XI.

### PT Assay

The prothrombin time (PT) in plasma was determined using a STA Compact Max Analyzer. Test sample comprised of human plasma with an anti-Factor XI antibody or a small molecule inhibitor of Factor XI. The control sample was human plasma without the antibody or a small molecule inhibitor of Factor XI. Briefly, a total of 50 μl of plasma sample was added to a cuvette and incubated at 37 °C for 240 sec. The analyzer added 100 ul of STA-NEO CL Plus PT Excel S (Tissue Factor) to start the reaction. and the control sample were measured; the ratio of the clot time of the test sample relative to the control sample were plotted against concentration of the Factor XI.

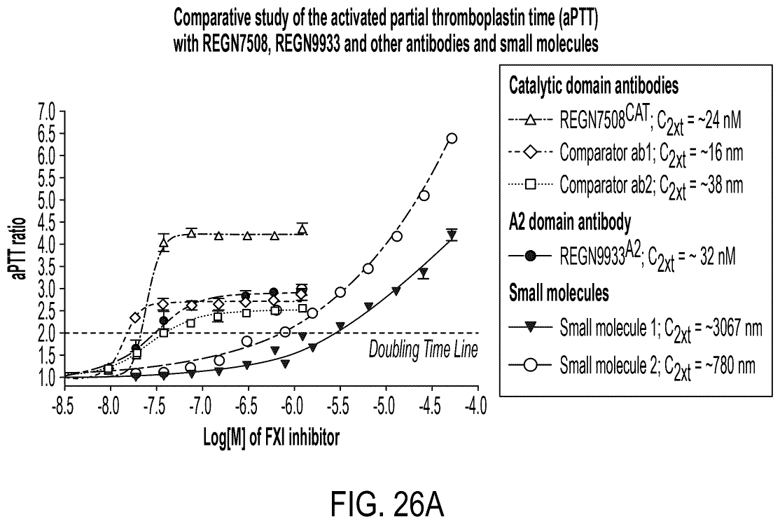

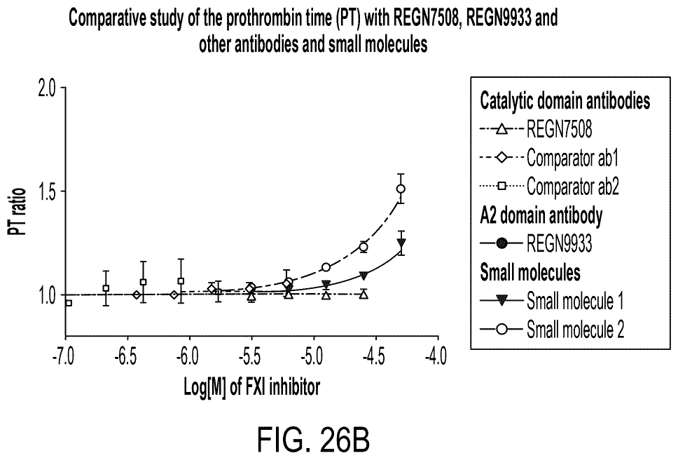

Addition of REGN9933/REGN7508 to plasma resulted in prolonged aPTT (intrinsic-mediated coagulation), while neither mAb showed an effect on PT (extrinsic-mediated coagulation) up to the maximum concentration of 25 μM (FIGS. 26A and 26B). REGN9933 and REGN7508 more potently prolonged aPTT in human plasma compared to monkey plasma, consistent with 1- to 2-fold reduced affinity to monkey FXI. REGN7508 showed greater maximal aPTT prolongation relative to REGN9933. Notably, REGN7508 also showed substantially greater aPTT prolongation than two other FXI-targeted mAbs that are also catalytic-blockers (comparator ab1 and comparator ab2), suggesting that it is a more complete blocker of FXI function. REGN9933, which is a A2 domain binder exhibited comparable aPTT prolongation to comparator ab1 and ab2, which are catalytic domain binders. The small molecules 1 and 2 required much higher concentrations (approximately 10 uM and 3 uM, respectively) to reach a similar aPTT ratio as REGN9933, comparator ab1, and comparator ab2, and much higher concentrations to reach a similar aPTT ratio as REGN7508 (FIG. 26A). At the high concentrations that these small molecules required to approach the aPTT prolongation achieved by the antibodies, the small molecules exhibited “off-target” activity as evidenced by PT extension; none of the mAbs showed effects on PT, indicating higher specificity (FIG. 26B).

### Example 12: Mechanistic Population PK/PD Modeling of Factor XI-Targeting Monoclonal Antibodies REGN7508^{CAT} and REGN9933^{a2} in Healthy Volunteers

### Background

Factor XI (FXI) inhibition is a promising antithrombotic strategy with potentially lower bleeding risk than standard anticoagulants. REGN7508^{CAT} and REGN9933^{A2} are high-affinity, human monoclonal antibodies (mAbs) binding distinct FXI epitopes (catalytic and A2 domains, respectively) to block downstream intrinsic pathway activation. Quantitative pharmacokinetic (PK)/pharmacodynamic (PD) models were developed in healthy volunteers (HVs) to characterize antibody disposition, FXI engagement, and activated partial thromboplastin time (aPTT) response, and to identify clinical subcutaneous (SC) regimens that maximize PD effect throughout dosing for both mAbs.

### Objectives

(1) Build mechanistic target-mediated drug disposition (TMDD) models for total and functional drug and aPTT for REGN7508^{CAT} and REGN9933^{A2} in HVs; (2) quantify covariate effects on PK/PD; (3) run model-based simulations to identify SC regimens expected to maintain maximal PD effect in ≥95% of individuals under variable physiologic and pathologic conditions.

### Methods

Data were integrated from two first-in-human, single-ascending-dose studies in HVs (6:2 active:placebo). REGN7508^{CAT} was administered intravenously (IV, 5-250 mg; six cohorts) or SC (125-600 mg; four cohorts; NCT05603195); REGN9933^{A2} was administered IV (3-300 mg; five cohorts) or SC (100 and 300 mg; NCT05102136). PK/PD modeling was conducted in MONOLIX® 2024R1 using a bivalent TMDD framework accounting for free antibody, single-arm-bound functional complex, and fully-bound complex. Functional drug (free+single-arm-bound antibody) directly drove aPTT via a direct-response model. Allometric body-weight scaling was applied to PK; covariates were screened with the Conditional Sampling for Stepwise Approach based on Correlation (COSSAC) procedure. Simulations were performed in SIMULX® to compare biweekly (Q2W) and monthly (Q4W) SC regimens for REGN7508^{CAT} and evaluate Q2W for REGN9933^{A2} under baseline settings and stress-test conditions (increased antibody clearance and reduced bioavailability), representing diverse patient populations and disease states.

### Results

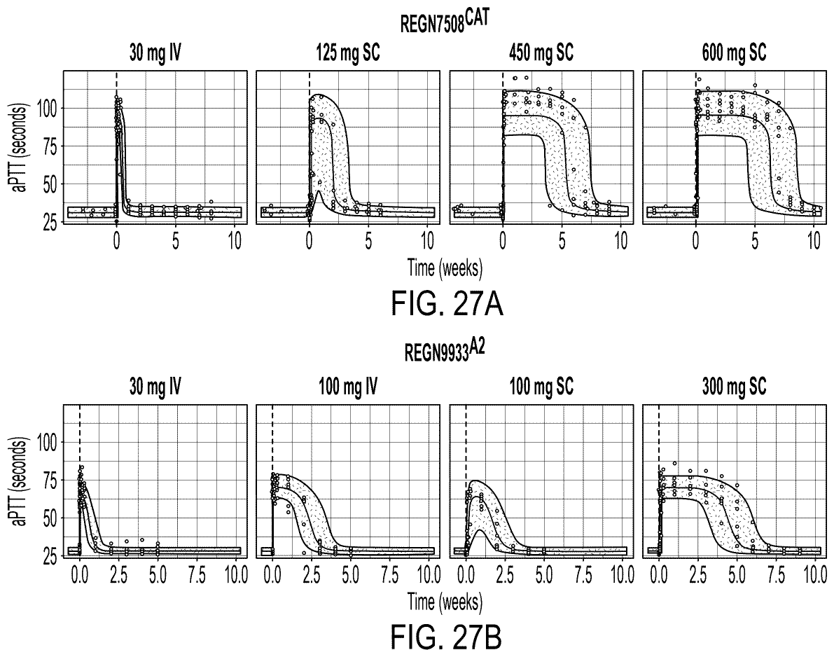

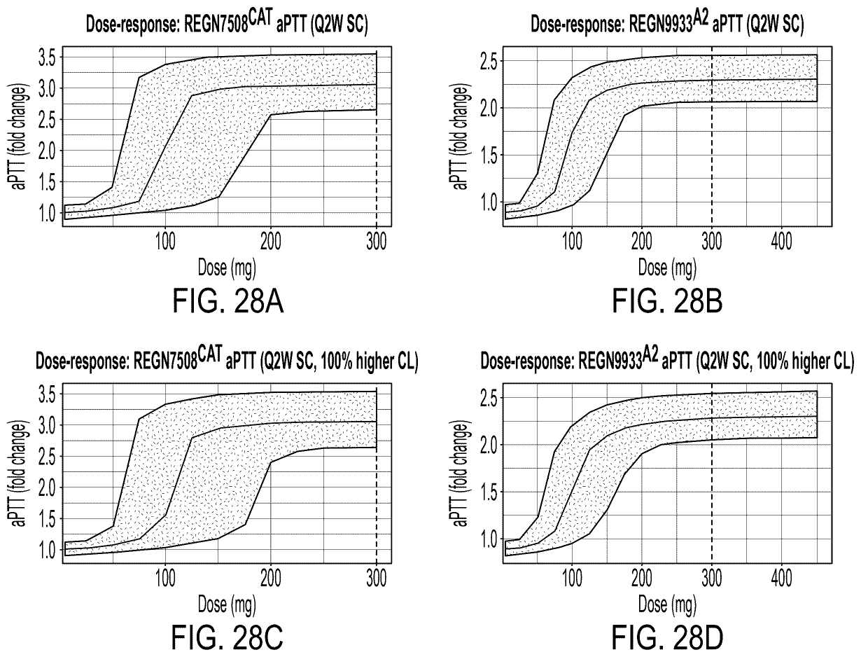

Both TMDD models adequately captured nonlinear PK and aPTT dynamics following IV and SC dosing (FIGS. 27A-27B). REGN7508^{CAT} and REGN9933^{A2} achieved dose-dependent aPTT prolongation (at sustained PD plateaus, with medians of ~3.2× for REGN7508^{CAT} and ~2.5× for REGN9933^{A2} relative to baseline aPTT), consistent with target saturation and epitope binding. Model-predicted 90% intervals indicated that ≥95% of individuals would achieve maximal or near-maximal aPTT prolongation at ≥250-300 mg SC Q2W. Simulated dose-response relationships showed a durable PD effect and minimal rightward shift when clearance doubled, supporting regimen robustness under stress (FIGS. 28A-28D). Estimated antibody half-lives were ~23 days (REGN7508^{CAT}) and ~26 days (REGN9933^{A2}); free FXI half-life was ~50-80 hours. Age emerged as a positive covariate on EC₅₀ for REGN7508^{CAT}, suggesting reduced sensitivity to catalytic domain inhibition with increasing age, with negligible impact on predicted dose response. Both models predict a plateau in aPTT response beyond 250-300 mg, indicating near-maximal FXI suppression, with only modest rightward shifts in dose-response curves under increased clearance conditions.

### Conclusions

Mechanistic TMDD modeling for REGN7508^{CAT} and REGN9933^{A2} quantitatively linked exposure to aPTT response in HVs and supported 300 mg SC Q2W as the more robust regimen to maintain maximal PD effect in ≥95% of individuals. Robustness persisted under simulated increases in clearance and decreases in bioavailability. These HV models provide a mechanistic basis for dose selection across multiple indications, and translation to patient populations with variable physiology or disease-related covariates, including patients in post-operative venous thromboembolism and other future Phase 2/3 programs.

1. A method of preventing, managing, or reducing the risk of a venous thromboembolism (VTE), or a complication or symptom associated with VTE following a surgical procedure in a subject, the method comprising:
administering an antibody, or antigen-binding portion thereof, that binds to Factor XI (FXI) to the subject,
wherein the antibody, or antigen-binding portion thereof, is administered at a dose of about 200 mg to about 600 mg,
wherein the antibody, or antigen-binding portion thereof, comprises a heavy chain complementarity determining region (HCDR) 1, HCDR2, and HCDR3 within a heavy chain variable region (HCVR) comprising the amino acid sequence of SEQ ID NO: 23; and a light chain complementarity determining region (LCDR) 1, LCDR2, and LCDR3 within a light chain variable region (LCVR) comprising the amino acid sequence of SEQ ID NO: 31;
thereby preventing, managing, or reducing the risk of VTE, or the complication or symptom associated with VTE, following the surgical procedure in the subject.

2. A method of preventing, managing, or reducing the risk of a venous thromboembolism (VTE), or a complication or symptom associated with VTE, following a surgical procedure in a subject, the method comprising:
administering an antibody, or antigen-binding portion thereof, that binds to FXI to the subject,
wherein the antibody, or antigen-binding portion thereof, is administered at a dose of about 200 mg to about 600 mg,
wherein the antibody, or antigen-binding portion thereof, comprises a heavy chain complementarity determining region (HCDR) 1, HCDR2, and HCDR3 within a heavy chain variable region (HCVR) comprising the amino acid sequence of SEQ ID NO: 3; and a light chain complementarity determining region (LCDR) 1, LCDR2, and LCDR3 within a light chain variable region (LCVR) comprising the amino acid sequence of SEQ ID NO: 13;
thereby preventing, managing, or reducing the risk of the VTE, or the complication or symptom associated with VTE, following the surgical procedure in the subject.

3. The method of claim 1, wherein the antibody, or antigen-binding portion thereof, is administered as an intravenous dose of about 250 mg.

4. The method of claim 3, wherein the antibody, or antigen-binding portion thereof, is administered as a single intravenous dose of 250 mg within 3 hours of would closure after surgery.

5. The method of claim 1, wherein the antibody, or antigen-binding portion thereof, is administered as a subcutaneous dose of about 250 mg to about 600 mg.

6. The method of claim 5, wherein the antibody, or antigen-binding portion thereof, is administered as a single subcutaneous dose of 300 mg within 2 to 4 hours after surgery.

7. The method of claim 2, wherein the antibody, or antigen-binding portion thereof, is administered as a single intravenous dose of about 300 mg.

8. The method of claim 2, wherein the antibody, or antigen-binding portion thereof, is administered as a single subcutaneous dose of about 300 mg to about 450 mg.

9.-11. (canceled)

12. The method of claim 7, wherein the administering occurs between 2 hours and 24 hours after the surgical procedure.

13. The method of claim 12, wherein the administering occurs between 12 hours and 24 hours after the surgical procedure.

14. The method of claim 1, wherein the HCDR1 comprises SEQ ID NO: 25, the HCDR2 comprises SEQ ID NO: 27, and the HCDR3 comprises SEQ ID NO: 29, and wherein the LCDR1 comprises SEQ ID NO: 33, the LCDR2 comprises SEQ ID NO: 35, and the LCDR3 comprises SEQ ID NO: 37.

15. (canceled)

16. The method of claim 1, wherein the antibody, or antigen-binding portion thereof, that binds to FXI comprises a heavy chain (HC) comprising SEQ ID NO: 21, and a light chain (LC) comprising SEQ ID NO: 39.

17. The method of claim 2, wherein the HCDR1 comprises SEQ ID NO: 5, the HCDR2 comprises SEQ ID NO: 7, and the HCDR3 comprises SEQ ID NO: 9, and wherein the LCDR1 comprises SEQ ID NO: 15, the LCDR2 comprises SEQ ID NO: 17, and the LCDR3 comprises SEQ ID NO: 19.

18. (canceled)

19. The method of claim 2, wherein the antibody, or antigen-binding portion thereof, that binds to FXI comprises a heavy chain (HC) comprising SEQ ID NO: 1, and a light chain (LC) comprising SEQ ID NO: 11.

20.-25. (canceled)

26. The method of claim 1, further comprising administering one or more anti-coagulants in combination with the antibody, or antigen-binding portion thereof, wherein the one or more anti-coagulants is selected from the group consisting of: aspirin, enoxaparin, apixaban and rivaroxaban.

27. (canceled)

28. The method of claim 27, wherein the anti-coagulant is aspirin, and wherein the aspirin is administered at a dosage of about 50 mg to about 100 mg per day, or about 75 mg per day, for at least 5 days or at least 12 days following the surgical procedure.

29. (canceled)

30. The method of claim 1, wherein the surgical procedure is a cardiac, vascular, thoracic, orthopedic, trauma, abdominopelvic, gynecologic, urologic, bariatric, plastic, reconstructive, otolaryngic, arthroscopic, organ transplant, or neurologic surgery.

31. The method of claim 1, wherein the surgical procedure is selected from the group consisting of total knee arthroplasty (TKA), hip arthroplasty, spinal cord injury surgery, and microvascular decompression surgery.

32. The method of claim 1, wherein the complication or symptom associated with VTE is lower extremity swelling, warmth, redness, pain, shortness of breath, chest pain exacerbated by inspiration, hemoptysis, bleeding at the surgical site or elsewhere, or a combination thereof.

33. The method of claim 1, wherein the preventing, managing, or reducing the risk of a VTE, or a complication or symptom associated with VTE following a surgical procedure is achieved without substantially increasing the bleeding risk of the subject, and/or wherein administration of the antibody, or antigen-binding portion thereof, to the subject does not substantially increase the bleeding risk in the subject as compared to subjects undergoing the surgical procedure who have not been administered the antibody, or antigen-binding portion thereof.

34. The method of claim 1, wherein the risk of thrombosis in the subject is decreased by at least 10%, at least 25%, at least 35%, at least 50%, at least 75%, at least 80%, at least 85%, at least 90%, or at least 95% following the administering step as compared to a control, wherein the control is an average measurement of thrombosis gathered from a population of subjects undergoing the surgical procedure who have not been administered the antibody, or antigen-binding portion thereof.

35. (canceled)

36. The method of claim 1, wherein the administering results in prolonging activated partial thromboplastin time (aPTT), or reducing thrombin activity in plasma of the subject.

37. The method of claim 1, wherein the antibody, or antigen-binding fragment thereof,
(a) increases activated partial thromboplastin time (aPTT) by at least two-fold, by at least 2.5-fold, by at least 3-fold, by at least 3.5-fold, or by at least 3.8-fold,
(b) does not increase prothrombin time (PT),
(c) inhibits FXIa-mediated thrombin activity by at least 5%, by at least 10%, by at least 15%, or by 5%-15%, and/or
(d) inhibits Factor XI functional activity (FXI:C) by at least 85%, or by at least 90%, and wherein the inhibition is maintained for at least 20, at least 30, or at least 40 days post-dose.

38.-41. (canceled)

42. The method of claim 1, wherein the antibody, or antigen binding portion thereof, is administered intravenously at a dose of about 250 mg to achieve:
(a) a rate of clearance (CL) of about 0.5 L/day to about 1.0 L/day,
(b) a maximum serum concentration (Cₘₐₓ) of about 42.0 mg/L to about 47.0 mg/L,
(c) a dose-normalized serum concentration (C_{max/dose}) of about 0.10 mg/L/mg to 0.2 mg/L/mg,
(d) a steady-state volume of distribution (Vₛₛ) of about 5.0 L to about 7.5 L,
(e) a time to Cₘₐₓ (Tₘₐₓ) of about 0.5 days to about 1.5 days,
(f) a time to last measurable serum concentration (Tₗₐₛₜ) of about 27 days to about 32 days,
(g) a serum concentration half-life (T_{1/2}) of about 4.5 days to about 5.5 days,
(h) an area under the plasma concentration-time curve to last measurable serum concentration (AUCₗₐₛₜ) of about 370 day*mg/L to about 380 day*mg/L,
(i) a dose-normalized area under the plasma concentration-time curve from administration to last measurable serum concentration (AUC_{last/dose}) of about 1.4 day*mg/L/mg to about 1.6 day*mg/L/mg,
(j) an area under the plasma concentration-time curve extrapolated from administration to infinity (AUC_{inf}) of about 385 day*mg/L to about 395 day*mg/L,
(k) a dose-normalized area under the plasma concentration-time curve extrapolated from administration to infinity (AUC_{inf/dose}) of about 1.3 day*mg/L/mg to about 1.7 day*mg/L/mg, and/or
(l) an area under the plasma concentration-time curve extrapolated from administration to infinity as a percentage of the total area under the plasma concentration-time curve (AUCₑₓₜᵣₐₚ) of about 1.5% to about 2.5%.

43. The method of claim 2, wherein the antibody, or antigen binding portion thereof, is administered intravenously at a dose of about 300 mg to achieve:
(a) a rate of clearance (CL) of 0.2 to about 0.28 L/day, or about 0.3 L/day to about 0.41 L/day,
(b) a maximum serum concentration (Cₘₐₓ) of about 85 mg/L to about 112 mg/L, or about 45 mg/L to about 65 mg/L,
(c) a steady-state volume of distribution (Vₛₛ) of about 3.0 L to about 4.1 L, or about 4.5 L to about 7 L,
(d) an area under the plasma concentration-time curve to last measurable serum concentration (AUCₗₐₛₜ) of about 1055 day*mg/L to about 1455 day*mg/L, or about 640 day*mg/L to about 910 day*mg/L, and/or
(e) an area under the plasma concentration-time curve extrapolated from administration to infinity (AUC_{inf}) of about 1000 day*mg/L/mg to about 1475 day*mg/L/mg, or about 640 day*mg/L to about 910 day*mg/L.

44. A method of preventing, managing, or reducing the risk of a venous thromboembolism (VTE), or a complication or symptom associated with VTE following a surgical procedure in a subject, the method comprising:
administering an antibody, or antigen-binding portion thereof, that binds to Factor XI (FXI) to the subject,
wherein the antibody, or antigen-binding portion thereof, comprises a heavy chain complementarity determining region (HCDR) 1, HCDR2, and HCDR3 within a heavy chain variable region (HCVR) comprising the amino acid sequence of SEQ ID NO: 23; and a light chain complementarity determining region (LCDR) 1, LCDR2, and LCDR3 within a light chain variable region (LCVR) comprising the amino acid sequence of SEQ ID NO: 31,
wherein the antibody, or antigen-binding portion thereof, is administered as a single intravenous dose of about 250 mg,
wherein the administering occurs within 3 hours of wound closure after the surgical procedure,
thereby preventing, managing, or reducing the risk of VTE, or the complication or symptom associated with VTE, following the surgical procedure in the subject.

45. The method of claim 44, wherein the antibody, or the antigen-binding portion thereof, that binds to FXI comprises a heavy chain (HC) comprising SEQ ID NO: 21 and a light chain (LC) comprising SEQ ID NO: 39.

46. (canceled)

47. A method of preventing, managing, or reducing the risk of a venous thromboembolism (VTE), or a complication or symptom associated with VTE, following a surgical procedure in a subject, the method comprising:
administering an antibody, or antigen-binding portion thereof, that binds to FXI to the subject,
wherein the antibody, or antigen-binding portion thereof, comprises a heavy chain complementarity determining region (HCDR) 1, HCDR2, and HCDR3 within a heavy chain variable region (HCVR) comprising the amino acid sequence of SEQ ID NO: 3; and a light chain complementarity determining region (LCDR) 1, LCDR2, and LCDR3 within a light chain variable region (LCVR) comprising the amino acid sequence of SEQ ID NO: 13,
wherein the antibody, or antigen-binding portion thereof, is administered as a single intravenous dose of about 300 mg,
wherein the administering occurs within 24 hours after the surgical procedure,
thereby preventing, managing, or reducing the risk of the VTE, or the complication or symptom associated with VTE, following the surgical procedure in the subject.

48. The method of claim 47, wherein the antibody, or the antigen-binding portion thereof, that binds to FXI comprises a heavy chain (HC comprising SEQ ID NO: 1 and a light chain (LC) comprising SEQ ID NO: 11.

49.-51. (canceled)

52. The method of claim 5, wherein the antibody, or antigen-binding portion thereof, is administered as a single subcutaneous dose of about 250 mg to about 600 mg before surgery.

53. The method of claim 8, wherein the antibody, or antigen-binding portion thereof, is administered as a single subcutaneous dose of about 300 mg to about 450 mg before surgery.

54. The method of claim 11, wherein the administering occurs within 3 hours of wound closure after surgery.

55. The method of claim 47, wherein the administering occurs within 3 hours of wound closure after surgery.


SEQUENCE LISTING (48 sequences)

SEQ ID NO: 1 - protein, 450 aa, synthetic construct
  REGION 1..450: note = Synthetic
EVQLVESGGGLVQPGGSLRLSCAASGFTFSNYAMNWVRQAPGKGLDWVSFIRSGGDTTYYADSVKGRFTISRDNSKNTLYLQMNSLRAEDTAVYYCAKVHPYTWDYGDAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - DNA, 1353 bp, synthetic construct
  misc_feature 1..1353: note = Synthetic
  source 1..1353: mol_type = other DNA
gaggtgcagctggtggagtctgggggaggcttggtacagcctggggggtccctgagactctcctgtgcagcctctggattcacctttagcaactatgccatgaactgggtccgccaggctccagggaaggggctggactgggtctcatttattcgtagtggtggtgataccacatactatgcagactccgtgaagggccggttcaccatctccagagacaattccaagaacacgctgtatctgcagatgaacagcctgagagccgaggacacggccgtatattactgtgcgaaggttcatccatatacctgggattacggtgatgcttttgatatctggggccaggggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 3 - protein, 123 aa, synthetic construct
  REGION 1..123: note = Synthetic
EVQLVESGGGLVQPGGSLRLSCAASGFTFSNYAMNWVRQAPGKGLDWVSFIRSGGDTTYYADSVKGRFTISRDNSKNTLYLQMNSLRAEDTAVYYCAKVHPYTWDYGDAFDIWGQGTMVTVSS

SEQ ID NO: 4 - DNA, 369 bp, synthetic construct
  misc_feature 1..369: note = Synthetic
  source 1..369: mol_type = other DNA
gaggtgcagctggtggagtctgggggaggcttggtacagcctggggggtccctgagactctcctgtgcagcctctggattcacctttagcaactatgccatgaactgggtccgccaggctccagggaaggggctggactgggtctcatttattcgtagtggtggtgataccacatactatgcagactccgtgaagggccggttcaccatctccagagacaattccaagaacacgctgtatctgcagatgaacagcctgagagccgaggacacggccgtatattactgtgcgaaggttcatccatatacctgggattacggtgatgcttttgatatctggggccaggggacaatggtcaccgtctcttca

SEQ ID NO: 5 - protein, 8 aa, synthetic construct
  REGION 1..8: note = Synthetic
GFTFSNYA

SEQ ID NO: 6 - DNA, 24 bp, synthetic construct
  misc_feature 1..24: note = Synthetic
  source 1..24: mol_type = other DNA
ggattcacctttagcaactatgcc

SEQ ID NO: 7 - protein, 8 aa, synthetic construct
  REGION 1..8: note = Synthetic
IRSGGDTT

SEQ ID NO: 8 - DNA, 24 bp, synthetic construct
  misc_feature 1..24: note = Synthetic
  source 1..24: mol_type = other DNA
attcgtagtggtggtgataccaca

SEQ ID NO: 9 - protein, 16 aa, synthetic construct
  REGION 1..16: note = Synthetic
AKVHPYTWDYGDAFDI

SEQ ID NO: 10 - DNA, 48 bp, synthetic construct
  misc_feature 1..48: note = Synthetic
  source 1..48: mol_type = other DNA
gcgaaggttcatccatatacctgggattacggtgatgcttttgatatc

SEQ ID NO: 11 - protein, 215 aa, synthetic construct
  REGION 1..215: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - DNA, 648 bp, synthetic construct
  misc_feature 1..648: note = Synthetic
  source 1..648: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 13 - protein, 108 aa, synthetic construct
  REGION 1..108: note = Synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 14 - DNA, 324 bp, synthetic construct
  misc_feature 1..324: note = Synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 15 - protein, 6 aa, synthetic construct
  REGION 1..6: note = Synthetic
QSISSY

SEQ ID NO: 16 - DNA, 18 bp, synthetic construct
  misc_feature 1..18: note = Synthetic
  source 1..18: mol_type = other DNA
cagagcattagcagctat

SEQ ID NO: 17-18 - skipped (2 entries)

SEQ ID NO: 19 - protein, 10 aa, synthetic construct
  REGION 1..10: note = Synthetic
QQSYSTPPIT

SEQ ID NO: 20 - DNA, 30 bp, synthetic construct
  misc_feature 1..30: note = Synthetic
  source 1..30: mol_type = other DNA
caacagagttacagtacccctccgatcacc

SEQ ID NO: 21 - protein, 445 aa, synthetic construct
  REGION 1..445: note = Synthetic
QVQLVESGGGLVKPGGSLRLSCAASGFTFSDYSMNWIRQAPGKGLEWISYISFSGNSIYYADSVKGRFTISRDNAKNSLYLQMNSLRVEDTAIYYCTSRDWGYAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - DNA, 1338 bp, synthetic construct
  misc_feature 1..1338: note = Synthetic
  source 1..1338: mol_type = other DNA
caggtgcagctggtggagtctgggggaggcttggtcaagcctggagggtccctgagactctcctgtgcagcctctggattcaccttcagtgactactccatgaactggatccgccaggctccagggaaggggctggagtggatctcatacattagttttagtggtaatagcatatactacgcagactctgtgaagggccgattcaccatctccagggacaacgccaagaactccctatatctgcaaatgaacagcctgagagtcgaggacacggccatatattactgtacgagccgggactggggatatgcttttgatatctggggccaagggacaatggtcaccgtctcttcagcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaatga

SEQ ID NO: 23 - protein, 118 aa, synthetic construct
  REGION 1..118: note = Synthetic
QVQLVESGGGLVKPGGSLRLSCAASGFTFSDYSMNWIRQAPGKGLEWISYISFSGNSIYYADSVKGRFTISRDNAKNSLYLQMNSLRVEDTAIYYCTSRDWGYAFDIWGQGTMVTVSS

SEQ ID NO: 24 - DNA, 354 bp, synthetic construct
  misc_feature 1..354: note = Synthetic
  source 1..354: mol_type = other DNA
caggtgcagctggtggagtctgggggaggcttggtcaagcctggagggtccctgagactctcctgtgcagcctctggattcaccttcagtgactactccatgaactggatccgccaggctccagggaaggggctggagtggatctcatacattagttttagtggtaatagcatatactacgcagactctgtgaagggccgattcaccatctccagggacaacgccaagaactccctatatctgcaaatgaacagcctgagagtcgaggacacggccatatattactgtacgagccgggactggggatatgcttttgatatctggggccaagggacaatggtcaccgtctcttca

SEQ ID NO: 25 - protein, 8 aa, synthetic construct
  REGION 1..8: note = Synthetic
GFTFSDYS

SEQ ID NO: 26 - DNA, 24 bp, synthetic construct
  misc_feature 1..24: note = Synthetic
  source 1..24: mol_type = other DNA
ggattcaccttcagtgactactcc

SEQ ID NO: 27 - protein, 8 aa, synthetic construct
  REGION 1..8: note = Synthetic
ISFSGNSI

SEQ ID NO: 28 - DNA, 24 bp, synthetic construct
  misc_feature 1..24: note = Synthetic
  source 1..24: mol_type = other DNA
attagttttagtggtaatagcata

SEQ ID NO: 29 - protein, 11 aa, synthetic construct
  REGION 1..11: note = Synthetic
TSRDWGYAFDI

SEQ ID NO: 30 - DNA, 33 bp, synthetic construct
  misc_feature 1..33: note = Synthetic
  source 1..33: mol_type = other DNA
acgagccgggactggggatatgcttttgatatc

SEQ ID NO: 31 - protein, 107 aa, synthetic construct
  REGION 1..107: note = Synthetic
DIQMTQSPSFLSASLGDRVTITCQASQDISNYLNWYQVKPGKAPKLLIYDASNLETGVPSRFSGSGSGTDFTFTISSLQPEDIATYFCQQYDNLPYIFGQGTKLEIK

SEQ ID NO: 32 - DNA, 321 bp, synthetic construct
  misc_feature 1..321: note = Synthetic
  source 1..321: mol_type = other DNA
gacatccagatgacccagtctccatccttcctgtctgcatctttaggagacagagtcaccatcacttgccaggcgagtcaggacattagcaattatttaaattggtatcaggtgaagccagggaaagcccctaagctcctgatctacgatgcatccaatttggaaacaggggtcccatcaaggttcagtggaagtggctctgggacagattttactttcaccatcagcagcctgcagcctgaagatattgcaacatatttctgtcaacaatatgataatctcccgtacatttttggccaggggaccaagctggagatcaaa

SEQ ID NO: 33 - protein, 6 aa, synthetic construct
  REGION 1..6: note = Synthetic
QDISNY

SEQ ID NO: 34 - DNA, 18 bp, synthetic construct
  misc_feature 1..18: note = Synthetic
  source 1..18: mol_type = other DNA
caggacattagcaattat

SEQ ID NO: 35-36 - skipped (2 entries)

SEQ ID NO: 37 - protein, 9 aa, synthetic construct
  REGION 1..9: note = Synthetic
QQYDNLPYI

SEQ ID NO: 38 - DNA, 27 bp, synthetic construct
  misc_feature 1..27: note = Synthetic
  source 1..27: mol_type = other DNA
caacaatatgataatctcccgtacatt

SEQ ID NO: 39 - protein, 214 aa, synthetic construct
  REGION 1..214: note = Synthetic
DIQMTQSPSFLSASLGDRVTITCQASQDISNYLNWYQVKPGKAPKLLIYDASNLETGVPSRFSGSGSGTDFTFTISSLQPEDIATYFCQQYDNLPYIFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 645 bp, synthetic construct
  misc_feature 1..645: note = Synthetic
  source 1..645: mol_type = other DNA
gacatccagatgacccagtctccatccttcctgtctgcatctttaggagacagagtcaccatcacttgccaggcgagtcaggacattagcaattatttaaattggtatcaggtgaagccagggaaagcccctaagctcctgatctacgatgcatccaatttggaaacaggggtcccatcaaggttcagtggaagtggctctgggacagattttactttcaccatcagcagcctgcagcctgaagatattgcaacatatttctgtcaacaatatgataatctcccgtacatttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 41 - protein, 625 aa, Homo sapiens
MIFLYQVVHFILFTSVSGECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAV

SEQ ID NO: 42 - protein, 635 aa, synthetic construct
  REGION 1..635: note = Synthetic
ECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAVEQKLISEEDLGGEQKLISEEDLHHHHHH

SEQ ID NO: 43 - protein, 634 aa, synthetic construct
  REGION 1..634: note = Synthetic
GCLTQLYENAFFRGGDVASMYTPNAQYCQMRCTFHPRCLLFSFLPASSINDMEKRFGCFLKDSVTGTLPKVHRTGAVSGHSLKQCHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAVEQKLISEEDLGGEQKLISEEDLHHHHHH

SEQ ID NO: 44 - protein, 633 aa, synthetic construct
  REGION 1..633: note = Synthetic
ECVTQLLKDTCFEGGDITTVFTPSAKYCQVVCTYHPRCLLFTFTAESPSEDPTRWFTCVLKDSVTETLPRVNRTAAISGYSFKQCSHQISCHRDIYKGVDMRGVNFNVSKVSSVEECQKRCTNNIRCQFFSYATQTFHKAEYRNNCLLKYSPGGTPTAIKVLSNVESGFSLKPCLSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAVEQKLISEEDLGGEQKLISEEDLHHHHHH

SEQ ID NO: 45 - protein, 633 aa, synthetic construct
  REGION 1..633: note = Synthetic
ECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLCHMNIFQHLAFSDVDVARVLTPDAFVCRTICTYHPNCLFFTFYTNVWKIESQRNVCLLKTSESGTPSSSTPQENTISGYSLLTCHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAVEQKLISEEDLGGEQKLISEEDLHHHHHH

SEQ ID NO: 46 - protein, 633 aa, synthetic construct
  REGION 1..633: note = Synthetic
ECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVCHSKIYPGVDFGGEELNVTFVKGVNVCQETCTKMIRCQFFTYSLLPEDCKEEKGKCFLRLSMDGSPTRIAYGTQGSSGYSLRLCMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAVEQKLISEEDLGGEQKLISEEDLHHHHHH

SEQ ID NO: 47 - protein, 647 aa, synthetic construct
  REGION 1..647: note = Synthetic
GCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPAEQKLISEEDLGGEQKLISEEDLHHHHHH

SEQ ID NO: 48 - protein, 638 aa, Homo sapiens
MILFKQATYFISLFATVSCGCLTQLYENAFFRGGDVASMYTPNAQYCQMRCTFHPRCLLFSFLPASSINDMEKRFGCFLKDSVTGTLPKVHRTGAVSGHSLKQCGHQISACHRDIYKGVDMRGVNFNVSKVSSVEECQKRCTNNIRCQFFSYATQTFHKAEYRNNCLLKYSPGGTPTAIKVLSNVESGFSLKPCALSEIGCHMNIFQHLAFSDVDVARVLTPDAFVCRTICTYHPNCLFFTFYTNVWKIESQRNVCLLKTSESGTPSSSTPQENTISGYSLLTCKRTLPEPCHSKIYPGVDFGGEELNVTFVKGVNVCQETCTKMIRCQFFTYSLLPEDCKEEKCKCFLRLSMDGSPTRIAYGTQGSSGYSLRLCNTGDNSVCTTKTSTRIVGGTNSSWGEWPWQVSLQVKLTAQRHLCGGSLIGHQWVLTAAHCFDGLPLQDVWRIYSGILNLSDITKDTPFSQIKEIIIHQNYKVSEGNHDIALIKLQAPLNYTEFQKPICLPSKGDTSTIYTNCWVTGWGFSKEKGEIQNILQKVNIPLVTNEECQKRYQDYKITQRMVCAGYKEGGKDACKGDSGGPLVCKHNGMWRLVGITSWGEGCARREQPGVYTKVAEYMDWILEKTQSSDGKAQMQSPA